(19) United States
(12) Patent Application Publication
(10) Pub. No.: US 2023/0285572 A1
(43) Pub. Date: Sep. 14, 2023

(54) TARGETED DELIVERY OF THERAPEUTIC AGENTS

(71) Applicant: Sepelo Therapeutics, LLC, San Marino, CA (US)

(72) Inventors: Emil SAMARA, San Marino, CA (US); Tomasz GLINKA, Cupertino, CA (US); Wolf-Nicolas FISCHER, Sunnyvale, CA (US); Bernd JANDELEIT, Menlo Park, CA (US)

(21) Appl. No.: 18/019,776

(22) PCT Filed: Aug. 3, 2021
(86) PCT No.: PCT/US2021/044417

§ 371 Date: Feb. 3, 2023

Related U.S. Application Data
(60) Provisional application No. 63/060,545, filed on Aug. 3, 2020.
(60) Provisional application No. 63/111,561, filed on Nov. 9, 2020.

(51) Int. Cl. A61K 47/55

(52) U.S. Cl. CPC A61K 47/551; A61K 47/552

(57) ABSTRACT

Provided herein are methods and compositions using a target moiety linked to an active agent, in particular an antimicrobial agent. Such methods and compositions are useful in treating, e.g., microbial infections, such as antibiotic-resistant bacterial infections. Provided herein are methods and compositions using a target moiety linked to an active agent, in particular an antimicrobial agent. Such methods and compositions are useful in treating, e.g., microbial infections, such as antibiotic-resistant bacterial infections.

### CROSS-REFERENCE

This application claims the benefit of U.S. Provisional Applications Nos. 63/060,545 entitled “TARGETED DELIVERY OF THERAPEUTIC AGENTS”, filed Aug. 3, 2020, and 63/111,561 entitled “TARGETED DELIVERY OF THERAPEUTIC AGENTS”, filed Nov. 9, 2020, both of which applications are incorporated herein by reference.

### BACKGROUND

Drug-resistant microbial infections are a growing problem, especially antibiotic-resistant infections. New and effective treatments are needed.

### SUMMARY

In one aspect, provided herein are compositions.

In certain embodiments, provided is a composition comprising (i) a first moiety comprising a ligand that interacts with a cell that participates in infection healing to concentrate the first moiety on or in the cell, linked to (ii) a second moiety comprising an antimicrobial agent. In certain embodiments the ligand comprises a structure that is concentrated in the cell by passive diffusion. In certain embodiments the ligand comprises a ligand that interacts with a target structure of the cell. In certain embodiments the cell that participates in infection healing comprises an immune cell. In certain embodiments the immune cell comprises a lymphocyte, neutrophil, or monocyte/macrophage. In certain embodiments the immune cell comprises a lymphocyte comprising a T cell, a B cell, or a natural killer (NK) cell. In certain embodiments the immune cell comprises a neutrophil or a monocyte/macrophage. In certain embodiments the immune cell comprises a neutrophil. In certain embodiments the cell that participates in infection healing comprises a tissue repair cell. In certain embodiments the tissue repair cell comprises a fibroblast. In certain embodiments the target structure is a structure on the extracellular surface of a plasma membrane of the cell. In certain embodiments the target structure is a transmembrane moiety. In certain embodiments the transmembrane moiety is a transporter. In certain embodiments the transporter is a nutrient transporter. In certain embodiments the transporter comprises an amino acid transporter, a nucleic acid transporter, a carbohydrate transporter, an organic cation transporter, a fatty acid transporter, an antioxidant transporter, or a vitamin transporter. In certain embodiments the transporter is a carbohydrate transporter comprising a glucose transporter. In certain embodiments the glucose transporter comprises a GLUT1 (SLC2A1) or a GLUT3 (SLC2A3) transporter. In certain embodiments the transporter is an amino acid transporter. In certain embodiments the amino acid transporter comprises ATB^{0,+} (SLC6A14), b^{0,+}AT (SLC7A9), or xCT (SLC7A11). In certain embodiments the transporter is an organic cation transporter. In certain embodiments the organic cation transporter is OCNT1 (SLC22A4) or OCTN2 (SLC22A5). In certain embodiments the transporter is an antioxidant transporter or a vitamin transporter. In certain embodiments the transporter is an ascorbic acid transporter. In certain embodiments the ascorbic acid transporter comprises SVCT1, SVCT2 (SLC23A2), GLUT1 or GLUT3. In certain embodiments the ligand that interacts with the target moiety comprises ascorbic acid or an ascorbic acid derivative. In certain embodiments the target structure is increased in expression in response to infection. In certain embodiments the antimicrobial agent comprises an antibacterial agent, an antiviral agent, an antifungal agent, or an antiparasitic agent. In certain embodiments the antimicrobial agent has received regulatory approval. In certain embodiments the antimicrobial agent comprises an antibacterial agent. In certain embodiments the antibacterial agent comprises a quinolone, or a beta-lactam. In certain embodiments the quinolone comprises a fluoroquinolone. In certain embodiments the fluoroquinolone comprises ciprofloxacin, sitafloxacin, dalofloxacin, antofloxacin, levonadifloxacin, gemifloxacin, acorafloxacin, amifloxacin, avarofloxacin, balofloxacin, benofloxacin, besifloxacin, cadroflocacin, clinafloxacin, danofloxacin, ecenofloxacin, enoxacin, enrofloxacin, esafloxacin, finafloxacin, fleroxacin, gatifloxacin, grepafloxacin, irloxacin, lemefloxacin, levofloxacin, lomefloxacin, marbofloxacin, merafloxacin, motifloxacin, nadifloxacin, orbifloxacin, pazufloxacin, pefloxacin, pradofloxacin, premafloxacin, rosoxacin, rufloxacin, sarafloxacin, temafloxacin, trovafloxacin, ulifloxacin, vebufloxacin, or a combination thereof. In certain embodiments the antibiotic comprises a beta-lactam. In certain embodiments the beta-lactam comprises a carbapenem. In certain embodiments the first moiety comprises ascorbic acid or an ascorbic acid derivative. In certain embodiments the antibacterial agent comprises a beta-lactam. In certain embodiments the beta-lactam comprises a carbapenem. In certain embodiments the carbapenem comprises imipenem, meropenem, panipenem, biapenem, ertapenem, or tebipenem. In certain embodiments the first moiety comprises ascorbic acid or an ascorbic acid derivative. In certain embodiments the antimicrobial agent comprises an antiviral agent. In certain embodiments the antiviral agent comprises an adamantane antiviral, e.g., amantadine, rimantadine; an antiviral interferon, e.g., peginterferon alfa-2b, peginterferon alfa-2s, peginterferon alfa-2b; a chemokine receptor antagonist, e.g. maraviroc; an integrase strand transfer inhibitor, e.g. raltegravir, dolutegravir, elvitegravir; a neuraminidase inhibitor, e.g., zanamivir, oseltamivir, peramivir; a non-nucleoside reverse transcriptase inhibitor (NNRTI), e.g., etravirine, efavirenz, nevirapine, rilpivirine, doravirine, delavirdine; a non-structural protein 5A (Ns5A) inhibitor, e.g., daclatasivir; a nucleoside reverse transcriptase inhibitor (NRTI), e.g., kentecavir, lamivudine, adefovir, didanosine, tenofovir alafenamide, tenofovir, zidovudine, stavudine, emtricitabine, zalcitabine, telbivudine; a protease inhibitor, e.g., boceprevir, simeprevir, fosamprenavir, lopinavir, ritonavir, darunavir, telaprevir, tipranavir, atazanavir, nelfinavir, amprenavir, indinavir, saquinavir; a purine nucleoside, e.g., ribavirin, valacyclovir, acyclovir, famiciclovir, valganciclovir, ganciclovir, cidofovir. An antiviral booster is used in certain embodiments, e.g., ritonavir, cobicistat. In certain embodiments the antimicrobial agent comprises an antifungal agent. In certain embodiments the antifungal agent comprises amphotericin B; an azole derivative, e.g., ketoconazole, fluconazole, itraconazole, posaconazole, voriconazole; an echinocandin, e.g., anidulafungin, caspofungin, micafungin; flucytosine. In certain embodiments the antimicrobial agent comprises an antiparasitic agent. In certain embodiments the antiparasitic agent comprises an antimalarial agent. In certain embodiments the first and second moieties are linked covalently. In certain embodiments the covalent linkage comprises an ester, carbonate, amide, imine, acetal or ether linkage or a combination thereof. In certain embodiments the covalent linkage between the first and second moieties is a direct covalent linkage. In certain embodiments the covalent linkage between the first and second moieties is via a linkage moiety. In certain embodiments the covalent linkage is configured to be broken after the composition interacts with the cell that participates in infection healing. In certain embodiments the covalent linkage is configured to be broken in the presence of reactive oxygen species (ROS), in a low pH environment, or both. In certain embodiments the covalent linkage is hydrolytically stable. In certain embodiments the linkage comprises acetal-boronate. In certain embodiments the first and second moieties are linked noncovalently. In certain embodiments the first moiety comprises a first antimicrobial agent and the second moiety comprises a second antimicrobial agent, wherein the first and second antimicrobial agent are different. In certain embodiments the first antimicrobial agent comprises a fluoroquinolone, a tetracycline, or a macrolide. In certain embodiments the first moiety and the second moiety comprise areas of an antimicrobial agent.

In certain embodiments provided herein is a composition comprising an infection healing cell comprising an antimicrobial agent. In certain embodiments the antimicrobial agent comprises an antibacterial, an antiviral, an antifungal, or an antiparasitic agent. In certain embodiments the antimicrobial is present at a concentration of at least 1 ng/ml. In certain embodiments the infection healing is in an aqueous environment, and wherein the antimicrobial agent is present in the intracellular environment of the infection healing cell at a first concentration and in the extracellular aqueous environment at a second concentration, and wherein the ratio of first to second concentration is at least 2, 3, 4, 5, 6, 7, 8, 9, 10, 12, 15, 17, 20, 22, 25, 27, 30, 35, 40, 50, 60, 70, 80, 100. In certain embodiments the antimicrobial agent comprises an antibacterial agent. In certain embodiments the antibacterial agent comprises a fluoroquinolone or a beta-lactam. In certain embodiments the antimicrobial comprises a beta-lactam, a cephalosporin. In certain embodiments the antimicrobial is associated with the surface of the infection healing cell cell or an organelle of the infection healing cell, such as a lysosome, e.g., a lysosome in a neutrophil. In certain embodiments the antimicrobial is intracellular. In certain embodiments at least 50% of the antimicrobial is located in the cytosol. In certain embodiments the infection healing cell is capable of normal or substantially normal function. In certain embodiments the antimicrobial is linked to a moiety that interacts with a moiety of the infection healing cell.

In certain embodiments provided herein is a composition for treating a site of a drug-resistant bacterial infection comprising (i) an antibiotic specific for the drug-resistant bacteria linked to (ii) a ligand that targets infection healing cells at the site of infection or drawn to the site of infection.

In certain embodiments provided herein is a composition comprising (i) a first antimicrobial agent that is preferentially accumulated by one or more types of infection healing cells, linked to (ii) a second antimicrobial agent. In certain embodiments the first and second antimicrobial agents are different. In certain embodiments the first and second antimicrobial agents are the same type of antimicrobial agent. In certain embodiments the infection healing cell comprises an immune cell. In certain embodiments the immune cell comprises a phagocyte. In certain embodiments the infection healing cell comprises a wound repair cell. In certain embodiments the wound repair cell comprises a fibroblast. In certain embodiments the first antimicrobial agent comprises a macrolide. In certain embodiments the first antimicrobial agent comprises a fluoroquinolone. In certain embodiments the macrolide comprises azithromycin. In certain embodiments the second antimicrobial agent comprises a fluoroquinolone. In certain embodiments the second antimicrobial agent comprises a beta-lactam.

In certain embodiments provided herein is a composition comprising (i) a ligand that interacts with a moiety associated with an infection healing cell; (ii) a linker covalently linked to the ligand; and (iii) an antibiotic covalently linked to the ligand.

In certain embodiments provided herein is a pharmaceutical composition comprising a composition comprising an antimicrobial agent effective against one or more microbial agents linked to a ligand that interacts with a cell that participates in infection healing to concentrate the antimicrobial agent at the cell, and a pharmaceutically acceptable excipient.

In certain embodiments provided herein is a composition comprising (i) ascorbic acid or an ascorbic acid derivative linked to (ii) an antimicrobial agent. In certain embodiments the ascorbic acid or ascorbic acid derivative and the antimicrobial agent are linked noncovalently. In certain embodiments the ascorbic acid or ascorbic acid derivative and the antimicrobial agent are linked covalently. In certain embodiments the antimicrobial agent comprises an antibiotic. In certain embodiments the antibiotic is a fluoroquinolone or a beta-lactam. In certain embodiments the antibiotic comprises carbapenem. In certain embodiments the linker is hydrolytically stable but cleaved by reactive oxygen species (ROS). In certain embodiments the linker comprises acetal-boronate.

In certain embodiments provided herein is a composition comprising (i) a first antimicrobial agent that interacts with an infection healing cell in such a way as to increase the concentration of the antimicrobial agent at the infection healing cell, linked to (ii) a second antimicrobial agent. In certain embodiments the first and second antimicrobial agents are two of the same agent. In certain embodiments the first and second antimicrobial agents are different.

In certain embodiments provided herein is a composition comprising (i) a ligand targeting a target moiety associated with a natural killer (NK) or a T cell linked to (ii) a moiety comprising an antiviral agent.

In certain embodiments provided herein is a composition comprising (i) a ligand targeting a target moiety associated with a monocyte/macrophage linked to (ii) a moiety comprising an antifungal agent. In certain embodiments provided herein is a composition comprising (i) a first moiety linked to (ii) a second moiety; wherein the first and second moieties are linked via a linker comprising acetal-boronate.

In certain embodiments provided herein is a composition comprising (i) an infection healing cell comprising a membrane transporter for transporting a ligand across a cell membrane of the infection healing cell; (ii) a ligand or a derivative of the ligand, linked to an antimicrobial agent, wherein the ligand or ligand derivative is attached to the transporter, or is inside the infection healing cell.

In one aspect, provided herein are methods.

In certain embodiments provided is a method of accumulating an antimicrobial agent in a cell comprising (i) contacting the cell extracellularly with the antimicrobial agent linked to a ligand that interacts with a cell that participates in infection healing to concentrate the first ligand on or in the cell; (ii) allowing the antimicrobial agent linked to the ligand to accumulate in the cell. In certain embodiments the method further comprises (iii) cleaving the linkage between the ligand and the antimicrobial agent to release the agent in active form.

In certain embodiments provided herein is a method of delivering an antimicrobial agent to a site of an infection, mediated by one or more microbial agents, in an individual comprising (i) administering to the individual a composition comprising an antimicrobial agent linked to a ligand that interacts with an infection healing cell to concentrate the antimicrobial agent at the infection healing cell, wherein the infection healing cell is a cell that is present at the site of infection or that preferentially travels to the site of infection; and (ii) causing the antimicrobial agent to interact with the one or more microbial agents at the site of infection. In certain embodiments step (iii) comprises lysis of the infection healing cell. In certain embodiments at least one of the one or more microbial agents comprises an antibiotic-resistant bacterium.

In certain embodiments provided herein is a method of treating an infection caused by one or more microbial agents in an individual suffering from the infection comprising administering to the individual an effective amount of a composition comprising an antimicrobial agent effective against the one or more microbial agents linked to a ligand that interacts with an infection healing cell to concentrate the antimicrobial agent at the infection healing cell.

In certain embodiments provided is method of transporting an antimicrobial agent into a cell comprising contacting the cell with an effective amount of a composition comprising a ligand for a transporter in the plasma membrane of the cell linked to the antimicrobial agent under conditions wherein the ligand binds to the transporter and is carried into the cell along with the antimicrobial agent.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the structure of ascorbic acid

FIG. 2 shows the structure of 6-aminoascorbic acid

FIG. 3 shows the structure of 5-aminoascorbic acid

FIG. 4 shows one core structure of a carbapenem

FIG. 5 shows another core structure of a cabapenem

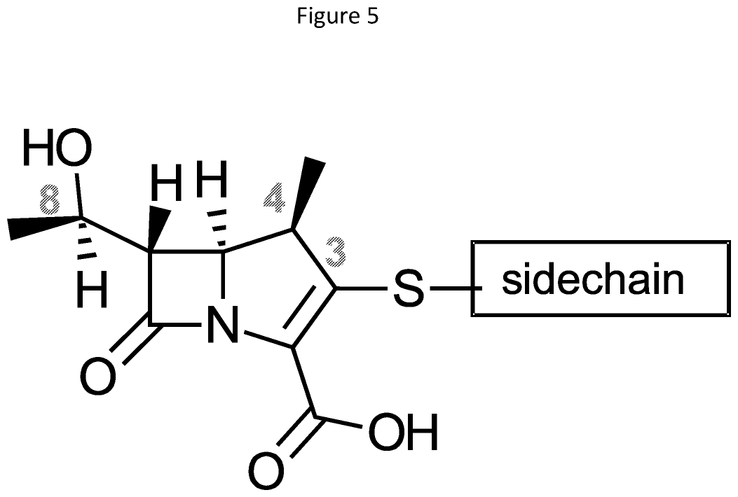

FIG. 6 shows another core structure of a carbapenem

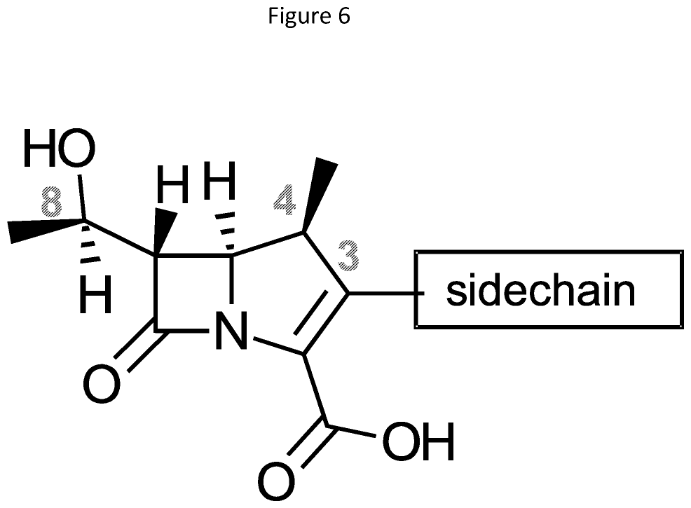

FIG. 7 shows the structure of imipenem

FIG. 8 shows the structure of meropenem

FIG. 9 shows the structure of panipenem

FIG. 10 shows the structure of biapenem

FIG. 11 shows the structure of ertapenem

FIG. 12 shows the structure of tebipenem

FIG. 13 shows structures of amino or imino groups of carbpenem-3 position sidechains linked with corresponding linkers to oxygen atom of 5- or 6-position of ascorbic acid

FIG. 14 shows structures of carboxy groups of carbapenem 3-position sidechains linked with corresponding linkers to oxygen atom of 5- or 6-position of ascorbic acid

FIG. 15 shows structures of carbapenem core 8-position oxygen linked with corresponding linkers to oxygen atom of 5- or 6-position of ascorbic acid

FIG. 16 shows structures of employing carbapenem N atom of carbapenem 3-position sidechain linked with corresponding linkers to N atom of 5- or 6-position corresponding aminoascorbic acid

FIG. 17 shows structures of carbapenem core 8-position oxygen linked with corresponding linkers to N atom of 5- or 6-position corresponding aminoascorbic acid

FIG. 18 shows ascorbic acid linked through 6-position to amine of a 3-position sidechain, as exemplified by meropenem (type L1-L6)

FIG. 19 shows compositions of type L1-L6 with ascorbic acid linked through 5-position to amine of a 3-position sidechain as exemplified by meropenem

FIG. 20 shows compositions of type L1-L6 with ascorbic acid linked through 6-position to imine of a 3-position sidechain as exemplified by panipenem

FIG. 21 shows compositions of type L1-L6 with ascorbic acid linked through 5-position to imine of a 3-position sidechain as exemplified by panipenem

FIG. 22 shows compositions of general types L7-L12 employing carboxy groups of carbapenem 3-position sidechains linked with corresponding linkers to oxygen atom of 5-position of ascorbic acid as exemplified by ertapenem

FIG. 23 shows compositions of type L13-L16 with ascorbic acid linked through 6-position to oxygen atom at 8-position of carbapenem core as exemplified by imipenem

FIG. 24 shows compositions of type L13-L16 with ascorbic acid linked through 5-position to oxygen atom at 8-position of carbapenem core as exemplified by imipenem

FIG. 25 shows compositions of type L17-L22 with 5-amino ascorbic acid linked through 5-position amine to amine of a 3-position sidechain as exemplified by meropenem

FIG. 26 shows compositions of type L17-L22 with ascorbic acid linked through 6-position to oxygen atom at 8-position of carbapenem core as exemplified by tebipenem

FIG. 27 shows structures of fluoroquinolone of core structure A or B

FIG. 28 shows structures of prodrugs consisting of ascorbic acid moieties, linkers and fluoroquinolone (amine group nitrogen linked fluoroquinolone structures)

FIG. 29 shows structures of prodrugs consisting of ascorbic acid moieties, linkers and fluoroquinolone (hydroxy group oxygen linked fluoroquinolone structures)

FIG. 30 shows structures of compositions comprising ascorbic acid linked through 6-position or 5-position to secondary aliphatic amine of a fluoroquinolone, for example, ciprofloxacin

FIG. 31 shows structures of compositions comprising ascorbic acid linked through 6-position or 5-position to primary aliphatic amine of a fluoroquinolone as exemplified by sitafloxacin.

FIG. 32 shows compositions comprising ascorbic acid linked through 6-position or 5-position to heteroaromatic amine of a fluoroquinolone as exemplified by dalofloxacin

FIG. 33 shows compositions comprising ascorbic acid linked through 6-position or 5-position to an aromatic amine of a fluoroquinolone as exemplified by antofloxacin

FIG. 34 shows compositions comprising ascorbic acid linked through 6-position or 5-position to a hydroxy group of a fluoroquinolone as exemplified by levonadifloxacin

FIG. 35 shows compositions comprising ascorbic acid linked through 6-position and 5-position to a primary amino group of a fluoroquinolone Core B as exemplified by Gemifloxacin.

FIG. 36 shows structures of ergothioneine and potential substitutions of the ergothioneine core

FIG. 37 shows examples of fluoroquinolone, as exemplified by ciproflaxin, linked with ergothioneine.

FIG. 38 shows examples of beta-lactam, as exemplified by meropenem, linked with ergothioneine

FIG. 39 shows examples of linkers comprising —C(O)O—C(R₁)(R₂)—, wherein R₁ and R₂ are independently selected from H, Me, Et, i-Pr, CH₂NH₂, CH₂NHMe, CH₂NHC(O)Me, CH₂NMeC(O)Me, CH₂NHMe, CH₂NMe₂, OMe. The linkers in this Figure are shown attached to a hydroxy on the targeting moiety and to a carboxy on a fluoroquinolone moiety, these are merely exemplary and do not limit the linkers.

FIG. 40 shows examples of linkers comprising —CH₂OC(O)O—C(R₁)(R₂)—, wherein R₁ and R₂ are independently selected from H, Me, Et, i-Pr, CH₂NH₂, CH₂NHMe, CH₂NHC(O)Me, CH₂NMeC(O)Me, CH₂NHMe, CH₂NMe₂. The linkers in this Figure are shown attached to a hydroxy on the targeting moiety and to a carboxy on a fluoroquinolone moiety, these are merely exemplary and do not limit the linkers.

FIG. 41 shows examples of linkers comprising —C(O)O—(C(R₁)(R₂))ₙ—, wherein n=2-5 and wherein R₁ and R₂ are independently selected from H, Me, Et, i-Pr, CH₂NH₂, CH₂NHMe, CH₂NHC(O)Me, CH₂NMeC(O)Me, CH₂NHMe, CH₂NMe₂, OH, OMe, OCH₂CH₂OH, and wherein R₁ and R₂ together may also represent carbonyl —C(O)—. The linkers in this Figure are shown attached to a hydroxy on the targeting moiety and to a carboxy on a fluoroquinolone moiety, these are merely exemplary and do not limit the linkers.

FIG. 42 shows examples of linkers comprising —C (O)(C(R₁)(R₂))ₚ(C(R₄)(R₅))ᵣ(C(O)—O—C(R₁)(R₂))ₛ—, wherein p=0-1, r=1-5, s=0-1, wherein R₁ and R₂ are independently selected from H, Me, CH₂NMe₂, OH, NH₂, and wherein R₁ and R₄ can be also connected to form saturated carbocyclic 3-6 membered ring, saturated heterocyclic 5-6 membered ring or 5-6 membered heteroaromatic ring containing 1-3 nitrogen atoms optionally substituted with NH₂, NHMe or NMe₂ group. The linkers in this Figure are shown attached to a hydroxy on the targeting moiety and to a carboxy on a fluoroquinolone moiety, these are merely exemplary and do not limit the linkers.

FIG. 43 shows examples of linkers comprising —(CH₂O)_{d}C(O)—(CH₂)ₑ(OCH₂CH₂)_{g}—, wherein d=0-1, e=0-2, and g=1-3. The linkers in this FIGURE are shown attached to a hydroxy on the targeting moiety and to a carboxy on a fluoroquinolone moiety, these are merely exemplary and do not limit the linkers.

FIG. 44 shows examples of linkers comprising —(C(R₁)(R₂))—ₙ, wherein n=0, 1, and R₁ and R₂ are independently selected from H, Me, Et, OMe, OEt, i-Pr, CH₂NH₂, CH₂NHMe, CH₂NHC(O)Me, CH₂NMeC(O)Me, CH₂NHMe, CH₂NMe₂, OCH₂CH₂NHMe, OCH₂CH₂NMe₂, and an amino substituted pyridine or imidazole ring, and wherein geminal R₁ and R₂ can be also connected to form saturated carbocyclic 3-6 membered ring, saturated heterocyclic 5-6 membered ring or 5-6 membered heteroaromatic ring containing 1-3 nitrogen atoms optionally substituted with NH₂, NHMe or NMe₂ group. The linkers in this FIGURE are shown attached to a hydroxy on the targeting moiety and to a carboxy on a fluoroquinolone moiety, these are merely exemplary and do not limit the linkers. The top left hand structure shows direct linkage with no intermediate linkage.

### DETAILED DESCRIPTION OF THE INVENTION

### Outline

I. Introduction
II. Target cells
   A Immune cells
   B. Tissue repair cells
III. Ligands
   A. Surface moieties
   B. Transmembrane moieties
IV. Antimicrobials
   A. Antibacterials (antibiotics)
      1. Classes of antibiotics
      2. Quinolones and fluoroquinolones
      3. Beta-lactams
   B. Antivirals
   C. Antifungals
   D. Antiparasitics
V. Linkage
   A. Covalent
   B. Noncovalent
VI. Conditions
   A. Infections
      1. General
      2. Drug-resistant infection
   B. Others
VI. Compositions
VII. Methods

### I. INTRODUCTION

The methods and compositions provided herein are used for targeted delivery of therapeutic agents to one or more sites of action. In certain embodiments, one or more therapeutic agents, e.g., drugs, may be targeted to be delivered at one or more sites of action, e.g., by a ligand that interacts with cells at the one or more sites to deliver a payload that includes the one or more therapeutic agents, e.g., drugs. The payload may be delivered intracellularly and/or extracellularly. The interaction may be any suitable interaction, such as with one or more transporters on the cells, with one or more agents released by or associated with the cells (e.g., reactive oxygen species, ROS), or any other suitable interaction that targets the one or more drugs to the location of the target cells, e.g., passive accumulation into the cell and/or organelle of the cell. In certain embodiments, antimicrobial drugs, such as antibiotics, antivirals, antifungals, or antiparasitics, can be targeted to particular sites using methods and compositions described herein, such as targeted to one or more sites of infection. In certain embodiments, provided herein are compositions that comprise one or more antimicrobial moieties linked to a ligand that is recognized by a transporter or other suitable moiety on a target cell; the target cell can be any suitable cell that serves to bring the antimicrobial to a site of action, such as a site of infection. Target cells can include immune cells, e.g., white blood cells such as lymphocytes, or, e.g., neutrophils. Target cells can also include cells that exist in areas that otherwise receive little or no blood supply, such as fibroblasts, which can be useful in targeting infections that otherwise would not receive an adequate dose of antimicrobial as well as e.g., for patients with compromised blood supply, such as diabetics. The linkage between the drug, e.g., antimicrobial, and the ligand can be any suitable linkage, e.g., a covalent linkage or a noncovalent linkage, so long as the drug or drugs linked to the ligand is rendered active for its intended purpose at the target site. In some cases the drug may remain linked to the ligand and remain active; in other cases the linkage is broken or modified, e.g., a covalent linkage may be cleaved chemically in any suitable manner. When the target cell comprises a transporter, suitable transporters may include those that have a relatively high concentration on cell surface of target cells so that one or more antimicrobials may be selectively increased in concentration at the site or sites that the target cells are located and/or preferentially migrate to, e.g., one or more sites of infection. Without being bound by theory, it is thought that the ligand interacts with its target in such a way as to deliver the payload (drug or drugs) to the target or in the vicinity of the target, for example, by being transported inside the target cell (intracellular delivery), including active and passive transport, by being delivered or converted in active form outside the target cell (extracellular delivery) or a combination thereof.

Methods and compositions provided herein may be used in any suitable situation in which it is desired to deliver a drug to a specific site of action, e.g., to cause a localized high concentration of the drug that would not be achievable, or would not be achievable safely, by systemic administration of the drug alone. This can be useful in a variety of situations. One such situation is treatment of infection, in some cases, an infection where the causative agent is drug-resistant. For convenience, methods and compositions will be described for targeting of drug-resistant bacterial infections but it will be appreciated that this is merely exemplary and that any suitable agent for localized treatment at the site of target cells may be used. In exemplary embodiments, an antibiotic or antibiotics are delivered to sites of drug-resistant infection by targeting immune cells or other infection healing cells that are concentrated at the site of infection. The antibiotic or antibiotics may be any suitable antibiotic, including available commercial antibiotics. One advantage of using available commercial antibiotics is that such agents have already been tested and approved. The compositions and method provided herein allow for localized high concentrations of the antibiotic or antibiotics to such a degree that even drug-resistant bacteria can be eliminated or their numbers greatly attenuated. In addition, compositions and methods provided herein allow production of localized concentrations of therapeutic agents, e.g., antibiotics that would not be achievable by conventional systemic administration, e.g., because of toxicity from the required doses if administered systemically. Thus, with or without drug resistance, therapeutically effective localized concentrations of a drug may be achieved that would otherwise be untenable. In the case of drug-resistant bacterial infections, targeted delivery of modified commercialized antibiotics using immune and/or other infection healing carrier cells to the site of infection increases the antibiotic concentration at the site of infection therefore improving clinical outcome and reversing resistance by restoring the susceptibility of the bacteria to the antibiotic.

### II. TARGET CELLS

In general, the compositions and methods provided herein are used to target one or more cell types in order to concentrate the active agent, e.g., antimicrobial, at those cells and/or at the site or sites where those cells are active. In some cases, the targeting ligand of a composition targets a moiety or moieties that is in greater abundance in a particular cell type, and/or in a particular situation, such as at the site where the cell type is active. Thus, in these cases, the composition is preferentially associated with the cells that possess the target moiety in greatest abundance. Alternatively or additionally, the compositions used are inactive or only partially active until the active agent, e.g., antimicrobial, such as an antibiotic, is cleaved from the rest of the composition (the rest of the composition comprising, typically, a second moiety such as a targeting ligand and, generally, a linker) and are activated only, or mostly, in target cells, for example cells where reactive oxygen species (ROS) are released, or in an acidic environment, such as in lysosomes, and/or at target sites; in this case, a composition may be widely distributed but only active in certain environments where the active moiety is cleaved from the rest of the composition.

In certain embodiments, compositions are targeted to a site or sites of infection. In some cases, targeting is achieved by a composition that comprises a ligand that interacts with a target moiety of a cell or cells that participate in infection healing. Such a cell may be an immune cell that is involved in actively fighting infection, and/or it may be a tissue repair cell that is involved in tissue repair and rebuilding at the site of infection.

By targeting a composition comprising an active agent at the site of infection, e.g., either by targeting moieties on cells at the infection site, or releasing the agent at the infection site, or both, it is possible to achieve high concentrations of the active agent, e.g., antimicrobial such as an antibiotic, preferentially at the infection site, while concentrations in the rest of the body are lower; thus, therapeutic levels may be reached at the infection site that would otherwise produce side effects if distributed throughout the body. Hence, in certain embodiments provided herein are methods and compositions for achieving targeted concentrations of active agent, e.g., an antimicrobial such as an antibiotic, at a specific site of action, e.g., an infection site, where the targeted concentration is a concentration that is therapeutic at the site of action, e.g., infection site, but that would be toxic if distributed throughout the body. It will be appreciated that if the kinetics of movement of the composition to the site of action is relatively rapid, e.g., into infection healing cells, compared to the kinetics of release of therapeutic moiety from the linker, then suitable concentrations may be achieved at the site of action, without large accumulation systemically.

### A. Immune Cells

In certain embodiments, the target cell or cells is/are immune cells. Any suitable type of immune cell, such as immune cells that are attracted to a site of infection may be targeted.

In certain cases, a novel immuno-approach is used to target an active agent, such as an antimicrobial, e.g., an antibiotic, by engaging the body's own infection fighting cells (immune cells) for targeted delivery of immuno-active agent, such as immuno-antimicrobials, to the site of infection.

Central to the immune system's ability to mobilize a response to an invading pathogen, toxin, or allergen, is its ability to distinguish self from non-self. The host uses both innate and adaptive mechanisms to detect and eliminate pathogenic microbes, and both of these mechanisms include self-non-self discrimination. The immune system uses many potent mechanisms that have the ability to destroy a broad range of microbial cells and to clear a broad range of both toxic and allergenic substances.

The immune system is comprised of cells and proteins that work together to provide defense against infection. These cells and proteins do not form a single organ like the heart or liver. Instead, the immune system is dispersed throughout the body to provide rapid responses to an infection. Cells travel through the bloodstream or in specialized vessels called lymphatics. Lymph nodes and the spleen provide structures that facilitate cell-to-cell communication.

The cells of the immune system can be categorized as lymphocytes (T-cells, B-cells and NK cells), neutrophils, eosinophils, basophils, and monocytes/macrophages. These are all types of white blood cells. The major proteins of the immune system are predominantly signaling proteins (often called cytokines), antibodies, and complement proteins.

Although all components of the immune system interact with each other, it is typical to consider two broad categories of immune responses: the innate immune system and the adaptive immune system. Innate immune responses are those that rely on cells that require no additional “training” to do their jobs. These cells include neutrophils, monocytes, natural killer (NK) cells and a set of proteins termed the complement proteins. Innate responses to infection occur rapidly and reliably. Adaptive immune responses involve T-cells and B-cells, two cell types that require “training” or education to learn not to attack our own cells. The advantages of the adaptive responses are their long-lived memory and the ability to adapt to new infections. Central to both categories of immune responses is the ability to distinguish foreign invaders (things that need to be attacked) from our own tissues, which need to be protected. Because of their ability to respond rapidly, the innate responses are usually the first to respond to an infection. This initial response serves to alert and trigger the adaptive response, which can take several days to fully activate.

The major phagocytic cells are neutrophils, monocytes and macrophages. These cells engulf pathogenic microbes and localize them in intracellular vacuoles, where they are exposed to toxic effector molecules, such as nitric oxide, superoxide, and degradative enzymes in an effort to destroy the invading organism.

Neutrophils or polymorphonuclear leukocytes (polys or PMN's) are the most numerous of all the types of white blood cells, making up about half or more of the total. They are also called granulocytes because they contain granules in their cytoplasm. They are found in the bloodstream and can migrate into sites of infection within a matter of minutes. They are the cells that leave the bloodstream and accumulate in the tissues during the first few hours of an infection and are responsible for the formation of “pus.” Their major role is to ingest bacteria or fungi and kill them. Their killing strategy relies on ingesting the infecting organisms in specialized packets of cell membrane that then fuse with other parts of the neutrophil that contain toxic chemicals that kill the microorganisms. They have little role in the defense against viruses.

Monocytes are closely related to neutrophils and are found circulating in the bloodstream. They make up 5-10 percent of the white blood cells. They also line the walls of blood vessels in organs like the liver and spleen. Here they capture microorganisms in the blood as the microorganisms pass by. When monocytes leave the bloodstream and enter the tissues, they change shape and size and become macrophages. Macrophages are essential for killing fungi and the class of bacteria to which tuberculosis belongs (mycobacteria). Like neutrophils, macrophages ingest microbes and deliver toxic chemicals directly to the foreign invader to kill it.

T-cells (sometimes called T-lymphocytes) are another type of immune cell. T-cells directly attack cells infected with viruses, and they also act as regulators of the immune system. An important aspect of the T-cell arm of the immune system is to recognize host cells that are infected by viruses, intracellular bacteria, or other intracellular parasites. T cells have evolved an elegant mechanism that recognizes foreign antigens together with self-antigens as a molecular complex. T-cells perform the actual destruction of infected cells. Killer T-cells protect the body from certain bacteria and viruses that have the ability to survive and even reproduce within the body's own cells. The killer cell must migrate to the site of infection and directly bind to its target to ensure its destruction.

B-cells (sometimes called B-lymphocytes) are specialized cells of the immune system whose major function is to produce antibodies (also called immunoglobulins or gamma-globulins). When B-cells encounter foreign material (antigens), they respond by maturing into another cell type called plasma cells. B-cells can also mature into memory cells, which allows a rapid response if the same infection is encountered again. Plasma cells are the mature cells that actually produce the antibodies.

Natural killer (NK) cells are so named because they easily kill cells infected with viruses. NK cells kill virus-infected cells by injecting it with a killer portion of chemicals. They are particularly important in the defense against herpes viruses.

Exemplary immune cells that may be targeted are one or more types of white blood cells, e.g., lymphocytes, such as T-cells, B-cells, and natural killer (NK) cells; neutrophils; and/or monocytes/macrophages. An immune cell can be targeted by using a ligand for a target moiety of the immune cell that is expressed on the cell, in some cases in greater concentration/quantity on the immune cell than on other cells and/or is expressed in greater concentration/quantity on the immune cell when the cell is activated, e.g., at the site of infection or elsewhere the cell is activated, and/or has greater activity when the immune cell is activated. Additionally or alternatively, an immune cell can be targeted by preferential cleavage of a linker at the site of immune cells fighting infection; for example, a linker that is cleaved by ROS will release active agent preferentially in immune cells, such as phagocytic cells, that fight infection at least in part by an oxidative burst that destroys microbial cells. In some cases, a ligand is used that leads to passive accumulation of an attached therapeutic agent in the cell, without necessarily targeting a particular moiety of the cell. Passive accumulation can occur when, e.g., an agent is altered within the cell or cellular compartment so that its ability to escape the cell or compartment is decreased. In certain embodiments, an antimicrobial used in a composition is basic, e.g., weakly basic, so that when it crosses into an acidic environment, e.g., lysosome, it becomes protonated, losing a charge, and can't exit the acidic environment back across a membrane, or exits only slowly. An example is the basic macrolide antibiotic azithromycin, which, without being bound by theory, appears to move into neutrophils and further into lysosomes of the neutrophils by passive diffusion; once in the lysosome, the basic azithromycin is protonated, losing charge, and then cannot move, or move only slowly, back out of the lysosomes, effectively trapping the azithromycin there; a specific transporter does not appear to be involved but rather conversion to a non-mobile form within the lysosome after passive diffusion.

In certain embodiments, the target cell comprises a lymphocyte, such as a T cell, a B cell, or a NK cell, or a phagocytic cell, such as a neutrophil, a monocyte, or a macrophage. In certain embodiments, the target cell comprises a neutrophil.

Specific target moieties of various immune cells that can be targeted by a ligand that interacts with such target moieties are described in section III, Ligands.

### B. Tissue Repair Cells

During and after infection eradication, damage may be done to surrounding tissue. Tissue repair cells, such as connective tissue cells, then proliferate and repair and replace the damaged tissue.

Tissue repair cells, e.g., connective tissue cells, that may be targeted in the compositions and method of the inventions include dendritic cells and fibroblasts. Although not part of the immune system, a fibroblast is a type of biological cell that synthesizes the extracellular matrix and collagen, produces the structural framework (stroma) for body tissues. Fibroblasts play an important role in tissue repair, a primary site of a trauma induced site of infection. Thus, in certain embodiments, the target cell comprises a fibroblast. Fibroblasts are the workhorse of the most important tissue that holds the human body together—connective tissue. Connective tissue joins and supports all other tissues, including the parenchymal tissues of organs. This connective tissue is made of fibroblasts widely-spaced in a vast extracellular matrix (ECM) of fibrous proteins and gelatinous ground substance. Fibroblasts produce the ECM's structural proteins and play various additional roles in ECM maintenance and reabsorption, tissue repair, inflammation, angiogenesis, cancer progression, and in physiological as well as pathological tissue fibrosis. Fibroblasts are ubiquitous mesenchymal cells derived from the embryonic mesoderm tissue, and they are not terminally differentiated. They can be activated by a variety of chemical signals that promote proliferation and cellular differentiation to form myofibroblasts with an up-regulated rate of matrix production. Ancillary to these various biological roles, fibroblasts produce and respond to a broad array of paracrine and autocrine signals, such as cytokines and growth factors.

The ground substance of ECM is a hydrated gel of proteo-glycans that is interspersed among the structural proteins. The ground substance forms a final pathway for nutrient flow beyond the reach of blood vessel transport into tissues as well as a pathway for intercellular communication. This cell-free medium forms an avenue for cell migration of immune cells, fibroblasts, and myofibroblasts. Fibroblasts have a pivotal role in tissue repair in response to tissue injury. First and foremost, fibroblasts respond to tissue repair by proliferating and by chemotaxing to the sites of tissue injury to rebuild the ECM as a scaffold for tissue regeneration. Fibroblast to myofibroblast transitioning enables the contraction of the matrix to seal an open wound in the event of the loss of tissue. Fibroblasts serve roles in inflammation and immune cell recruitment to sites of tissue injury. Furthermore, fibroblasts produce and are responsive to many inflammatory cytokines. Fibroblasts are responsive to cytokines such as TGFβ1, IL-1β, interleukin-6 (IL-6), IL-13, IL-33 as well as prostaglandins and leukotrienes. Fibroblasts are stimulated chemically by inflammatory agents to differentiate into myofibroblasts that have a greatly up-regulated rate of matrix production (discussed in more detail below). In turn fibroblasts produce and secrete cytokines such as TGFβ1, IL-1β, IL-33, CXC, and CC chemokines, as well as reactive oxygen species. These factors allow fibroblasts to assist in the activation and migration of resident immune cells such as macrophages. Moreover, the recruitment of non-resident immune cells is facilitated by the fibroblast-mediated production and maintenance of the relatively spacious, non-solid ground substance of the extracellular matrix, which plays an important role as a thoroughfare for the extravasation of immune cells into connective tissue. These tools endow fibroblast roles in chemical (non-specific) and cell-mediated immunity, acute and chronic inflammation, and inflammation resolution. Fibroblasts can contribute to chronic inflammation, and reciprocally, inflammatory cytokines promote fibroblast to myofibroblast transition, facilitating fibrosis. Furthermore, fibroblasts are chemotactic and can migrate and accumulate in new areas in response to secreted cytokines, a behavior well characterized in the tissue repair response after tissue injury. Fibroblasts are not a terminally differentiated cell type and retain the potential to be activated for differentiation into subtypes of fibroblast-like cells. Myofibroblasts are rarely found in healthy human physiology; they become vastly up-regulated after injury and play a critical role in the tissue repair response.

Targeting fibroblasts or other tissue repair cells also has the advantage that these cells can be in areas where perfusion is low, either as part of normal physiology, such as at the otitis media or in gingivitis, and/or as a result of a pathological condition such as cystic fibrosis or diabetes. In such a case, methods and compositions provided herein provide means whereby an active agent, such as an antimicrobial, e.g., an antibiotic, can be accumulated even with poor circulation, because when the composition does reach the site with, e.g., a fibroblast, it is retained there, thus allowing the active agent to accumulate at the site. An example is the use of azithromycin as a ligand; fibroblasts are thought to be a site of azithromycin accumulation; thus, azithromycin, itself an antibiotic, can be used as a ligand to target another therapeutic moiety, e.g., another antibiotic, to cells such as fibroblasts. In addition, an active agent such as an antimicrobial can be accumulated in such cells and not exposed to conditions that inactivate the agent, then released unchanged into the circulation, thus, in effect, extending the half-life of the agent, e.g., antimicrobial. In some cases, a particular subset of fibroblasts, may be targeted, such as one of the subsets of alveolar fibroblasts, such as myofibroblasts, lipofibroblasts, matrixfibroblasts, and alveolar niche cells. In some cases, a particular subset of fibroblasts, e.g., a subset that is activated in a pathological condition (e.g., fibroblasts that have undergone further differentiation), such as infection, may be targeted, e.g., myofibroblasts.

Specific target moieties of various tissue repairing cells, e.g., fibroblasts, that can be targeted by a ligand that interacts with such target moieties are described in section III, Ligands.

### III. LIGANDS

The methods and compositions provided herein involve the use of a composition comprising a ligand that interacts with a target moiety of a cell, e.g., a cell that participates in infection healing. As used herein, “interacts with” generally includes that the ligand associates with the target moiety in such a way as to bring the overall composition into contact with the target cell and to either bind the composition to the cell (generally though not necessarily through noncovalent interactions) or to move the composition into the cell, e.g., via a transporter targeted by the ligand or, e.g., passively into the cell and/or its organelles.

### A. Ligand Targeting Surface Moieties

In some cases, a ligand is used that targets a surface moiety on a target cell and that associates the composition comprising the target moiety and active agent, e.g., antimicrobial, with the extracellular surface of the cell, thus concentrating the active agent, e.g., antimicrobial, at the site of the cell. It can be beneficial if the surface moiety, e.g., protein, be expressed in greater concentration on the target cell than in other cells of the body and/or have greater activity during infection.

In embodiments in which the target cell is an immune cell, it can be possible to target specific types of immune cells using a composition whose ligand interacts with (e.g., binds with) cell surface markers specific for that type of immune cells. So long as such an interaction does not alter, or does not substantially alter, e.g., beneficially alters, the function of the immune cells, such targeting may be used. As is known in the art, different types of immune cells may be distinguished by different cell surface markers, typically cluster of differentiation (CD) markers. Thus, for example, the CD45 marker can be used to target leukocytes, though not necessarily one particular type of leukocytes; a composition would comprise a ligand that binds to the CD45 marker. Another example is that the CD8 marker may be used to target cytotoxic T cells, where it is preferentially expressed, and a composition would comprise a ligand that binds to the CD8 marker. Similarly, a CD4 marker can be used to target helper T cells, where it is preferentially expressed; such targeting can be useful, e.g., in compositions and methods to target viral infections, especially HIV infections. Other markers targeting other types of immune cells, and their appropriate ligands, will be apparent to those of skill in the art. It will be appreciated that a marker need not be absolutely specific to one type of cell, so long as it is present in the desired cell type in sufficient abundance to concentrate the active agent, e.g., antimicrobial, at the cell type (e.g., even if it binds to other cells than the desired cell, for example at lower concentrations).

### B. Ligands Targeting Transmembrane Moieties

In certain embodiments, compositions and methods of the invention utilize a composition comprising a ligand that targets a transmembrane moiety, e.g., a transmembrane moiety of the plasma membrane and/or a transmembrane membrane of an organelle. Particularly useful are ligands targeting transporters, because these can be used to move the desired composition comprising an active agent, e.g., an antimicrobial, to the intracellular compartment. In some cases the active agent, e.g., antimicrobial, exerts its effect intracellularly, e.g., an antibiotic to combat an intracellular bacterial infection. Alternatively or additionally, the active agent, e.g., antimicrobial, exerts its effect extracellularly, e.g., upon lysis or other disruption of the cell membrane of the target cell, such as when an antibiotic is used to combat an extracellular bacterial infection.

### 1. Ligands Targeting Transporters

In the case where a ligand targets a transporter, any suitable transporter may be targeted. In general, it is desirable that the transporter be expressed in the targeted cells, e.g., immune cells, for example, at a higher level than other cells and/or has greater activity in the targeted cells, so that the agent is taken up preferentially by immune cells, especially when activated, e.g., during infection Immune cells express subsets of transporters to fulfil their metabolic needs. Certain uptake transporters, that are overexpressed in stimulated immune cells, are suitable as a target for a ligand. In certain embodiments, the target transporter is a transporter, such as an ascorbic acid transporter, whose expression may increase in certain immune cells, such as white blood cells, e.g., neutrophils, when they are activated to fight an infection. The ligand can be a ligand for the transporter, such as ascorbic acid or an ascorbic acid derivative, and can be attached to the therapeutic agent, such as an antimicrobial agent, e.g., antibiotic, such as a beta-lactam or a fluoroquinolone.

Cells require nutrients as the building blocks for the synthesis of macromolecules (DNA, RNA, proteins, and lipids) and as the carbon source for generation of metabolic energy. These nutrients include glucose, amino acids, fatty acids, vitamins, and micronutrients such as trace elements. Most of these nutrients are hydrophilic and do not permeate easily across the plasma membrane in mammalian cells. Uptake of hydrophilic nutrients into cells requires specific transporters in the plasma membrane. Transporters, also known as carriers or permeases, bind a solute at one side of the membrane and deliver it to the other side. Upon stimulation, immune cells express or overexpress a specific subset of transporters such as the glucose transporter GLUT1 (SLC2A1), GLUT3 (SLC2A3), amino acid transporter ATB^{0,+} (SLC6A14), b^{0,+}AT (SLC7A9), xCT (SLC7A11), organic cation transporter OCNT1 (SLC22A4), OCTN2 (SLC22A5) and ascorbic acid transporter SVCT1 (SLC23A1) and SVCT2 (SLC23A2). Targeting these transporters offers a strategy to increase the concentration of antibiotics in the immune cells for targeted delivery, but this area has not received much attention. If the molecular and structural determinants that guide substrate recognition of a transporter are known, it is possible to decorate an antimicrobial with these substrate recognizing elements to make it a substrate for the transporter. This can be achieved by a prodrug approach, hybrid molecules or including the design elements into the antimicrobial itself. The substrate recognizing elements and the antimicrobial can be separated by a linker.

Thus, in certain embodiments, the ligand targets and interacts with a transporter comprising a nutrient transporter. As used herein, the term “nutrient” includes substances necessary or useful for proper cell function, and can include any suitable substance, such as amino acids, a nucleic acid, carbohydrates, organic cations, fatty acids, antioxidants, and/or vitamins. In certain embodiments, the ligand targets a carbohydrate transporter, such as a glucose transporter or a mannose transporter. Exemplary glucose transporters include a GLUT1 (SLC2A1) or a GLUT3 (SLC2A3) transporter. In certain embodiments, the ligand targets an amino acid transporter. Exemplary amino acid transporters include ATB^{0,+} (SLC6A14), b^{0,+}AT (SLC7A9), or xCT (SLC7A11). In certain embodiments, the ligand targets a transporter that is an organic cation transporter. Exemplary organic acid transporters include OCNT1 (SLC22A4) or OCTN2 (SLC22A5). Another ligand transporter includes transporters for ergothioneine. In certain embodiments the ligand targets a transporter that is an antioxidant transporter or a vitamin transporter, such as an ascorbic acid transporter. Exemplary ascorbic acid transporters include SVCT1, SVCT2 (SLC23A2), GLUT1 or GLUT3.

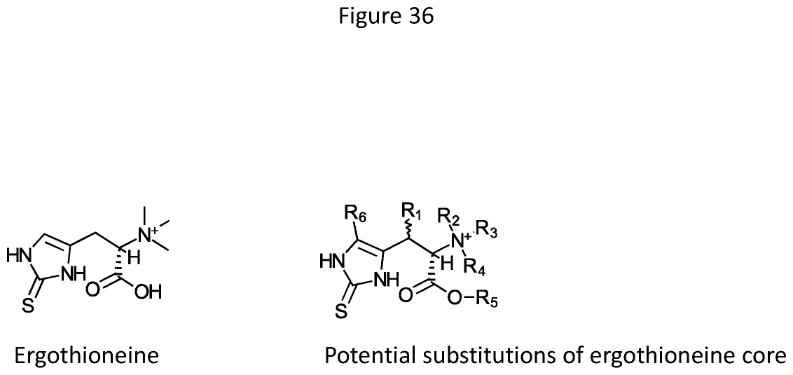

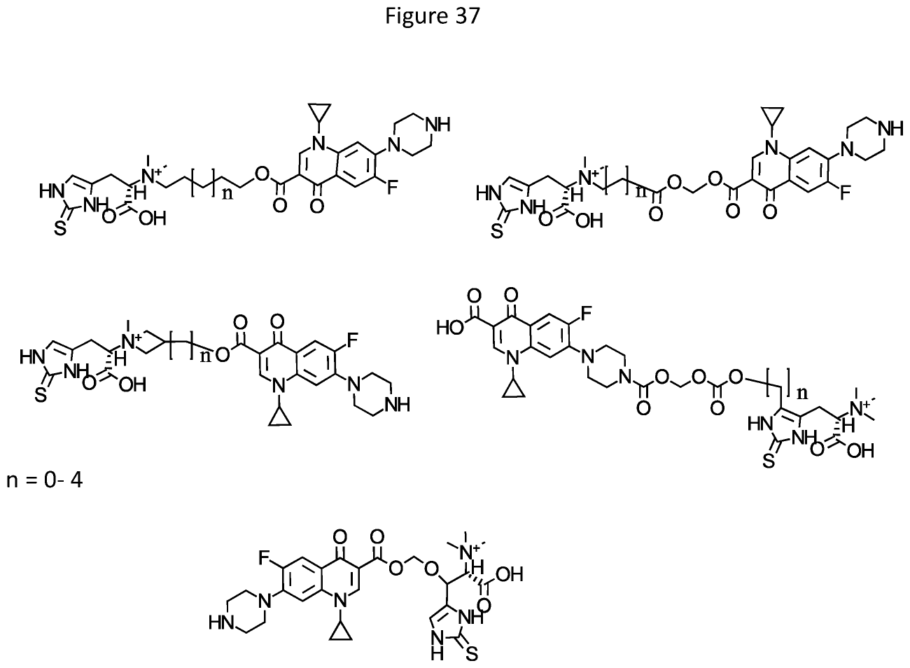

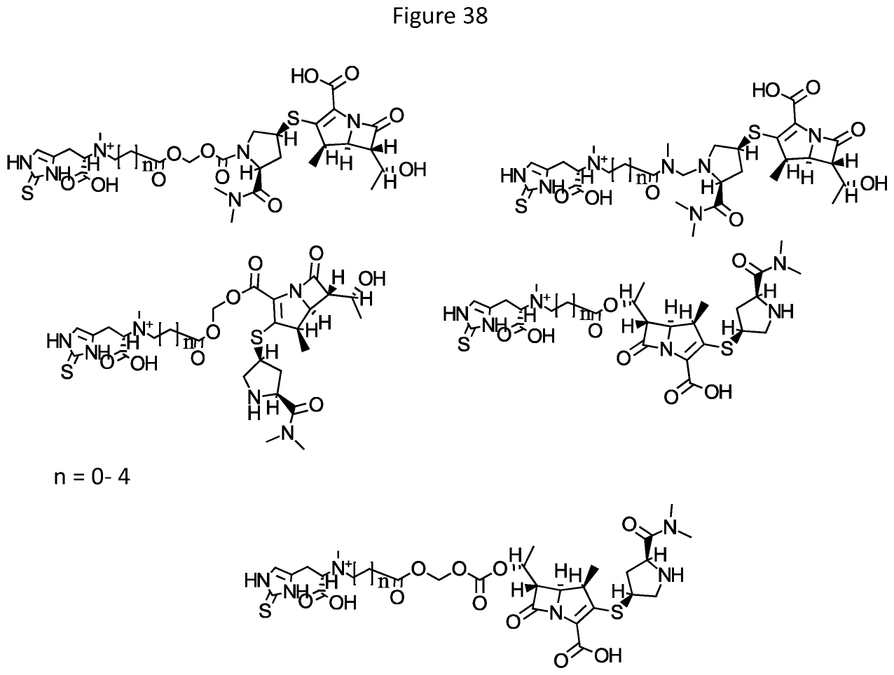

In embodiments where the ligand targets an ascorbic acid transporter, such as SVCT1, SVCT2 (SLC23A2), GLUT1 or GLUT3, the ligand can be ascorbic acid, a derivative thereof, dehydroascorbic acid, or a derivative thereof. Another useful transporter or transporters are those for ergothioneine, a vitamin-like compound that is known to be particularly suited for quenching ROS, in particular singlet oxygen. It has been shown to be more efficient in that respect than glutathione. See FIGS. 36, 37, and 38.

Ascorbic acid transporters are especially useful in targeting cells, such as neutrophils, that increase expression of such transporters in response to infection in order to provide antioxidant protection to the surrounding tissue from its own oxidative burst; thus, they can be used as specific targets to preferentially direct a composition comprising a ligand such as ascorbic acid, dehydroascorbic acid, or derivatives of either, and an active agent, such as an antimicrobial, e.g., an antibiotic, to concentrate the active agent at the cell, e.g. neutrophil and, ultimately, at the site of infection. Any suitable form of ascorbic acid, dehydroascorbic acid, or their derivatives or analogs may be used so long as it is taken up, e.g., by a transporter and transported into the cell (along with its attached active agent). Suitable ascorbic acid derivatives include 5-aminoascorbic acid and 6-aminoascorbic acid. Any suitable linkage between the ascorbic acid and the active agent, e.g., antimicrobial, may be used, including a direct linkage between the ascorbic acid and the active agent, e.g., antimicrobial or an indirect linkage via an intermediate moiety (e.g., acetal-boronate, as described more fully elsewhere herein). Exemplary linkages that can be used with ascorbic acid, dehydroascorbic acid, or derivatives, are described in section V, Linkages.

Further useful ligands include glucose or a glucose derivative, DHA, mannose, galactose, amino acid, amino acid derivatives, carnitine, colistin, cephaloridine, ergothioneine, cytarabine, nucleotide, cytidine or derivatives, gemcitabine, amino acid or derivative, cystine, cationic amino acids, cystathionine, glutamate.

### IV. ANTIMICROBIALS

In embodiments of compositions and methods provided herein in which one or both of the moieties of a composition comprise an antimicrobial agent, any suitable antimicrobial may be used. An antibiotic can be used as a therapeutic moiety. In some cases, an antibiotic can be used as a targeting moiety. Thus, in certain embodiments, both moieties are antibiotics, and a first antibiotic functions as a ligand for targeting a second, active antibiotic linked to the first antibiotic. Though in some cases the first antibiotic may also have therapeutic properties for treating a condition, it is used at least as a targeting ligand in these compositions. In certain embodiments, an antimicrobial agent comprises an antibacterial (as used herein, generally synonymous with “antibiotic”) agent. In certain embodiments, the antimicrobial comprises an antiviral agent. In certain embodiments, the antimicrobial comprises an antifungal agent. In certain embodiments, the antimicrobial comprises an antiparasitic agent. In certain embodiments, an antibiotic is used that is a broad spectrum antibiotic, such as fluoroquinolones and beta-lactams (e.g., cephalosporins, cephamycins, oxamazines, monocarbams, monobactams and carbapenems).

A. Antibacterials (antibiotics)

In embodiments in which an antibacterial (antibiotic) is used as a therapeutic agent linked to a ligand, any suitable antibiotic may be used; in some cases, two antibiotic moieties are linked, with one as targeting antbiotic (ligand) and the other as active antibiotic. It is desirable that the antibiotic, e.g., active antibiotic, used be effective against the bacterial agent or agents causing an infection. In certain embodiments, the antibiotic is a broad-spectrum antibiotic. In certain embodiments, the antibiotic is most effective against Gram-negative bacteria. In certain embodiments, the antibiotic is most effective against Gram-positive bacteria. In certain embodiments, the antibiotic is one that is effective, or has some effect, on antibiotic-resistant bacteria, which can depend on the particular type of antibiotic-resistant bacteria.

### 1. Classes of Antibiotics

If an antibiotic is used, any suitable antibiotic may be used. Thus, in certain embodiments, the antibiotic is a beta-lactam, such as a carbapenem, a cephalosporin, a monobactam, or a penicillin; an aminoglycoside; a quinolone such as a fluoroquinolone; a glycopeptide or lipoglycopeptide, such as vancomycin; a macrolide, such as erythromycin or azithromycin; an oxazolidinone, such as linezolid or tedizolid; a polypeptide; a rifamycin; a sulfonamide, a streptogramin such as quinupristin or dalfopristin; chloramphenicol or tiamphenicol; clindamycin; daptomycin; fosfomycin; lefamulin; metronidazole; mupirocin; nitrofurantoin; or tigecycline; or a combination thereof

### 2. Fluoroquinolones

In certain embodiments, the antibiotic is a fluoroquinolone. Any suitable fluoroquinolone may be used. Exemplary fluoroquinolones useful in embodiments of the methods and compositions herein include ciprofloxacin, sitafloxacin, delafloxacin, antofloxacin, levonadifloxacin, and gemifloxacin. Other fluoroquinolones that may be useful in compositions and methods provided herein include acorafloxacin, amifloxacin, avarofloxacin, balofloxacin, benofloxacin, besifloxacin, cadroflocacin, clinafloxacin, danofloxacin, ecenofloxacin, enoxacin, enrofloxacin, esafloxacin, finafloxacin, fleroxacin, gatifloxacin, grepafloxacin, irloxacin, lemefloxacin, levofloxacin, lomefloxacin, marbofloxacin, merafloxacin, motifloxacin, nadifloxacin, orbifloxacin, pazufloxacin, pefloxacin, pradofloxacin, premafloxacin, rosoxacin, rufloxacin, sarafloxacin, temafloxacin, trovafloxacin, ulifloxacin, vebufloxacin. Any suitable ligand and linkage of the ligand to the fluoroquinolone may be used; in certain embodiments, the ligand targets a transporter, for example a transporter that is expressed in greater concentrations and/or has greater activity in an infection healing cell, e.g., immune cell or tissue repair cell, that concentrates at the site of a bacterial infection, e.g., a neutrophil, than in other cells. One exemplary ligand is ascorbic acid or an ascorbic acid derivative. Specific examples of fluoroquinolones linked to ascorbic acid or ascorbic acid derivative are given in section VI, Compositions. Another example is a ligand that causes passive accumulation in an infection healing cell, such as an immune cell or tissue repair cell.

### 3. Beta-Lactams

In certain embodiments, the antibiotic is a beta-lactam. Any suitable beta-lactam may be used, e.g., a carbapenem, a cephalosporin, a monobactam, or a penicillin. In certain embodiments, the beta-lactam is a carbapenem, such as imipenem, meropenem, panipenem, biapenem, ertapenem, or tebipenem. In certain embodiments, the beta-lactam is a cephalosporin, such as a third- or fourth-generation cephalosporin. In certain embodiments, the beta-lactam is a penicillin, such as an aminopenicillin, e.g., ampicillin, amoxicillin; an antipseudomonal penicillin, e.g., carbenicillin, piperacillin, ticarcillin; a natural penicillin, e.g., penicillin G, procaine penicillin G, penicillin V, benzathine; a penicillinase resistant penicillin, e.g., oxacillin, dicloxacillin, nafcillin. In certain embodiments, the beta-lactam is a monobactam, such as aztreonam, tigemonam, carumonam, nocardicin A, tabtoxin. In certain embodiments, a beta-lactam antibiotic is used in combination with a beta-lactamase inhibitor, for example, a beta-lactamase inhibitor that is itself linked to a ligand specific for a target moiety on a desired cell, either alone or in combination with a beta-lactam.

Any suitable ligand and linkage of the ligand to the beta-lactam may be used; in certain embodiments, the ligand targets a transporter, for example a transporter that is expressed in greater concentrations and/or has greater activity in an infection healing cell, e.g., an immune cell or tissue healing cell, that is concentrated at the site of a bacterial infection, e.g., a neutrophil, than in other cells. One exemplary ligand is ascorbic acid or an ascorbic acid derivative. Specific examples of beta-lactams (carbapenems) linked to ascorbic acid or ascorbic acid derivative are given in section VI, Compositions. Another example is a ligand that causes passive accumulation in an infection healing cell, such as an immune cell or tissue repair cell.

### B. Antivirals

If an antiviral agent is used, any suitable antiviral may be used. Thus, in certain embodiments, the antiviral is an adamantane antiviral, e.g., amantadine, rimantadine; an antiviral interferon, e.g., peginterferon alfa-2b, peginterferon alfa-2s, peginterferon alfa-2b; a chemokine receptor antagonist, e.g. maraviroc; an integrase strand transfer inhibitor, e.g. raltegravir, dolutegravir, elvitegravir; a neuraminidase inhibitor, e.g., zanamivir, oseltamivir, peramivir; a non-nucleoside reverse transcriptase inhibitor (NNRTI), e.g., etravirine, efavirenz, nevirapine, rilpivirine, doravirine, delavirdine; a non-structural protein 5A (Ns5A) inhibitor, e.g., daclatasivir; a nucleoside reverse transcriptase inhibitor (NRTI), e.g., kentecavir, lamivudine, adefovir, didanosine, tenofovir alafenamide, tenofovir, zidovudine, stavudine, emtricitabine, zalcitabine, telbivudine; a protease inhibitor, e.g., boceprevir, simeprevir, fosamprenavir, lopinavir, ritonavir, darunavir, telaprevir, tipranavir, atazanavir, nelfinavir, amprenavir, indinavir, saquinavir; a purine nucleoside, e.g., ribavirin, valacyclovir, acyclovir, famiciclovir, valganciclovir, ganciclovir, cidofovir. An antiviral booster is used in certain embodiments, e.g., ritonavir, cobicistat. In certain embodiments, more than one antiviral may be used; the antivirals may be attached to the same ligand, to different ligands, or a combination thereof. Examples of combination antivirals, where one or more of the antivirals are attached to one or more ligands, include emtricitabine/rilpivirine/tenofovir; abacavir/dolutegravir/lamivudine; emtricitabine/rilpivirine/tenofovir alafenamide; sofosbuvir/velpatasvir; cobicistat/darunavir/emtricitabine/tenofovir alafenamide; emtricitabine/tenofovir; bictegravir/emtricitabine/tenofovir alafenamide; cobicistat/elvitegravir/emtricitabine/tenofovir alafenamide; dasabuvir/ombitasivir/paritaprevir/ritonavir; dolutegravir/rilpivirine; cmtricitabine/tenofovir alafenamide; lamivudine/zidovudine/cobicistat/darunavir; emtricitabine/tenofovir; emtricitabine/lopinavir/ritonavir/tenofovir; emtricitabine/nelfinavir/tenofovir; lamivudine/tenofovir; doravirine/lamivudine/etnofovir; atazanavir/cobicistat; efavirenz/lamivudine/tenofovir; ombitasvir/paritaprevir/ritonavir; sofosbuvir/velpatasvir/voxilaprevir.

Any suitable ligand and linkage of the ligand to the antiviral may be used; in certain embodiments, the ligand targets a transporter, for example a transporter that is expressed in greater concentrations and/or has greater activity in an infection healing cell, e.g., an immune cell or tissue repair cell that is concentrated at a site of a viral infection, e.g., an NK cell or a T cell, than in other cells. In certain cases, e.g., when delivering one or more antivirals to combat an HIV infection, the ligand may be one that targets T-helper cells.

### C. Antifungals

If an antifungal agent is used, any suitable antifungal may be used. Thus, in certain embodiments, the antifungal is amphotericin B; an azole derivative, e.g., ketoconazole, fluconazole, itraconazole, posaconazole, voriconazole; an echinocandin, e.g., anidulafungin, caspofungin, micafungin; flucytosine. Combinations of antifungals may be used, either attached to the same ligand or to different ligands, e.g., flucytosine and amphotericin B, or flucytosine and an antifungal azole.

Any suitable ligand and linkage of the ligand to the antifungal may be used; in certain embodiments, the ligand targets a transporter, for example a transporter that is expressed in greater concentrations and/or has greater activity in an infection healing cell, such as an immune cell or tissue repair cell that is concentrated at a site of a viral infection, e.g., a monocyte/macrophage, than in other cells.

### D. Antiparasitics

If an antiparasitic agent is used, any suitable antiparasitic may be used. In certain embodiments, the antiparasitic agent is an antimalarial agent and, in some of these embodiments, the ligand is a ligand that targets red blood cells.

### V. LINKAGE

In general, the compositions and methods of the invention include a first moiety, such as a targeting ligand, linked to a second moiety, such as an antimicrobial. The first and second moiety are “linked,” as that term is used herein, if they are joined in a common structure that remains intact or substantially remains intact under conditions of use, except where intentionally designed to be cleaved under certain conditions during use. Thus, for example, in certain embodiments the two moieties are linked and remain linked in blood, and at least initially when interacting with a target moiety on a cell; in certain embodiments the moieties remain linked (e.g., if the active agent, such as an antimicrobial, remains active or substantially active in linked form) and in other embodiments the linkage between the moieties is cleaved (e.g., to release the active agent, such as intracellularly). The moieties are “linked” when they are joined in a common structure. In certain embodiments, the moieties are directly linked; that is, there is no intermediate between the moieties. In certain embodiments, the moieties are indirectly linked; that is, the moieties are joined via an intermediate linker. The linkage may be covalent or noncovalent.

### A. Covalent Linkage

In certain embodiments, moieties are linked via one or more covalent bonds. In some cases, a target ligand moiety is linked directly to an active agent moiety, e.g., an antimicrobial agent, via a covalent bond between a group on the target ligand moiety and a group on the active agent (e.g., antimicrobial agent) moiety. Various groups, as known in the art, may react and form a covalent bond. Any linkage may be used so long as it remains stable under conditions of use, except where intentionally designed to be cleaved under certain conditions during use. In embodiments in which the active agent remains active or substantially active, the linkage should be such that it does not substantially interfere with the activity of the active moiety, e.g., an antimicrobial agent.

In some cases, a target ligand moiety is linked indirectly to an active agent moiety, e.g., antimicrobial agent, via an intermediate linker. In certain embodiments, the linker forms a covalent bond with the target ligand moiety and another covalent bond with the active agent moiety, e.g., antimicrobial agent. In certain embodiments, the linker and covalent bond formed between the linker and the target ligand moiety and the active agent moiety is such that the linkage remains stable under certain conditions of use but is cleaved under other conditions; any suitable cleavage condition may be used, e.g., a condition that exists mainly or exclusively at or near the site where it is desired that the active agent, e.g., antimicrobial agent, be released. An example is the acetal boronate linker, described further in section VI, Compositions, which forms bonds that are stable to hydrolysis but that are cleaved by reactive oxygen species, e.g., at or near the site of an oxidative burst of a phagocytic cell, such as a neutrophil. In this way, an active agent, such as an antimicrobial agent, e.g., an antibiotic, can be kept bonded to a linker moiety, in some cases in such a manner as to be partially or completely inactive, and only released at the site where its activity is desired. This is useful to allow the agent to be administered systemically but only have the agent active at its site of action, which can allow higher concentrations of the active agent at its desired site of action than are achieved systemically. These concentrations at the site of action can be higher than those that, if achieved systemically, would be toxic. It will be appreciated that, when an intermediate linker is used that covalently bonds to a ligand and to an antimicrobial, any suitable combination of bond breakage may suffice; e.g., in some cases, only the bond between the linker and the antimicrobial is broken; in some cases, only the bond between the ligand and the linker is broken (e.g., when the antimicrobial retains substantial activity when attached to the linker); in some cases, both may be broken.

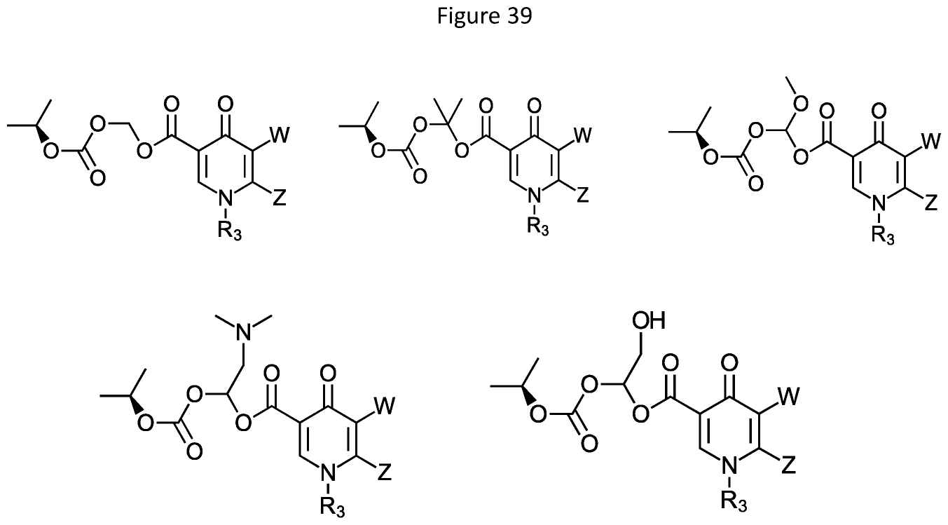

In certain embodiments, provided is a composition comprising a first moiety comprising a ligand that interacts with a cell that participates in infection healing to concentrate the first moiety on or in the cell, linked to a second moiety comprising a therapeutic agent, e.g., an antimicrobial agent, wherein the linker comprises —C(O)O—C(R₁)(R₂)—, wherein R₁ and R₂ are independently selected from H, Me, Et, i-Pr, CH₂NH₂, CH₂NHMe, CH₂NHC(O)Me, CH₂NMeC(O)Me, CH₂NHMe, CH₂NMe₂, OMe. In certain embodiments the linker comprises a linker as shown in FIG. 39; note that the linkers in FIG. 39 are shown attached to a hydroxy on the targeting moiety and to a carboxy on a fluoroquinolone moiety, these are merely exemplary and do not limit the linkers. The ligand may be any suitable ligand, such as a ligand described herein. An antimicrobial agent may be any suitable agent, such as one of those described herein, e.g., an antibiotic.

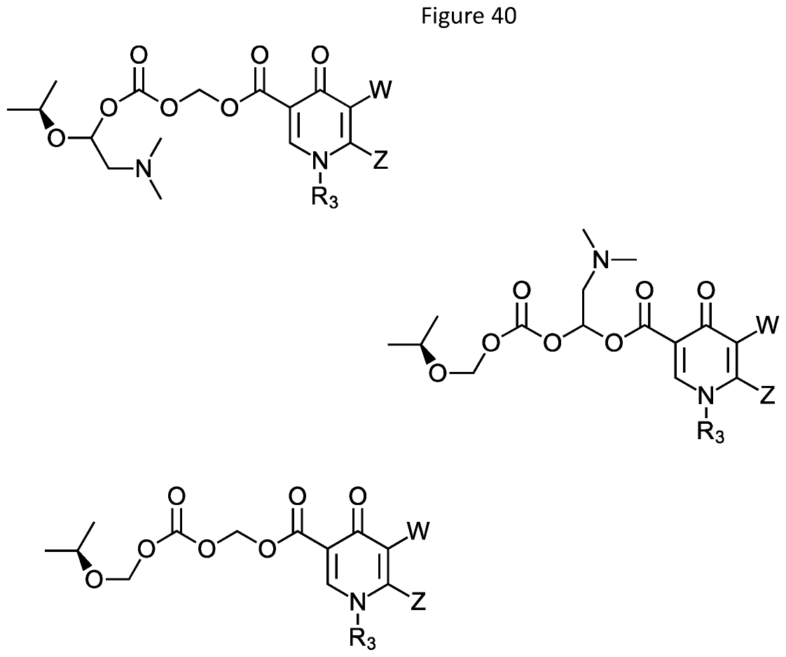

In certain embodiments, provided is a composition comprising a first moiety comprising a ligand that interacts with a cell that participates in infection healing to concentrate the first moiety on or in the cell, linked to a second moiety comprising a therapeutic agent, e.g., an antimicrobial agent, wherein the linker comprises —CH₂OC(O)O—C(R₁)(R₂)—, wherein R₁ and R₂ are independently selected from H, Me, Et, i-Pr, CH₂NH₂, CH₂NHMe, CH₂NHC(O)Me, CH₂NMeC(O)Me, CH₂NHMe, CH₂NMe₂. In certain embodiments the linker comprises a linker as shown in FIG. 40; note that the linkers in FIG. 40 are shown attached to a hydroxy on the targeting moiety and to a carboxy on a fluoroquinolone moiety, these are merely exemplary and do not limit the linkers. The ligand may be any suitable ligand, such as a ligand described herein. An antimicrobial agent may be any suitable agent, such as one of those described herein, e.g., an antibiotic.

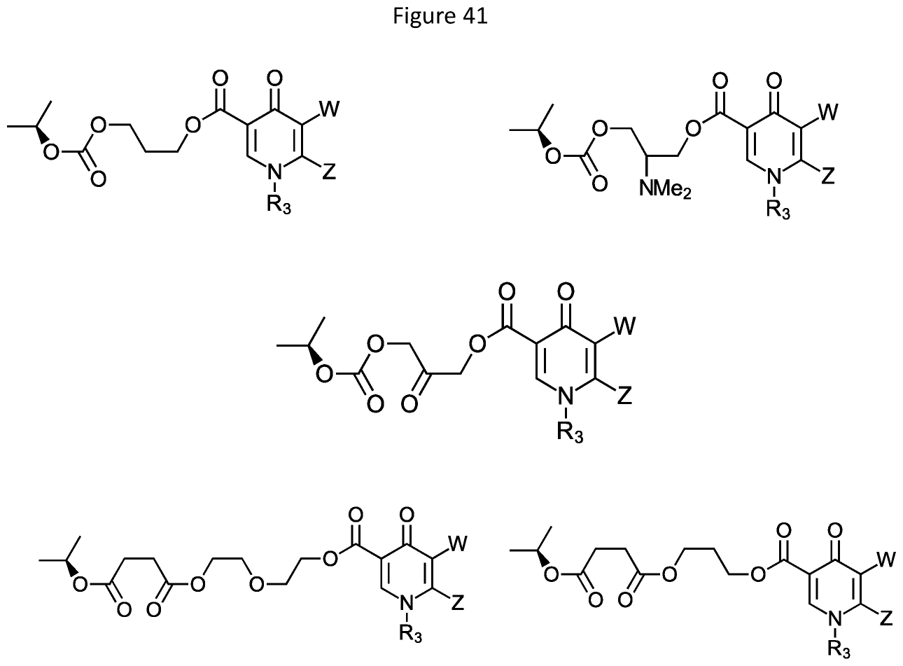

In certain embodiments, provided is a composition comprising a first moiety comprising a ligand that interacts with a cell that participates in infection healing to concentrate the first moiety on or in the cell, linked to a second moiety comprising a therapeutic agent, e.g., an antimicrobial agent, wherein the linker comprises —C(O)O—(C(R₁)(R₂))ₙ—, wherein n=2-5 and wherein R₁ and R₂ are independently selected from H, Me, Et, i-Pr, CH₂NH₂, CH₂NHMe, CH₂NHC(O)Me, CH₂NMeC(O)Me, CH₂NHMe, CH₂NMe₂, OH, OMe, OCH₂CH₂OH, and wherein R₁ and R₂ together may also represent carbonyl —C(O)—. In certain embodiments the linker comprises a linker as shown in FIG. 41; note that the linkers in FIG. 41 are shown attached to a hydroxy on the targeting moiety and to a carboxy on a fluoroquinolone moiety, these are merely exemplary and do not limit the linkers. The ligand may be any suitable ligand, such as a ligand described herein. An antimicrobial agent may be any suitable agent, such as one of those described herein, e.g., an antibiotic.

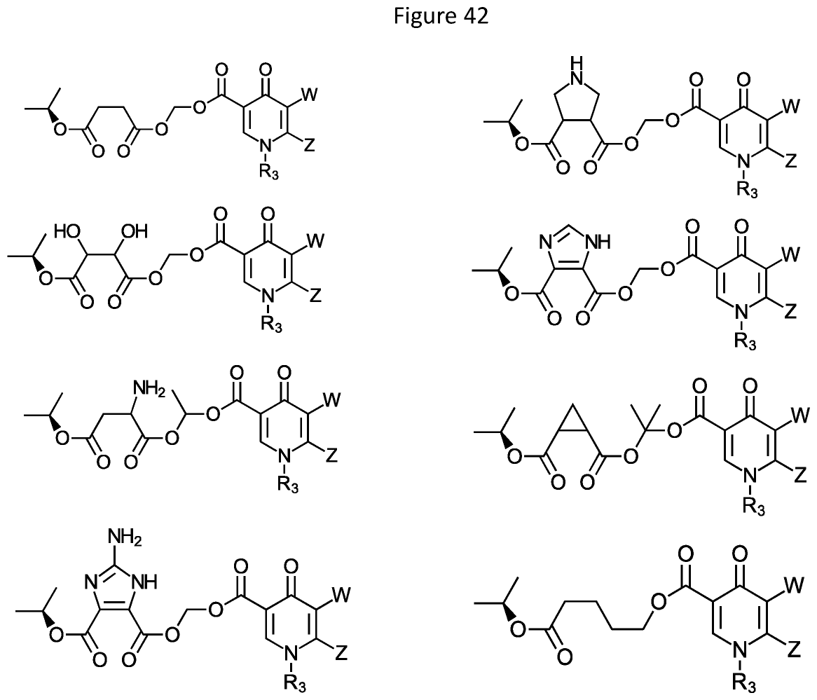

In certain embodiments, provided is a composition comprising a first moiety comprising a ligand that interacts with a cell that participates in infection healing to concentrate the first moiety on or in the cell, linked to a second moiety comprising a therapeutic agent, e.g., an antimicrobial agent, wherein the linker comprises —C(O)(C(R₁)(R₂))ₚ(C(R₄)(R₅))ᵣ(C(O)—O—C(R₁)(R₂))ₙ—, wherein p=0-1, r=1-5, s=0-1, wherein R₁ and R₂ are independently selected from H, Me, CH₂NMe₂, OH, NH₂, and wherein R₁ and R₄ can be also connected to form saturated carbocyclic 3-6 membered ring, saturated heterocyclic 5-6 membered ring or 5-6 membered heteroaromatic ring containing 1-3 nitrogen atoms optionally substituted with NH₂, NHMe or NMe₂ group. In certain embodiments the linker comprises a linker as shown in FIG. 42; note that the linkers in FIG. 42 are shown attached to a hydroxy on the targeting moiety and to a carboxy on a fluoroquinolone moiety, these are merely exemplary and do not limit the linkers. The ligand may be any suitable ligand, such as a ligand described herein. An antimicrobial agent may be any suitable agent, such as one of those described herein, e.g., an antibiotic.

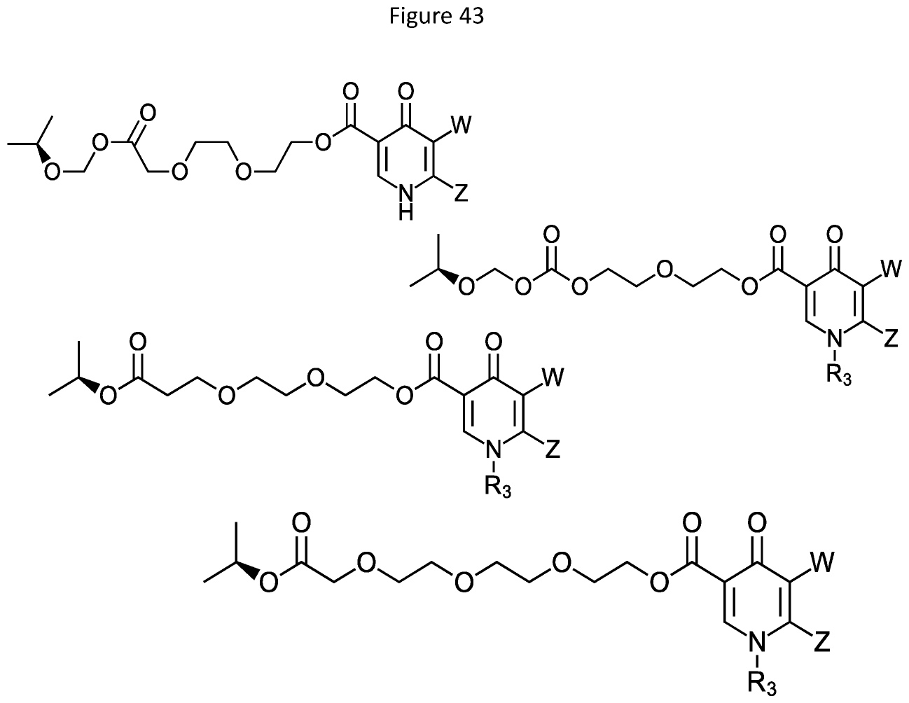

In certain embodiments, provided is a composition comprising a first moiety comprising a ligand that interacts with a cell that participates in infection healing to concentrate the first moiety on or in the cell, linked to a second moiety comprising a therapeutic agent, e.g., an antimicrobial agent, wherein the linker comprises —(CH₂O)_{d}C(O)—(CH2)ₑ(OCH2CH2)_{g}—, wherein d=0-1, e=0-2, and g=1-3. In certain embodiments the linker comprises a linker as shown in FIG. 43; note that the linkers in FIG. 43 are shown attached to a hydroxy on the targeting moiety and to a carboxy on a fluoroquinolone moiety, these are merely exemplary and do not limit the linkers. The ligand may be any suitable ligand, such as a ligand described herein. An antimicrobial agent may be any suitable agent, such as one of those described herein, e.g., an antibiotic.

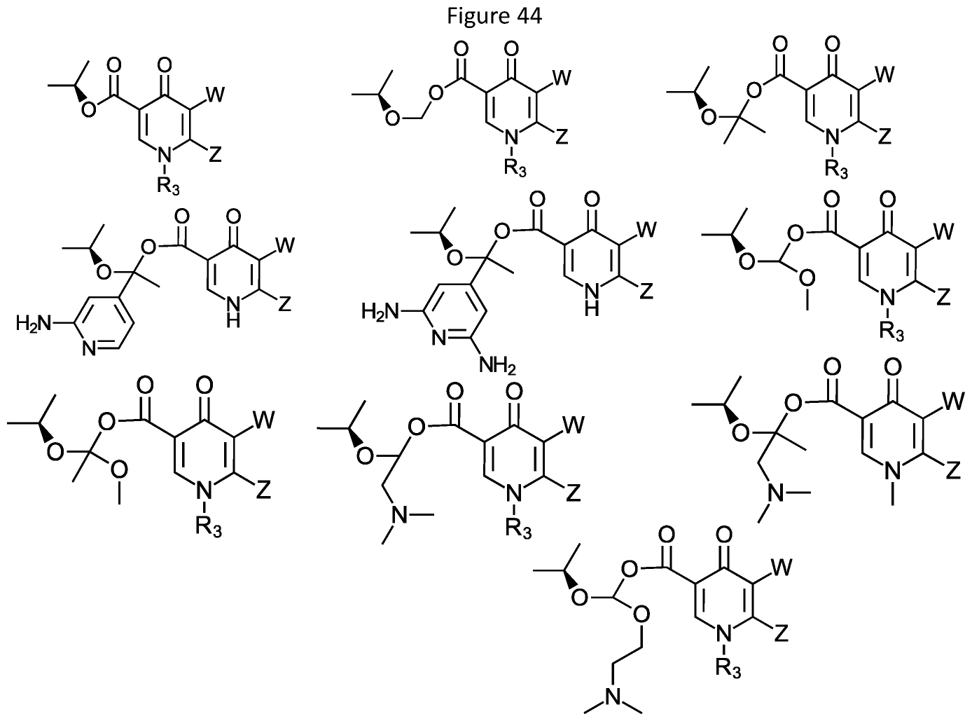

In certain embodiments, provided is a composition comprising a first moiety comprising a ligand that interacts with a cell that participates in infection healing to concentrate the first moiety on or in the cell, linked to a second moiety comprising a therapeutic agent, e.g., an antimicrobial agent, wherein the linker comprises —(C(R₁)(R₂))—ₙ, wherein n=0, 1, and R₁ and R₂ are independently selected from H, Me, Et, OMe, OEt, i-Pr, CH₂NH₂, CH₂NHMe, CH₂NHC(O)Me, CH₂NMeC(O)Me, CH₂NHMe, CH₂NMe₂, OCH₂CH₂NHMe, OCH₂CH₂NMe₂, and an amino substituted pyridine or imidazole ring, and wherein geminal R₁ and R₂ can be also connected to form saturated carbocyclic 3-6 membered ring, saturated heterocyclic 5-6 membered ring or 5-6 membered heteroaromatic ring containing 1-3 nitrogen atoms optionally substituted with NH₂, NHMe or NMe₂ group. In certain embodiments the linker comprises a linker as shown in FIG. 44; note that the linkers in FIG. 44 are shown attached to a hydroxy on the targeting moiety and to a carboxy on a fluoroquinolone moiety, these are merely exemplary and do not limit the linkers. Also note that the top left hand structure shows direct linkage with no intermediate linkage. The ligand may be any suitable ligand, such as a ligand described herein. An antimicrobial agent may be any suitable agent, such as one of those described herein, e.g., an antibiotic.

### B. Noncovalent

One or more bonds between moieties, whether direct or indirect, can also be noncovalent in certain embodiments. Noncovalent bonds include ionic bonds, hydrogen bonds, electrostatic interactions, Van der Waals interactions, and any other noncovalent bonds known in the art. So long as a noncovalent bond satisfies the desired conditions for use, any suitable noncovalent bond may be used.

### VI. CONDITIONS

In certain embodiments, the linked moieties of the invention are used in the treatment of one or more conditions. Any condition amenable to treatment by the linked moieties can be the subject of treatment.

### A. Infections

### 1. General

An infection occurs when a microbial agent invades an organism's body tissues, multiplying therein and causing damage to host tissues by the infectious agent and/or toxins they produce. A microbial infection can be caused by bacteria, viruses, fungi, or parasites, and the appropriate active agent, e.g., antibacterial (antibiotic), antiviral, antifungal, or antiparasitic can be targeted to one or more infection sites using the methods and compositions described herein.

When a bacterial infection is targeted, any suitable bacterial infection may be targeted with a suitable agent, including Gram-positive and Gram-negative bacteria.

### 2. Drug-Resistant Infection

Drug resistance is a growing global public health threat. According to the CDC report, more than 2.8 million antibiotic-resistant infections occur in the U.S. each year, resulting in about 35,000 deaths annually. Since 2004, only a handful of new antibiotics has been approved for the treatment of resistant Gram-negative bacteria. In 2014, a task force was established in the US to tackle this issue. Resistant bacteria have far reaching implications that may affect many advanced surgical procedures such as joint replacement as it results in a significant risk increase. US drug spending on antibiotics ranges between $8.4 to $10.6 billion annually. Globally, about 700,000 people die of drug-resistant infections every year with 13.5 billion dollars in annual financial losses due to hospital infections in the US and Europe alone. Drug resistance may push 28.3 million people into extreme poverty by 2050.

Rising drug resistance is a global crisis threatening our ability to treat common infectious diseases. Antibiotic resistance leads to higher medical costs, prolonged hospital stays, and increased mortality. Efficacy of antibiotics is directly related to achieving higher than the minimum inhibitory concentrations (MIC) at the site of infection. Resistance causes a sharp increase in MIC rendering lower efficacy. Hard to treat infections caused by resistant Gram-negative bacteria to include pneumonia, gonorrhea, and foodborne diseases. Based on CDC data, about 2.8 million infections with resistant bacteria were recorded in 2019 of those about 30% are patients with pneumonia. There is a need for new methods and compositions to deliver antimicrobials to treat infections.

In certain embodiments, a drug-resistant infection is treated using methods and compositions provided herein. For example, in certain embodiments, a drug-resistant bacterial infection is treated by targeting one or more antibiotics to the site of infection, for example, by linking the one or more antibiotics to a target ligand that associates with one or more types of immune cells that are drawn to the site of infection, and/or one or more tissue repair cells such as fibroblasts, so that the local concentration of the antibiotic or antibiotics is increased to a point that it overcomes the resistance of the bacteria causing the infection. Such local concentrations typically are high enough that, were they to be achieved by normal systemic administration of antibiotics, undesirable toxicity would result. Thus, an important aspect of certain embodiments provided herein is that very high local concentrations of the desired active agent can be achieved with little or no systemic toxicity.

In certain embodiments, Gram-negative highly resistant bacteria are targeted, such as, Pseudomonas Aeruginosa, Acinetobacter baumannii, Enterobacteriaceae, Neisseria gonorrhoeae, Campylobacter spp., Salmonellae spp., Shigella spp. These can be targeted using any suitable antibiotic, e.g., using commercially available antibiotics with known mechanism of action (MOA). Targeted delivery of commercially available antibiotics results in higher efficacy, lower toxicity and/or an improved probability of success for development and registration. In certain embodiments, an antibiotic is used that is a broad-spectrum antibiotic, such as fluoroquinolones and beta-lactams (e.g., cephalosporins, monobactams and carbapenem).

The mechanism of resistance for the fluoroquinolones and beta-lactams are known and well described in the literature. The approach of targeted delivery of commercially available antibiotics to the site of infection typically does not alter the MOA nor the mechanisms leading to resistance development. However, the approach reduces resistance because it increases the antibiotic concentration at the site of infection leading to a restoration of the susceptibility of the bacteria to the relevant antibiotic. This can apply to both intracellular and extracellular bacteria.

Thus, in certain embodiments, prodrugs of commercially available antibiotics with known safety and efficacy that target specific transporters on immune carrier cells to increase intracellular concentrations of antibiotics can be used. Antibiotic travels with the carrier cells and is present at the site of infection in active form; in some cases, the antibiotic is active even when linked to a target ligand, and in other cases the antibiotic is inactive or only partially active when linked to a target ligand and is released at the site of infection in its active form. This approach improves efficacy against both intracellular and extracellular pathogens. It can reduce systemic concentrations which may improve the overall safety profile of the therapeutic agent. Higher concentrations at the site of action eliminates highly resistant strains of bacteria and restore susceptibility of the bacteria to the antibiotic.

In certain embodiments, one of two classes of commercially available antibiotics that are considered the last resort to treat infections, for example of highly resistant Gram-negative bacteria, may be used. These include beta-lactams, in particular, carbapenems, and fluoroquinolones. See section VII, methods, for specific methods.

### B. Others

In some cases, a targeting ligand and an active agent are joined by a novel linker, such as acetal boronate, that releases the active agent only under certain conditions. As used herein, the term “acetal boronate” includes acetal-boronates comprising the substructure of (dihydroxymethyl)boranediol. In the case of acetal boronate, the linkage is cleaved, releasing active agent, in the presence of ROS. While this occurs at, e.g., a site of infection when a phagocytic cell produces an oxidative burst, such constructs are also useful in other conditions in which an oxidative condition occurs, such as cancer or cancer chemotherapy. Any condition that induces localized oxidative stress can be targeted by acetal boronate constructs, e.g., localized inflammation is often accompanied by large localized concentrations of ROS, and acetal boronate constructs can be used that comprise an anti-inflammatory agent that is inactive or partially active when linked by the acetal boronate to another moiety, but that is preferentially released in its active form at the site of inflammation due to the high concentration of ROS. The other moiety can be a target ligand, though in some cases this is not necessary, as the main area where the anti-microbial agent is released will be at the site of infection and the construct will otherwise remain inactive or only partially active. In this way, similar to antibiotics, a local concentration of an active agent can be achieved that, if it were systemic, would produce undesirable toxicity. Suitable inflammatory conditions include acute inflammation, e.g., due to injury, and/or chronic inflammation, e.g., due to an autoimmune disorder such as rheumatoid arthritis.

It will be appreciated that any suitable linker that is configured to be cleaved under conditions that are prevalent at a desired site of action and, in some cases, not prevalent systemically can be used. In certain cases, a composition may be accumulated intracellularly quickly enough that, even though the linker is also cleaved systemically, due to kinetics of uptake it is cleaved primarily after the composition has entered the cell, avoiding high systemic concentrations of the therapeutic moiety.

### VI. COMPOSITIONS

Provided herein are compositions.

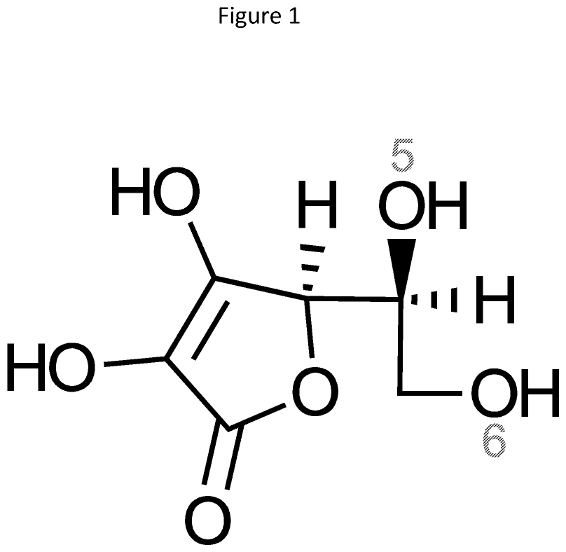

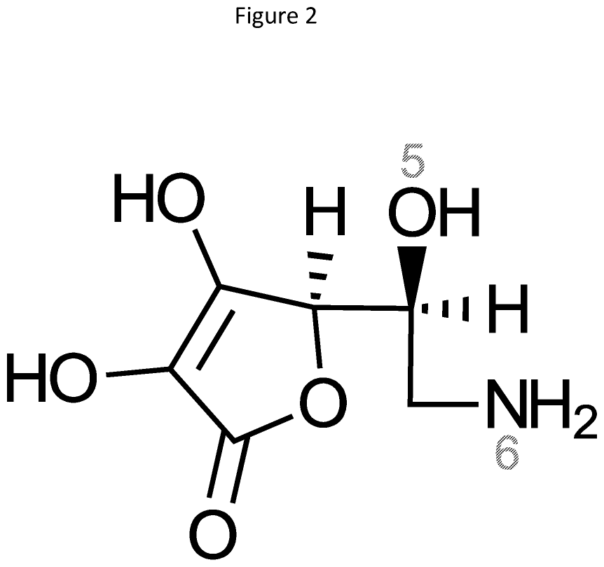

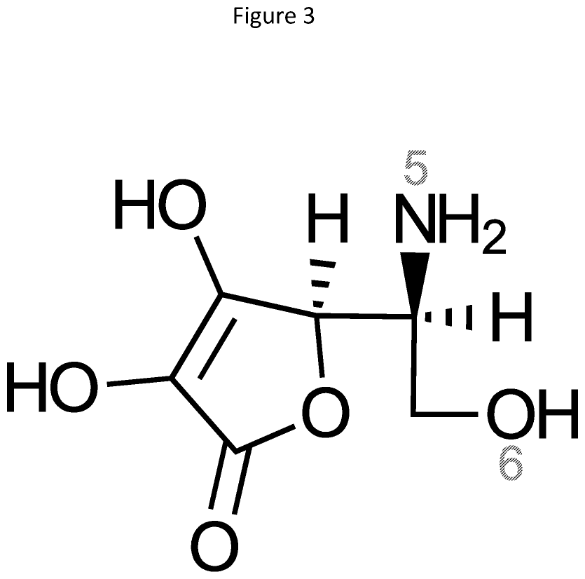

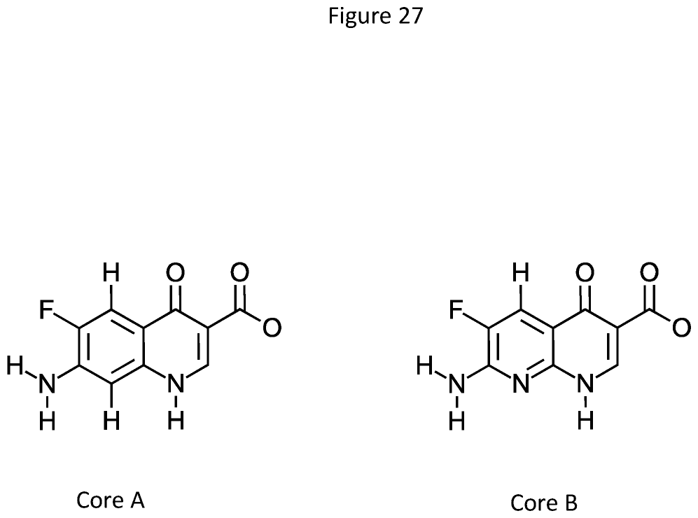

In certain embodiments, provided herein are compositions comprising a first moiety that interacts with a target cell of interest, e.g., a cell or type of cell to be targeted by the composition, in such a way as to increase its concentration on or in the cell, and a second moiety that comprises an active agent, e.g., a therapeutic agent, such as a therapeutic agent to be delivered to the cell of interest and/or to the environment of the cell of interest. Thus, for example, provided herein is a composition comprising (i) a first moiety comprising a ligand that interacts with a cell that participates in infection healing to concentrate the first moiety on or in the cell linked to (ii) a second moiety comprising an antimicrobial agent. In certain embodiments the ligand comprises a structure that is concentrated in the cell by passive diffusion. In certain embodiments, the ligand comprises a ligand that interacts with a target structure of the cell. For example, the ligand may target, e.g., bind to or interact with, any suitable target moiety (structure) such as those described elsewhere herein; the antimicrobial agent may be any suitable antimicrobial agent such as those described elsewhere herein; and the linkage may be any suitable linkage such as those described elsewhere herein. In certain embodiments, the cell that participates in infection healing can be an immune cell; in some cases the immune cell comprises a lymphocyte, neutrophil, or monocyte/macrophage; for example, the immune cell may comprise a lymphocyte such as a T cell, B cell, or a natural killer (NK) cell; or the immune cell may comprise a neutrophil or monocyte/macrophage; in some cases the immune cell is a neutrophil. Neutrophils are especially desirable because they constitute a large portion of circulating immune cells, they congregate at sites of infection, and transporters useful as targets for target ligands can be upregulated as a result of infection. In certain embodiments, the ligand is a structure that accumulates in an organelle of a target cell, e.g., in lysosomes of neutrophils or other infection healing cells, e.g., a tissue repair cell such as a fibroblast. In certain embodiments, the infection healing cell is a tissue repair cell; in certain embodiments the tissue repair cell comprises a fibroblast. In certain embodiments, the ligand interacts with a target moiety (structure) expressed by the cell that participates in infection healing that is a moiety of the extracellular surface of a plasma membrane of the cell; any suitable surface moiety that serves to preferentially target the desired cell may be targeted by the ligand, so long at the binding of the ligand to the target moiety does not interfere, or does not substantially interfere, with the normal function of the cell. Exemplary moieties of the extracellular surface of the cell that can be targeted by a ligand of the composition are described elsewhere herein. In certain embodiments, the ligand interacts with a target moiety (structure) expressed by a cell that participates in infection healing that is a transmembrane moiety. In certain embodiments, a transporter may be the target moiety. Any suitable transporter that serves to preferentially target the desired cell may be targeted by the ligand, so long at the binding of the ligand to the transporter and, typically, its subsequent transport (together with attached antimicrobial) to the intracellular space does not interfere, or does not substantially negatively interfere, with the normal function of the cell. Thus, any suitable ligand may be used with a given transporter so long as it fulfills these criteria; in some cases, the ligand is the normal physiological substance transported by the transporter; in other cases it is a derivative of the normal physiological substance or an analog or other similar structure. It is particularly desirable if the transport function of the transporter is increased in response to infection, e.g., in the infection healing cell such as an immune cell or tissue repair cell. Glucose and ascorbic acid transporters are examples of such transporters. In some cases the ligand targets a transporter that is a nutrient transporter, such as an amino acid transporter, a nucleic acid transporter, a carbohydrate transporter, an organic cation transporter, a fatty acid transporter, an antioxidant transporter, and/or a vitamin transporter. In certain cases the ligand targets a transporter that is a carbohydrate transporter, e.g., a glucose transporter, such as a GLUT1 (SLC2A1) or a GLUT3 (SLC2A3) transporter. In certain embodiments the transporter is a mannose transporter. In certain cases the ligand targets a transporter that is an amino acid transporter, such as ATB^{0,+} (SLC6A14), b^{0,+}AT (SLC7A9), and/or xCT (SLC7A11). In certain cases, the ligand targets a transporter that is an organic cation transporter, such as OCNT1 (SLC22A4) or OCTN2 (SLC22A5). In certain cases, the ligand targets an antioxidant transporter or a vitamin transporter such as an ascorbic acid transporter, e.g. SVCT1, SVCT2 (SLC23A2), GLUT1 and/or GLUT3; in certain instances, such as an ascorbic acid transporter, the substance transported is both a vitamin (at least in humans and guinea pigs) and an antioxidant. Thus, in certain cases the ligand is ascorbic acid or an ascorbic acid derivative such as 5- or 6-aminoascorbic acid; dehydroascorbic acid, or a derivative thereof. Ascorbic acid is shown in FIG. 1; an antimicrobial may be linked, directly or indirectly, at the 5-hydroxyl position or at the 6-hydroxyl position. 6-aminoascorbic acid is shown in FIG. 2; an antimicrobial may be linked, directly or indirectly, at the 5-hydroxyl position or at the 6-amino position. 5-amino ascorbic acid is shown in FIG. 3; an antimicrobial may be linked, directly or indirectly, at the 6-hydroxyl position or at the 5-amino position. Specific compositions comprising ascorbic acid or an ascorbic acid derivative are described further below. In certain embodiments, the ligand, e.g., ligand interacting with a target moiety on the cell, is itself an antibiotic, or part of an antibiotic, such as a fluoroquinolone, a quinolone, a naphtyridinone, a tetracycline, or a macrolide; thus, in these cases the composition comprises a first moiety that is an antibiotic or antibiotic derivative and the second moiety is an antimicrobial, which can also be an antibiotic—either the same type of antibiotic as the first moiety or a different antibiotic. The antimicrobial agent (second moiety) can be any suitable antimicrobial agent, such as an antibiotic, antiviral, antifungal, or antiparasitic agent, e.g., as described elsewhere herein. In certain cases, the antimicrobial agent is one that has received regulatory approval. In certain embodiments, the antimicrobial agent comprises an antibiotic; exemplary antibiotics are described in section IVA. In certain embodiments, the antibiotic is a fluoroquinolone or a beta-lactam; e.g., a fluoroquinolone with a core structure such as shown in FIG. 27 and/or a fluoroquinolone as described in section IVA2, or, e.g., a beta-lactam such as a carbapenem, e.g., imipenem, meropenem, panipenem, biapenem, ertapenem, doripenem, or tebipenem, a cephalosporin, a monobactam, e.g., aztreonam, tigemonam, nocardicin A, tabtoxin, or a penicillin, e.g., an aminopenicillin, e.g., ampicillin, amoxicillin; an antipseudomonal penicillin, e.g., carbenicillin, piperacillin, ticarcillin; a natural penicillin, e.g., penicillin G, procaine penicillin G, penicillin V, benzathine; a penicillinase resistant penicillin, e.g., oxacillin, dicloxacillin, nafcillin. In certain embodiments, the composition further comprises a beta-lactamase inhibitor, either as part of the ligand-link-antibiotic construct, or as a separate construct, e.g., a targeting ligand linked to a beta-lactamase inhibitor in similar manner to the constructs of target ligand linked to antimicrobial. In certain embodiments, the composition comprises ascorbic acid or a derivative or dehydroascorbic acid or a derivative, linked to a fluoroquinolone; specific exemplary compositions are described further, below. In certain embodiments, the composition comprises ascorbic acid or a derivative or dehydroascorbic acid or a derivative, linked to a beta-lactam, such as a carbapenem, e.g., imipenem, meropenem, panipenem, biapenem, ertapenem, or tebipenem; specific compositions are described further, below. In certain embodiments, the antimicrobial agent comprises an antiviral agent; any suitable antiviral agent may be used, such as one or more of those described in section IVB; in certain embodiments a combination of antivirals is used, such as a combination effective against HIV, where each antiviral may be attached to the same ligand, different ligands, or a combination thereof. In certain embodiments, the antimicrobial agent is an antifungal; any suitable antifungal agent may be used, such as one or more of those described in section IVC. In certain embodiments, the antimicrobial agent is an antiparasitic; any suitable antiparasitic agent may be used, such as one or more of those described in section IVD. The first (ligand targeting) moiety and the second (active agent, such as antimicrobial agent, e.g., antibiotic) may be linked by any suitable linkage. In certain cases, the two moieties are part of an overall structure, such as a NCE created from a known antibiotic with addition of a ligand targeting moiety. In certain cases, the two moieties are linked directly; in other cases they are linked indirectly via an intermediate moiety. The linkage may be covalent or noncovalent. Covalent linkages may be any suitable covalent linkage between functional groups on the moieties and/or functional groups on a linker; exemplary covalent linkages include an ester, carbonate, amide, imine, hydrazone or ether linkage. In certain embodiments, a covalent linkage between the two moieties is configured to be broken after the composition interacts with the cell that participates in infection healing. For example, the linkage may be stable to hydrolysis but cleaved by ROS; an exemplary linker of this type is acetal-boronate.

In certain embodiments, provided herein is a composition comprising an infection healing cell, such as an immune cell or a tissue repair cell, comprising an antimicrobial agent. The antimicrobial agent may be an antibiotic, antiviral, antifungal, or antiparasitic, such as described herein. In some cases, the antimicrobial is an antibiotic. In these embodiments, the antibiotic may be present in a concentration greater than that at which it would accumulate in the cell under normal physiological conditions, e.g., at normal concentrations of administration. This can be expressed as the ratio of intracellular concentration of the antibiotic to extracellular concentration of antibiotic, e.g., a ratio of at least 1.5, 2, 2.5, 3, 3.5, 4, 4.5, 5, 5.5, 6, 7, 8, 9, 10, 11, 12, 13, 14, 15, 16, 17, 18, 19, 20, 22, 25, 27, 30, 25, 40, 50, 60, 70, 80, 90, 100, 200, 300, 400, or 500 and/or not more than 2, 2.5, 3, 3.5, 4, 4.5, 5, 5.5, 6, 7, 8, 9, 10, 11, 12, 13, 14, 15, 16, 17, 18, 19, 20, 22, 25, 27, 30, 25, 40, 50, 60, 70, 80, 90, 100, 150, 200, 300, 400, 500, or 1000. Such a ratio may be determined by exposing a relevant infection healing cell, e.g., immune cell or tissue repair cell, to the antibiotic at a concentration equivalent to a concentration at which it would be present in the blood or tissue during normal administration, and determining the intracellular concentration after a suitable period of exposure to a composition comprising the antibiotic, or it may be determined from measurements of concentrations achieved intracellularly and extracellularly in vivo. The same applies to other antimicrobials, e.g., antifungals, antivirals, and antiparasites. In certain embodiments, the intracellular concentration of the antibiotic is at least 0.1 ng/1 and/or not more than 10 ug/ml. The antibiotic can be any antibiotic, e.g. an antibiotic as described in section IVA. In certain embodiments, the antibiotic is an antibiotic that does not normally accumulate intracellularly or does not substantially accumulate, such as a beta-lactam or a cephalosporin. The antimicrobial may associate with the cell intracellularly and/or extracellularly. In the case of intracellular accumulation, in some cases at least 50% of the antimicrobial is present in the cytosol. In certain cases, some or all of the antimicrobial is present in a cellular organelle, such as a lysosome, such as at least 10, 20, 30, 40, 50, 60, 70, 80, or 90%, of the antimicrobial. In certain embodiments, the immune cell and/or tissue repair cell comprising the antimicrobial is capable of normal or substantially normal function. The antimicrobial may be linked to a moiety that interacts with a target moiety of the immune cell and/or tissue repair cell, for active or passive transport. If the cell is an immune cell, the immune cell may be any immune cell as described herein, for example in section IIA; in certain embodiments, the immune cell is a phagocytic cell, for example, a neutrophil. A tissue repair cell can be any suitable cell, such as those described herein, e.g., a fibroblast, in some cases a differentiated fibroblast.

In certain embodiments, provided herein is a composition comprising (i) an infection healing cell, such as an immune cell or a tissue repair cell, comprising a membrane transporter for transporting a ligand across a cell membrane of the cell; and (ii) a ligand or derivative of a ligand, linked to an antimicrobial agent, wherein the ligand or ligand derivative is attached to the transporter, or is inside the cell. Suitable immune cells, transporters, ligands, and antimicrobials are as described elsewhere herein.

In certain embodiments, provided herein is a composition for treating a site of a drug-resistant bacterial infection comprising (i) an antibiotic specific for the drug-resistant bacteria linked to (ii) a ligand that targets infection healing cells, e.g., immune cells or tissue repair cells, at the site of infection or drawn to the site of infection. Suitable antibiotics, linkages, and ligands are as described elsewhere herein.

In certain embodiments, provided herein is a composition comprising a first antimicrobial agent that is preferentially accumulated by one or more types of target cells, such as infection healing cells, e.g., immune cells or tissue repair cells, linked to (ii) a second antimicrobial agent. In certain embodiments the first antimicrobial agent is an antibiotic, such as a macrolide, fluoroquinolone, a cephalosporin, or other antibiotic that is taken up by infection healing cells, such as immune cells or tissue repair cells. The second antimicrobial can be an antibiotic, antiviral, antifungal, or antiparasitic; in certain embodiments the second antimicrobial is an antibiotic. In certain embodiments both the first and the second antimicrobials are antibiotics, which may be the same antibiotic type or different, for example, a fluoroquinolone linked to a beta-lactam; flouroquinolones are known to accumulate in immune cells, such as neutrophils, whereas beta-lactams typically do not accumulate; thus, the beta-lactam is brought into the cell along with the fluoroquinolone. Another example is a macrolide, such as azithromycin linked to another antibiotic, e.g., fluoroquinolone; azithromycin is known to accumulate in immune cells (in lysosomes), in particular phagocytic cells such as neutrophils. The two antimicrobials may be directly or indirectly linked, as described herein. The linkage may be covalent or noncovalent. In certain embodiments, the linkage is configured to be cleaved when the composition reaches its site of use, e.g., inside an infection healing cell such as an immune cell or a tissue repair cell. In certain embodiments, the linkage is configured to not be cleaved when the composition reaches its site of use, e.g., inside an infection healing cell such as an immune cell or tissue repair cell, but one or both of the antibiotics remain active when linked.

In certain embodiments, provided herein is a composition comprising (i) a ligand that interacts with a moiety associated with an infection healing cell, e.g., an immune cell or a tissue repair cell, so as to increase the concentration of the ligand at the cell; (ii) a linker covalently linked to the ligand; and (iii) an antibiotic covalently linked to the ligand. The ligand can be any suitable ligand, for example as described herein; in certain embodiments the ligand is one that interacts with a transporter in a cell membrane, such as those described in section IIIB1. The antibiotic may be any suitable antibiotic, such as one of those described in section IVA; in certain embodiments, the antibiotic is a fluoroquinolone or a beta-lactam. In certain embodiments, the ligand is ascorbic or an ascorbic acid derivative, glucose or glucose derivative, DHA, mannose, galactose, amino acid, amino acid derivatives, carnitine, colistin, cephaloridine, ergothioneine, cytarabine, nucleotide, cytidine or derivatives, gemcitabine, cystine, cationic amino acids, cystathionine, glutamate. In certain embodiments, the ligand is linked to the antibiotic via an intermediate linker; in certain embodiments the intermediate linker is acetal-boronate.

In certain embodiments, provided herein is a pharmaceutical composition comprising an antimicrobial agent effective against one or more microbial agents linked to a ligand that interacts with a cell that participates in infection healing to concentrate the antimicrobial agent at the cell, and a pharmaceutically acceptable excipient. The cell may be any cell participating in infection healing (tissue repair), as described herein, such as an immune cell, e.g., a neutrophil, or a tissue repair cell, e.g., a fibroblast. The ligand may be any suitable ligand, such as one of those described in section III, such as ascorbic acid or a derivative thereof such as an aminoascorbic acid or dehydroascorbic acid or a derivative thereof. The antimicrobial agent may be any suitable antimicrobial agent, such as one of those described in section IV, such as an antibiotic, for example a fluoroquinolone or a beta-lactam. The pharmaceutically acceptable excipient may be any suitable excipient. As used herein, the term “pharmaceutically acceptable” includes a carrier that is compatible with the other ingredients of a pharmaceutical composition and can be safely administered to a subject. The term is used synonymously with “physiologically acceptable” and “pharmacologically acceptable”. Pharmaceutical compositions and techniques for their preparation and use are known to those of skill in the art in light of the present disclosure. For a detailed listing of suitable pharmacological compositions and techniques for their administration one may refer to texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008. Pharmaceutically acceptable carriers will generally be sterile, at least for human use. A pharmaceutical composition will generally comprise agents for buffering and preservation in storage, and can include buffers and carriers for appropriate delivery, depending on the route of administration. Examples of pharmaceutically acceptable carriers include, without limitation, normal (0.9%) saline, phosphate-buffered saline (PBS) Hank's balanced salt solution (HBSS) and multiple electrolyte solutions such as PlasmaLyte ATM (Baxter). Exemplary excipients include any that are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Pluronics, Triton-X, or polyethylene glycol (PEG). In certain embodiments, the pharmaceutical composition is suitable for oral administration. In certain embodiments, the pharmaceutical composition is suitable for administration by inhalation, e.g., a composition that can be aerosolized, such as a dry powder or an aqueous solution. In certain embodiments, the composition is suitable for cutaneous administration, e.g., systemic administration via cutaneous route, or topical administration. In certain embodiments, the composition is suitable for transdermal administration, e.g., to achieve systemic administration. In certain embodiments, the pharmaceutical composition is suitable for parenteral administration, e.g., intravenous, subcutaneous, intramuscular, or intrathecal injection. In certain embodiments, the pharmaceutical composition is suitable for intranasal administration. In certain embodiments, the pharmaceutical composition is suitable for rectal administration. In certain embodiments, the pharmaceutical composition is suitable for vaginal administration. In certain embodiments, the pharmaceutical composition is suitable for sublingual administration. In certain embodiments, the pharmaceutical composition is suitable for buccal administration. In certain embodiments, the pharmaceutical composition is suitable for ocular administration. In certain embodiments, the pharmaceutical composition is suitable for otic administration.

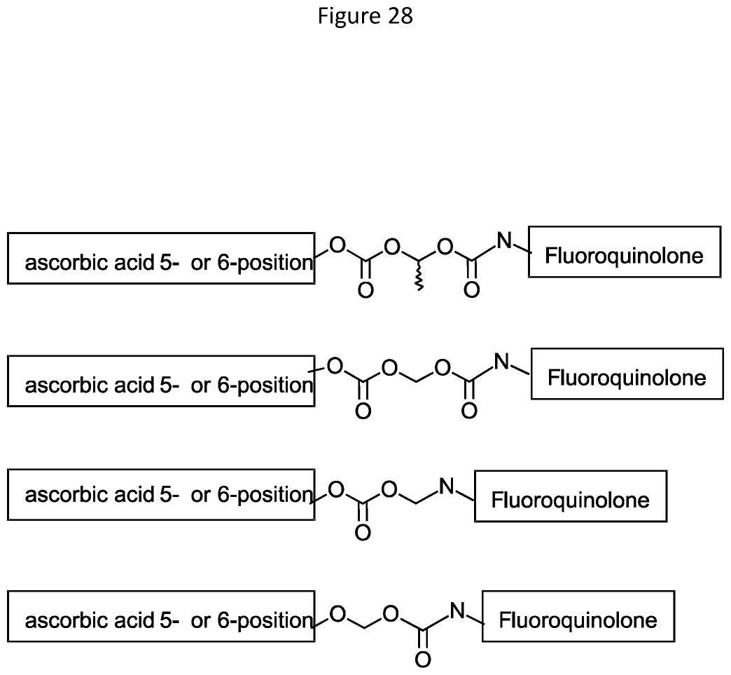

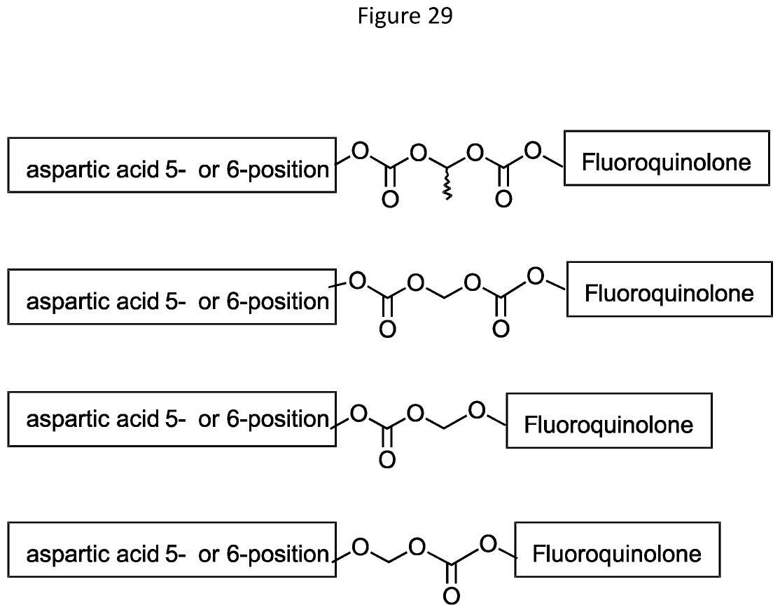

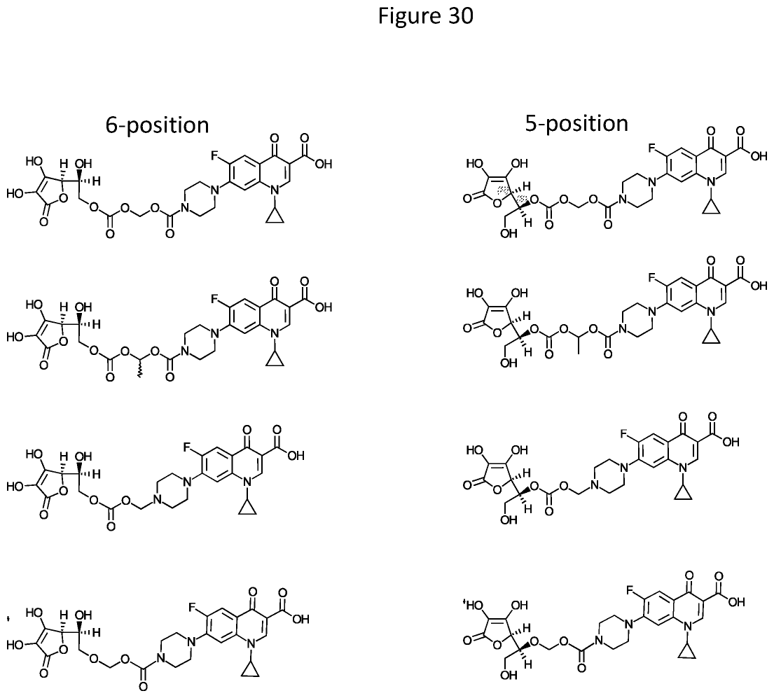

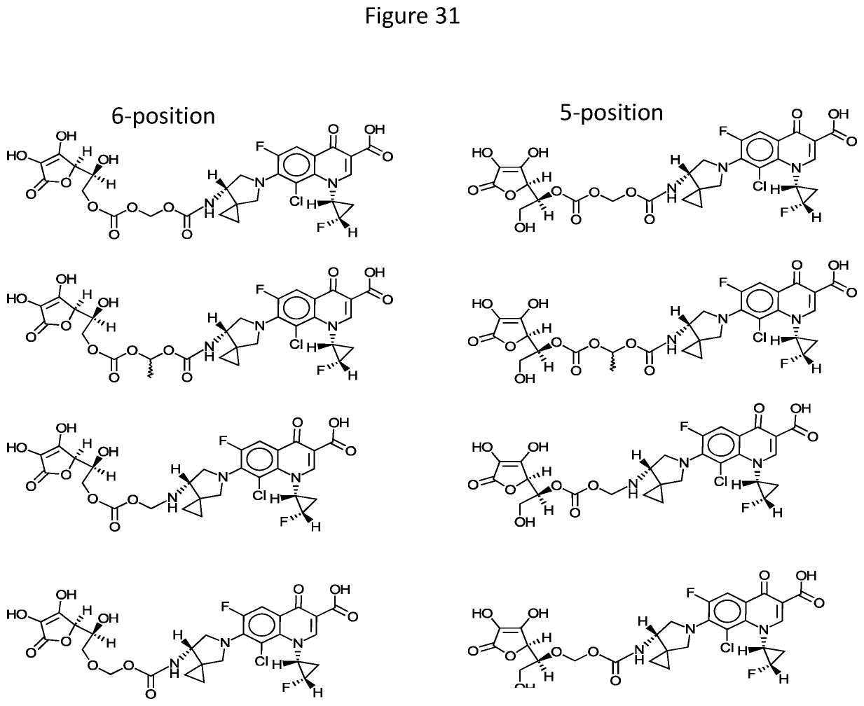

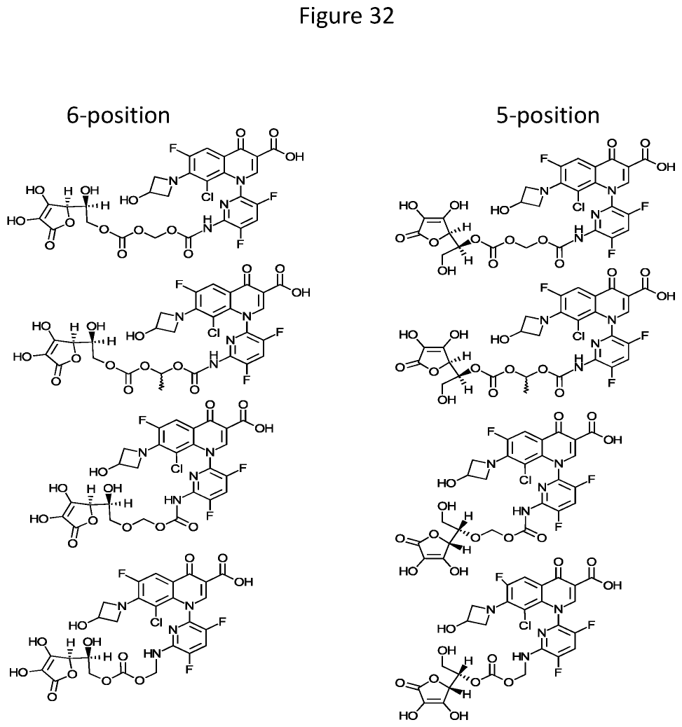

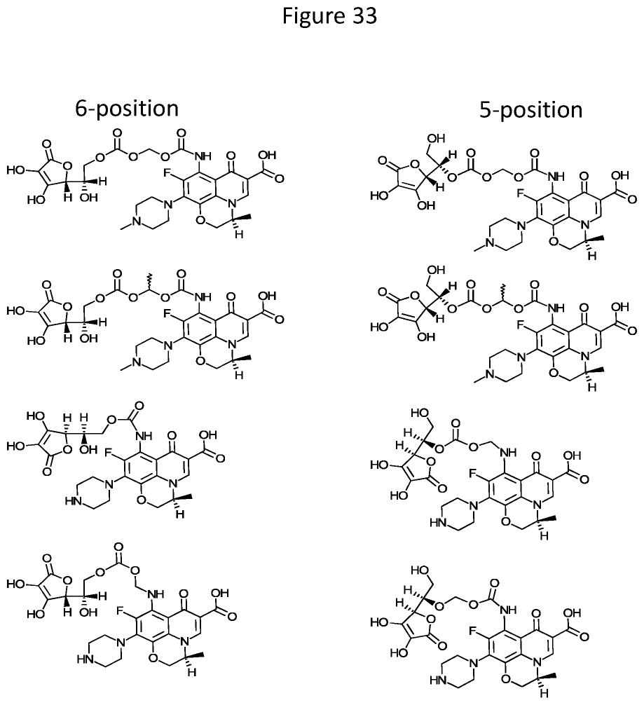

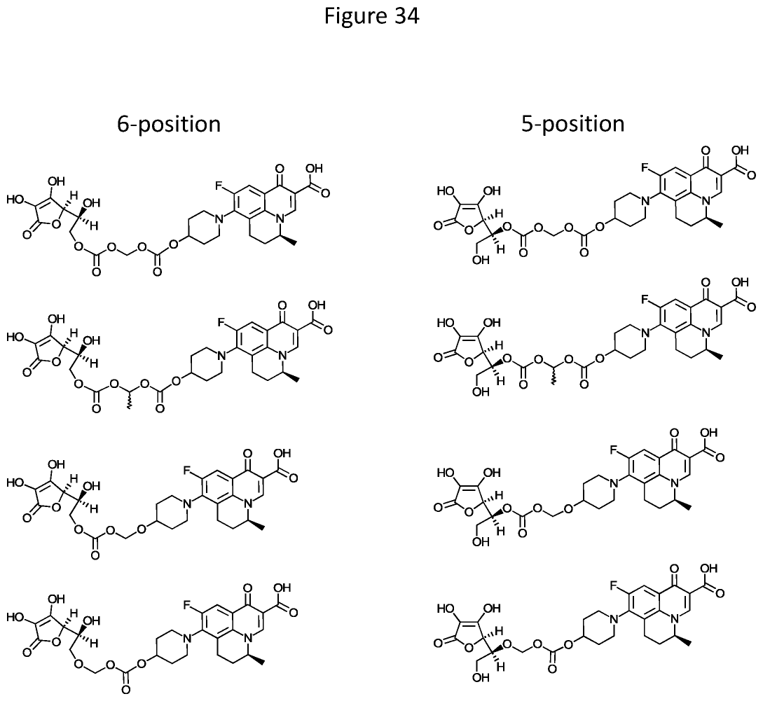

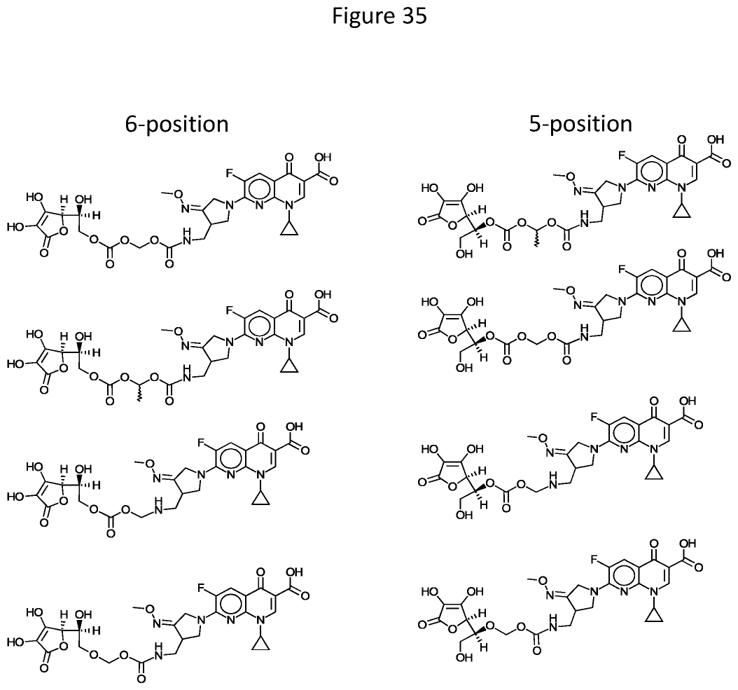

In certain embodiments, provided herein is a composition comprising (ii) ascorbic acid or an ascorbic acid derivative linked to (ii) an antimicrobial agent. In certain embodiments, the composition comprises ascorbic acid and the antimicrobial agent is covalently linked, directly or indirectly, to the ascorbic acid at the 5- or 6-hydroxyl position. In certain embodiments, the composition comprises 5-aminoascorbic acid and the antimicrobial agent is covalently linked, directly or indirectly, to the aminoascorbic acid at the 5-amino position or 6-hydroxyl position. In certain embodiments, the composition comprises 6-aminoascorbic acid and the antimicrobial agent is covalently linked, directly or indirectly, to the aminoascorbic acid at the 6-amino position or 5-hydroxyl position. Any suitable antimicrobial agent may be used, such as one of those described herein, e.g., in section IV. In certain embodiments, the antimicrobial agent comprises an antibiotic; any suitable antibiotic, such one of those described in section IVA, may be used. In certain embodiments, the antibiotic comprises a fluoroquinolone or a beta-lactam. In certain embodiments, the antibiotic comprises a fluoroquinolone, such as a fluoroquinolone of core structure A or B shown in FIG. 27, where two or more hydrogen atoms are replaced by carbon, oxygen, halogen, nitrogen or sulfur atoms. Primary and secondary amines aliphatic or aromatic or heteroaromatic as well as hydroxy groups constituting a part of active fluoroquinolone antibacterial can be employed as points of attachment of prodrug moieties. General structures of such prodrugs consisting of ascorbic acid moieties, linkers and fluoroquinolones are shown in FIG. 28 (amine group nitrogen linked fluoroquinolone structures) and FIG. 29 (hydroxy group oxygen linked fluoroquinolone structures. The linker shown is exemplary and not limiting. In certain embodiments, the composition comprises ascorbic acid linked through 6-position or 5-position to secondary aliphatic amine of a fluoroquinolone, for example, ciprofloxacin. See FIG. 30. The linker shown is exemplary and not limiting. In certain embodiments the composition comprises ascorbic acid linked through 6-position or 5-position to primary aliphatic amine of a fluoroquinolone as exemplified by sitafloxacin. See FIG. 31. The linker shown is exemplary and not limiting. In certain embodiments the composition comprises ascorbic acid linked through 6-position or 5-position to heteroaromatic amine of a fluoroquinolone as exemplified by delafloxacin. See FIG. 32. The linker shown is exemplary and not limiting. In certain embodiments the composition comprises ascorbic acid linked through 6-position or 5-position to an aromatic amine of a fluoroquinolone as exemplified by antofloxacin. See FIG. 33. The linker shown is exemplary and not limiting. In certain embodiments the composition comprises ascorbic acid linked through 6-position or 5-position to a hydroxy group of a fluoroquinolone as exemplified by levonadifloxacin. See FIG. 34. The linker shown is exemplary and not limiting. In certain embodiments the composition comprises ascorbic acid linked through 6-position and 5-position to a primary amino group of a fluoroquinolone Core B as exemplified by gemifloxacin. See FIG. 35. The linker shown is exemplary and not limiting.

In certain embodiments, provided herein is a composition comprising (i) a first antimicrobial agent that interacts with an infection healing cell in such a way as to increase the concentration of the antimicrobial agent at the infection healing cell, linked to (ii) a second antimicrobial agent. In certain embodiments, the first and second antimicrobial agents are different agents. In certain embodiments, the first and second antimicrobial agents are the same agent (i.e., two different moieties each of which has the same molecular structure). In certain embodiments, the infection healing cell comprises an immune cell; suitable immune cells are as described herein. In certain embodiments, the immune cell is a phagocyte, such as a neutrophil.

In certain embodiments, the infection healing cell comprises an wound repair cell; suitable wound repair cells are as described herein. In certain embodiments, the wound repair cell is a fibroblast. An exemplary combination includes a macrolide, such as azithromycin, linked to another antibiotic, e.g., a fluoroquinolone, where the macrolide acts as a targeting moiety; suitable macrolides and fluoroquinolones are as described herein. Another exemplary combination includes a fluoroquinolone linked to another antibiotic, e.g., a beta-lactam, where the fluoroquinolone acts as a targeting moiety; suitable fluoroquinolones and beta-lactams are as described herein.

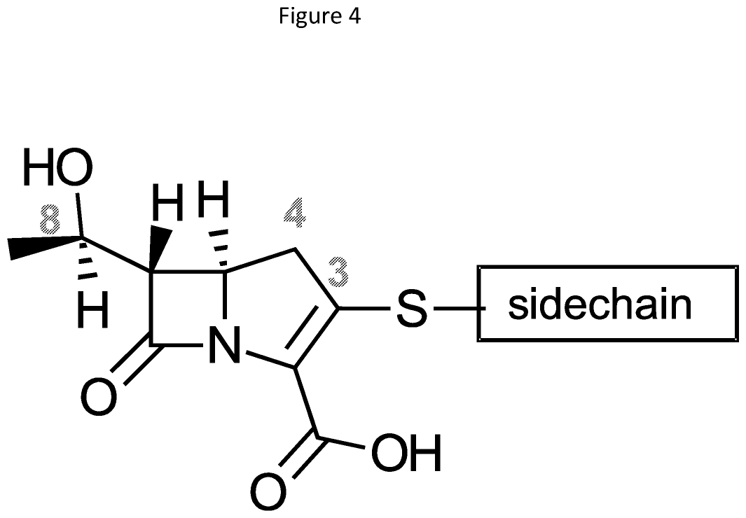

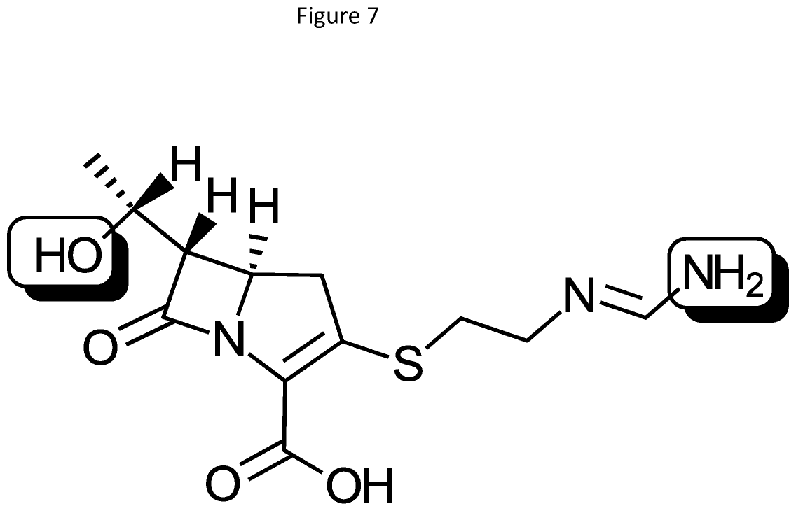

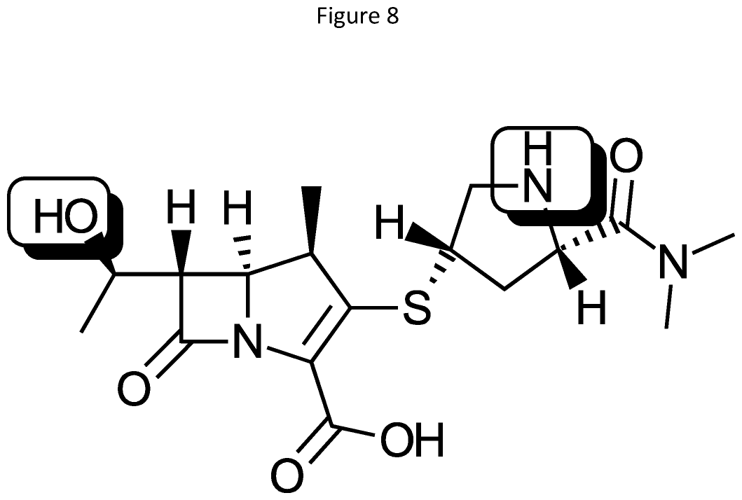

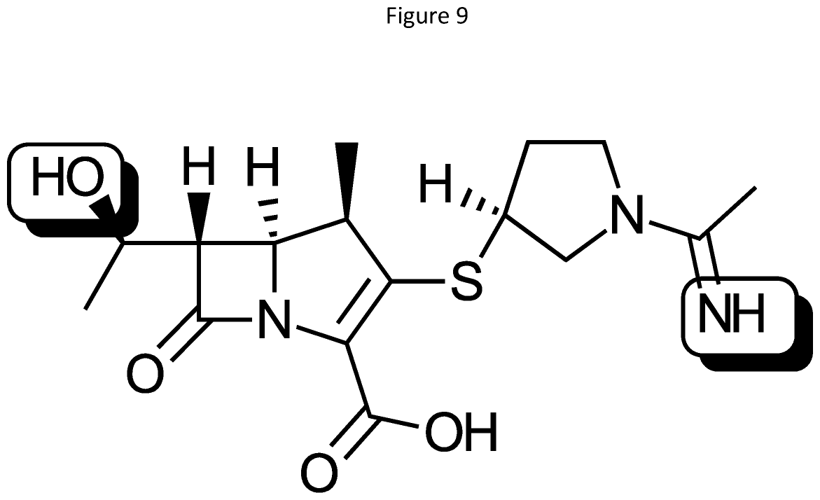

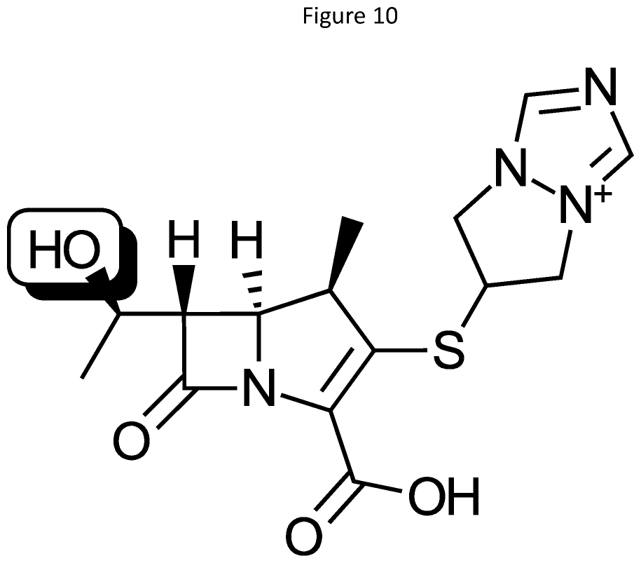

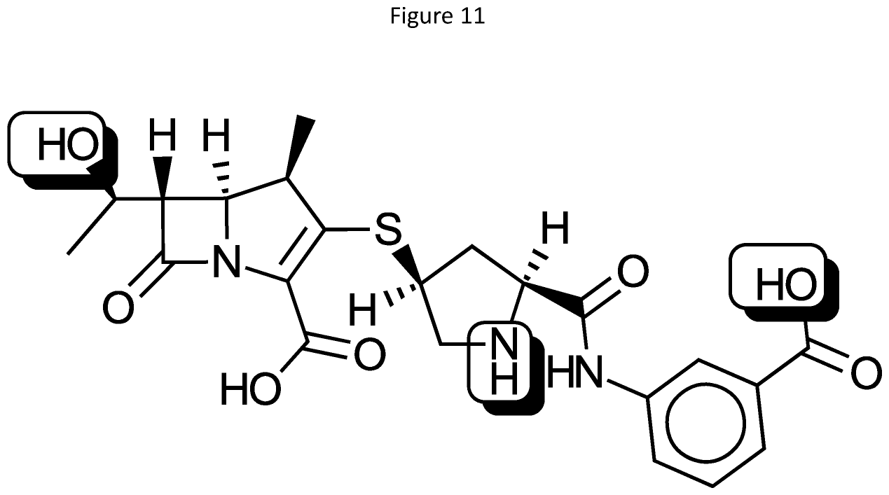

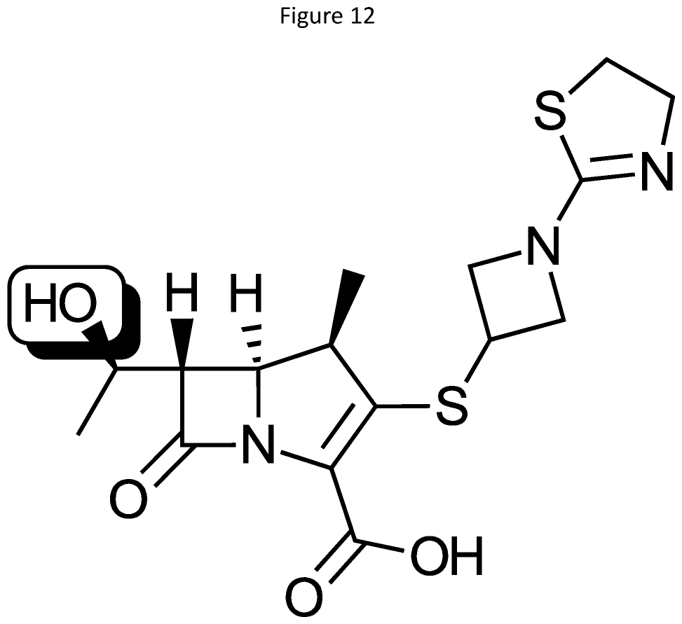

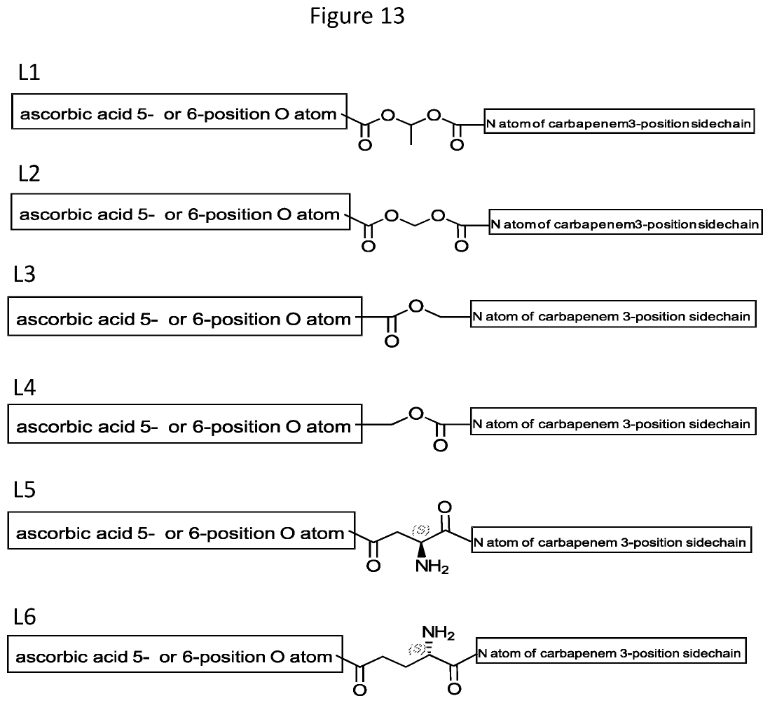

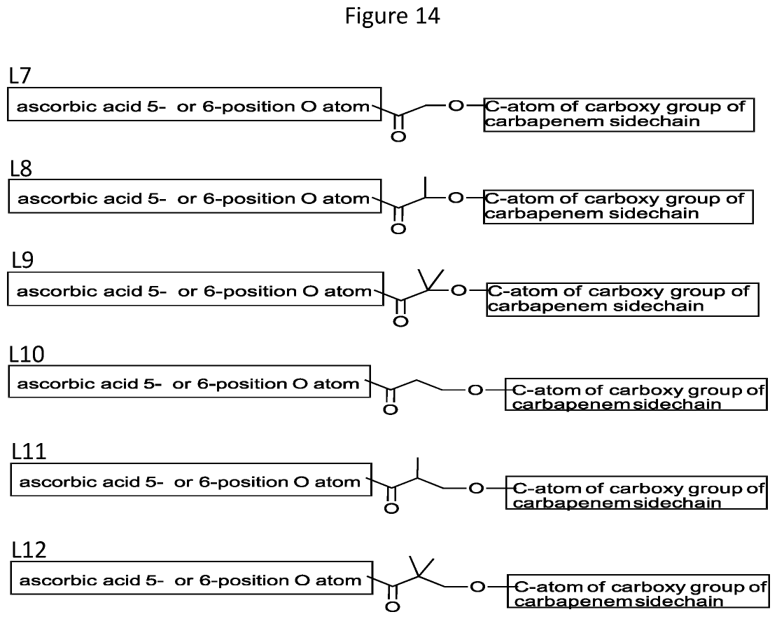

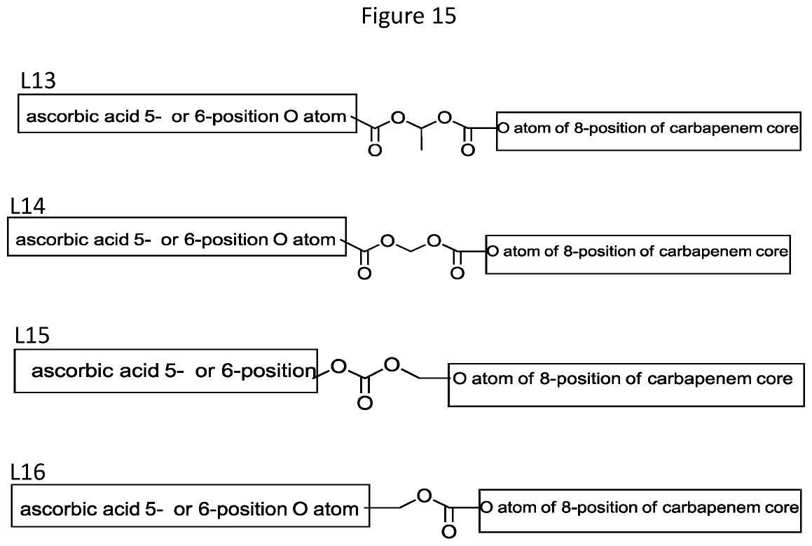

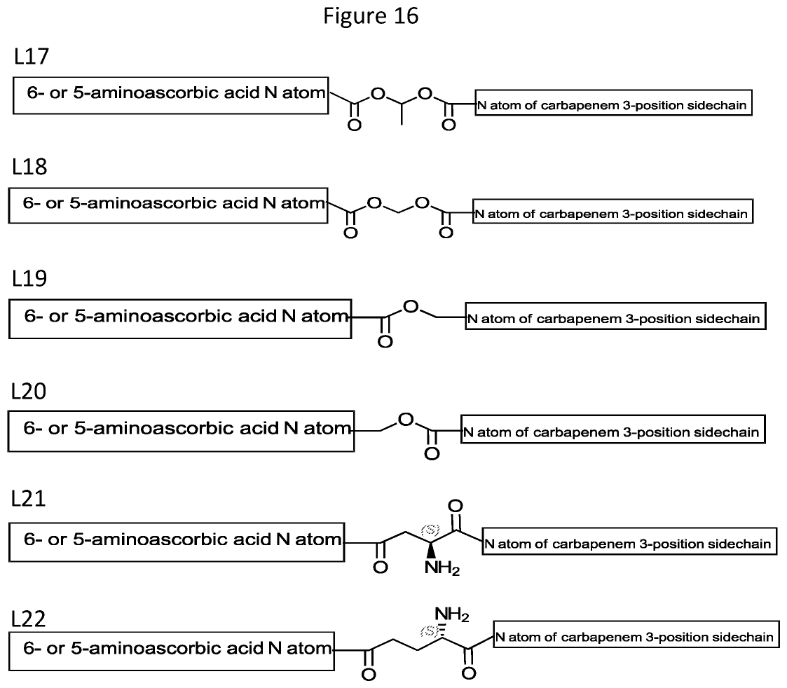

In certain embodiments, the composition comprises ascorbic acid, FIG. 1, or an aminoascorbic acid derivative, such as those shown in FIGS. 2 and 3, linked to a beta-lactam, such as a carbapenem, such as a carbapenem with a core structure shown in FIG. 4, 5, or 6. Exemplary carbapenems that may be linked, and potential sites for linkage, include imipenem (FIG. 7), meropenem (FIG. 8), panipenem (FIG. 9), biapenem (FIG. 10), ertapenem (FIG. 11), or tebipenem (FIG. 12). In general, functionalities other than acidic carboxy group of the carbapenem cores are used as point of attachments of the moiety leaving the carboxy group of carbapenem core unmodified in resulting structures. Amino or imino groups present at corresponding sidechains, hydroxy groups constituting a part of active carbapenem and carboxy groups which are not part of carbapenem core can be employed as points of attachment of prodrug moieties. General structures of such prodrugs consisting of ascorbic acid moieties, linkers and carbapenems are shown in FIGS. 13-17 (Note: in all general schemes bonds pointing toward boxes present open-ended bond to a particular nitrogen (N) or oxygen (O) atom. These bonds do not represent a carbon-carbon bond). Compositions of general types L1-L6 employing amino or imino groups of carbapenem 3-position sidechains linked with corresponding linkers to oxygen atom of 5- or 6-position of ascorbic acid are shown in FIG. 13. Compositions of general types L7-L12 employing carboxy groups of carbapenem 3-position sidechains linked with corresponding linkers to oxygen atom of 5- or 6-position of ascorbic acid are shown in FIG. 14. Compositions of general types L13-L16 employing carbapenem core 8-position oxygen linked with corresponding linkers to oxygen atom of 5- or 6-position of ascorbic acid are shown in FIG. 15. Compositions of general types L17-L22 employing carbapenem N atom of carbapenem 3-position sidechain linked with corresponding linkers to N atom of 5- or 6-position corresponding aminoascorbic acid are shown in FIG. 16.

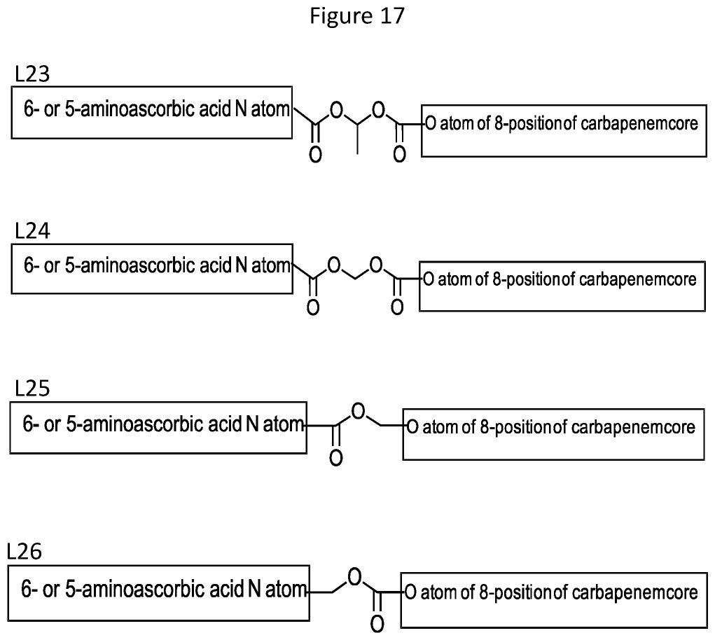

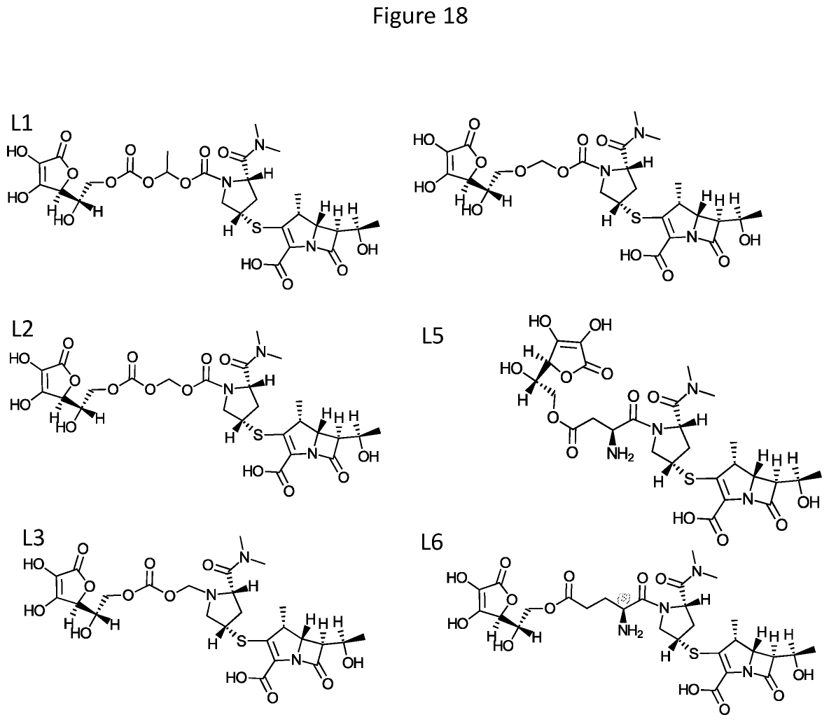

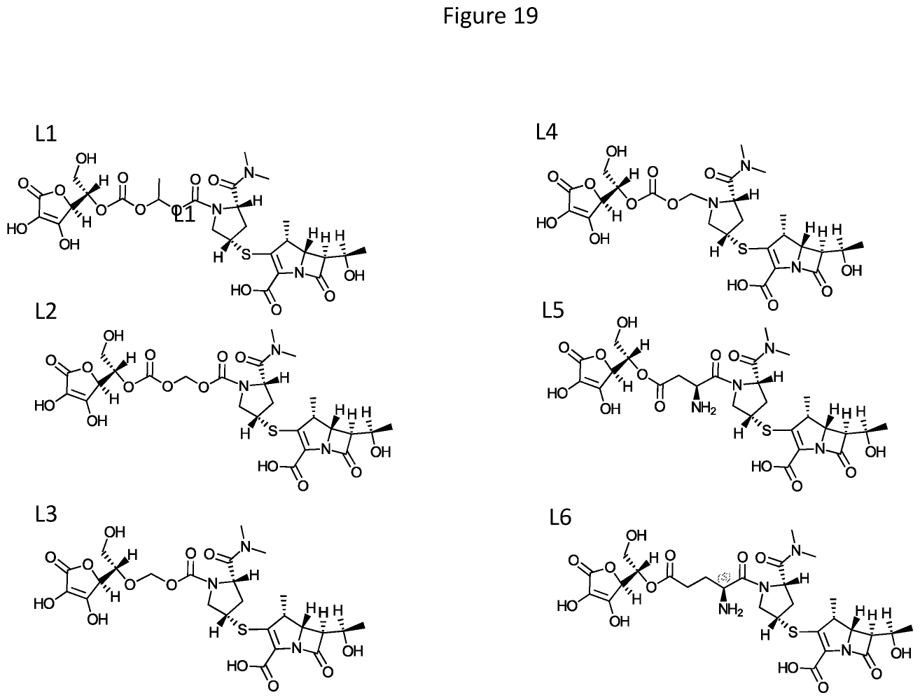

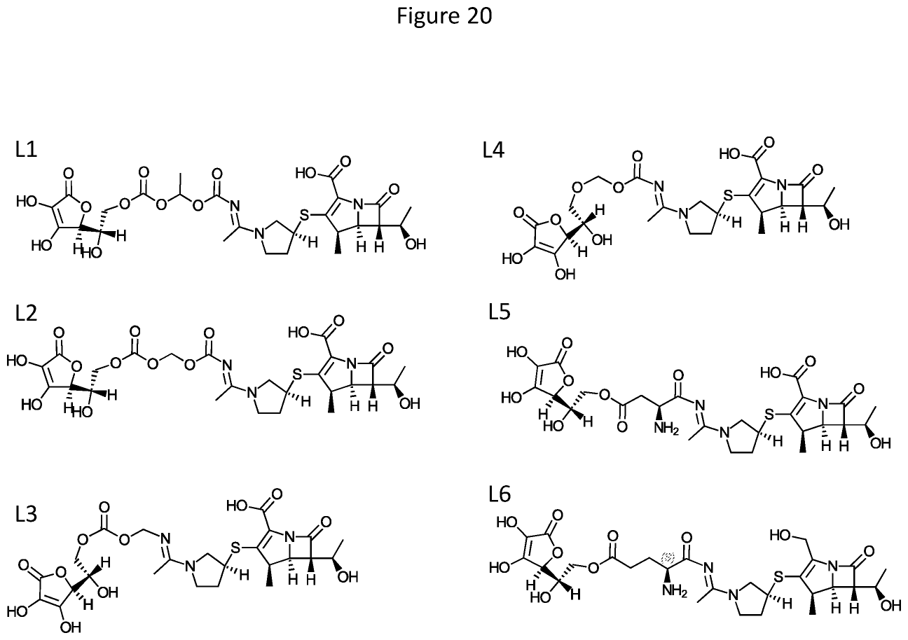

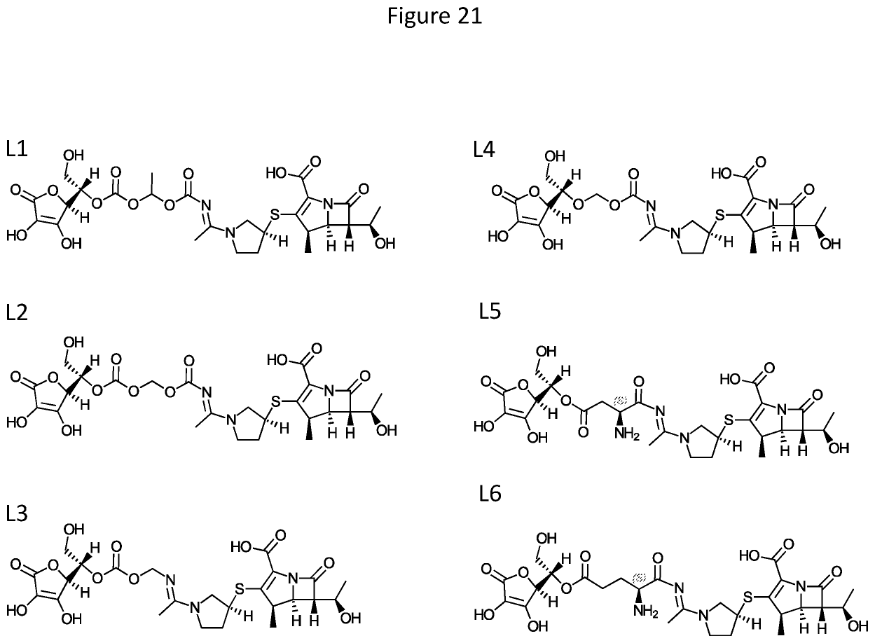

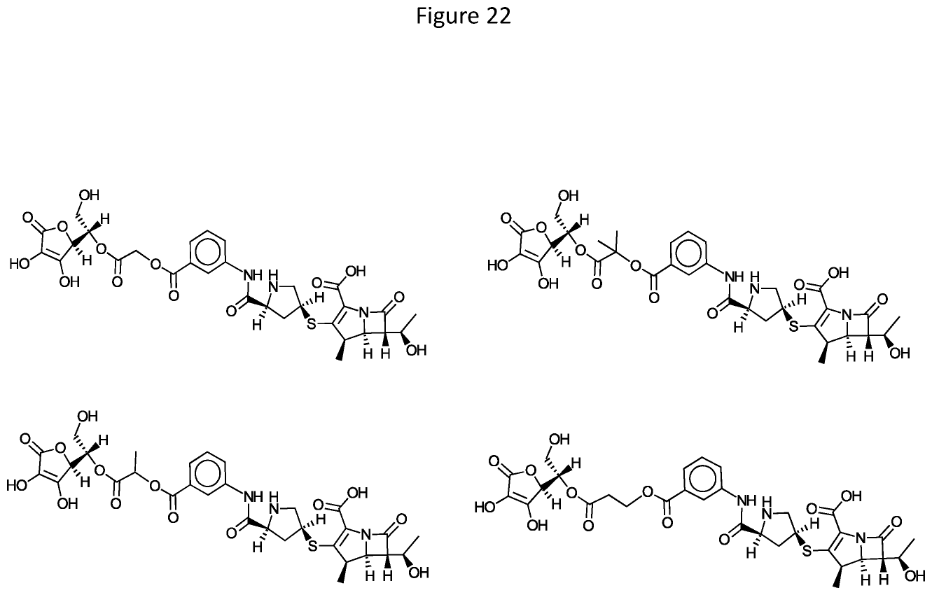

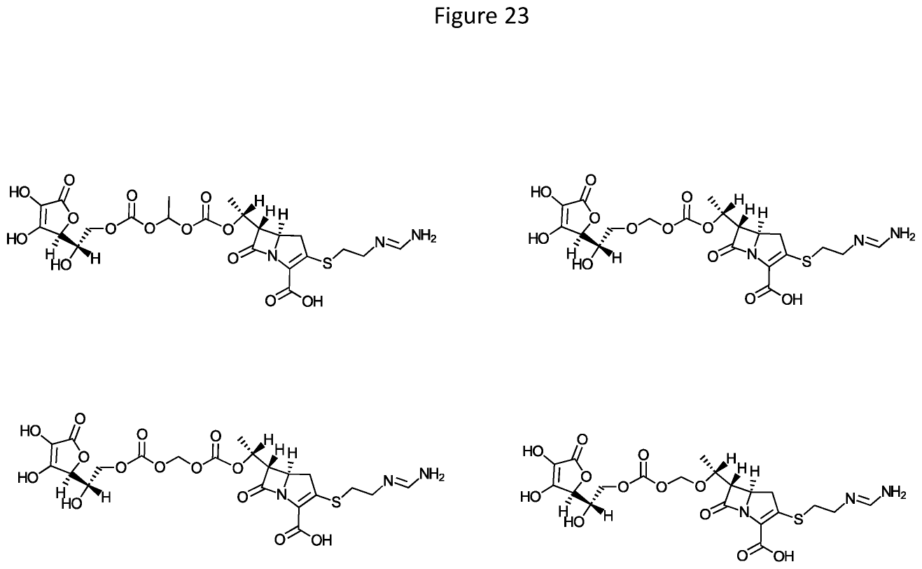

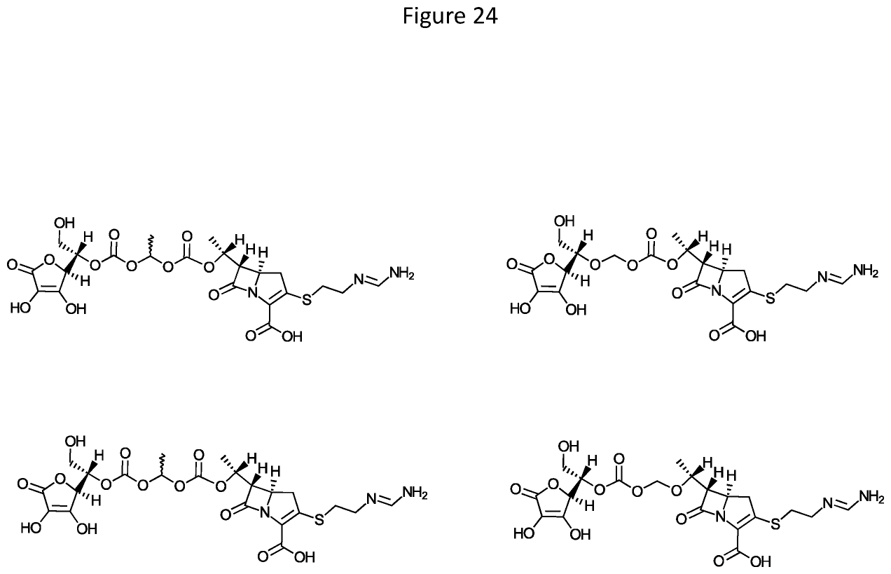

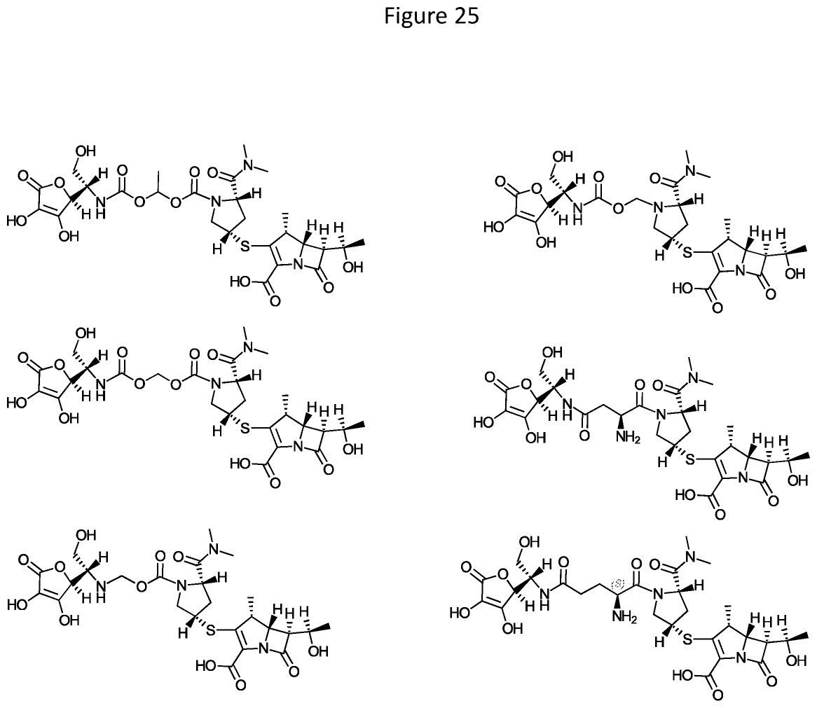

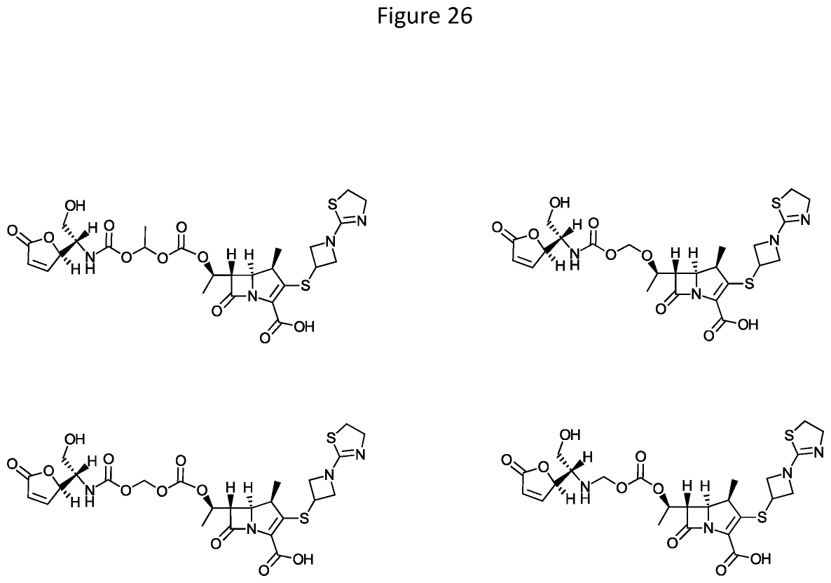

Compositions of general types L23-26 employing carbapenem core 8-position oxygen linked with corresponding linkers to N atom of 5- or 6-position corresponding aminoascorbic acid are shown in FIG. 17. FIGS. 18-26 show specific examples of various linkages; although the examples show specific carbapenems it is appreciated that any suitable carbapenem that is capable of forming the requisite linkages may be used. Compositions with ascorbic acid linked through 6-position to amine of a 3-position sidechain, as exemplified by meropenem (type L1-L6) are shown in FIG. 18. Compositions of type L1-L6 with ascorbic acid linked through 5-position to amine of a 3-position sidechain as exemplified by meropenem are shown in FIG. 19. In analogous fashion compositions of type L1-L6 with ascorbic acid linked through 6- or 5-position to an amine of a 3-position sidechain can be constructed of imipenem, doripenem and ertapenem. Compositions of type L1-L6 with ascorbic acid linked through 6-position to imine of a 3-position sidechain as exemplified by panipenem are shown in FIG. 20. Compositions of type L1-L6 with ascorbic acid linked through 5-position to imine of a 3-position sidechain as exemplified by panipenem are shown in FIG. 21. In analogous fashion compositions of type L1-L6 with ascorbic acid linked through 6- or 5-position to imine of a 3-position sidechain can be constructed of imipenem in its isomeric form of a terminal imine Compositions of general types L7-L12 employing carboxy groups of carbapenem 3-position sidechains linked with corresponding linkers to oxygen atom of 5-position of ascorbic acid as exemplified by ertapenem are shown in FIG. 22. In analogous fashion compositions of L7-L12 employing carboxy group of ertapenem 3-position sidechain linked with corresponding linkers to oxygen atom of 6-position of ascorbic acid can be constructed. Compositions of type L13-L16 with ascorbic acid linked through 6-position to oxygen atom at 8-position of carbapenem core as exemplified by imipenem are shown in FIG. 23. Compositions of type L13-L16 with ascorbic acid linked through 5-position to oxygen atom at 8-position of carbapenem core as exemplified by imipenem are shown in FIG. 24. In analogous fashion compositions of type L13-L16 with ascorbic acid linked through corresponding linkers to 6- or 5-position to oxygen atom at 8-position of carbapenem core can be constructed of imipenem, doripenem, biapenem, ertapenem and tebipenem. Compositions of type L17-L22 with 5-amino ascorbic acid linked through 5-position amine to amine of a 3-position sidechain as exemplified by meropenem are shown in FIG. 25. In analogous fashion compositions of type L17-L22 can be constructed with 6- or 5-position aminoascorbic acid linked through corresponding linkers to amine of 3-position sidechain of meropenem, imipenem, doripenem, ertapenem. Compositions of type L17-L22 with ascorbic acid linked through 6-position to oxygen atom at 8-position of carbapenem core as exemplified by tebipenem are shown in FIG. 26. In analogous fashion compositions of type L17-L22 can be constructed with 6- or 5-position aminoascorbic acid linked through corresponding linkers to oxygen atom at 8-position of carbapenem core of meropenem, imipenem, doripenem, biapenem, ertapenem and tebipenem.

In certain embodiments, provided herein is a composition comprising (i) a ligand targeting a target moiety associated with a natural killer (NK) cell or a T cell linked to (ii) a moiety comprising an antiviral agent. Ligands, links, and antiviral agents may be any suitable structures, for example those described in sections III, IVB, and V.

In certain embodiments, provided herein is a composition comprising (i) a ligand targeting a target moiety associated with a monocyte/macrophage linked to (ii) a moiety comprising an antifungal agent. Ligands, links, and antiviral agents may be any suitable structures, for example those described in sections III, IVC, and V.

In certain embodiments, provided herein is a composition comprising (i) a first moiety linked to (ii) a second moiety, wherein the first and second moieties are linked via a linker comprising acetal-boronate. The first moiety can be, e.g., a ligand, such as a ligand as described herein. The second moiety can be, e.g., an antimicrobial, such as an antibiotic, e.g., an antibiotic as described herein.

In certain embodiments, sometimes referred to herein as “prodrug” embodiments, a drug or drugs, such as an antibiotic, e.g., a commercially available antibiotic approved by the appropriate regulatory agencies, is linked to a ligand that is recognized by a suitable moiety of the target cells, such as a transporter (transporter-recognized ligand), and/or is passively transported into the target cell and captured (e.g. in lysosomes) where the linkage is configured to release the drug in a suitable environment, such as inside the cell, and/or in the surroundings of the cell; the released drug is in active form or is activated once released. In certain embodiments, sometimes referred to herein as “conjugate” embodiments, the drug is active or capable of activation while still attached to the ligand, and it is not necessary to release the drug from the ligand, i.e., the linkage between drug and ligand may remain intact once the drug is delivered to the target cell. In some of these embodiments, two or more drugs are linked, with or without separate transporter-recognized ligand attached, where at least one of the drugs is a drug that accumulates within the target cells even in the absence of ligand, so that one of the drugs acts not only to target the overall package but is, itself, an active agent. In certain embodiments, sometimes referred to herein as “NCE” embodiments, a new chemical entity is created with improved transport into target cells, e.g. immune cells such as white blood cells, e.g., lymphocytes or neutrophils, with or without a separate transporter-recognized ligand attached to it.

In certain embodiments, prodrugs of commercially available antibiotics are created that use transporters, whether active or passive, on infection healing cells such as immune cells or tissue repair cells, to carry an antibiotic to a site of infection. This can lead to a more effective treatment while lowering systemic exposure, and thus can eventually lead to reversal of antibiotic resistance. Although current approaches are especially aimed at developing antibiotics for S. pneumonia, Enterobacteriaceae, Neisseria gonorrhoeae, compositions and methods provided herein encompass more than these bacteria.

### VII. METHODS

In certain embodiments, provided herein is a method of accumulating an antimicrobial agent in an infection healing cell comprising (i) contacting the cell extracellularly with the antimicrobial agent linked to a ligand that accumulates in the infection healing cell; and (ii) allowing the antimicrobial agent linked to the ligand to be transported into the cell so that the antimicrobial agent accumulates in the cell. In certain embodiments the infection healing cell is an immune cell, e.g., phagocyte, such as a neutrophil, as described more fully elsewhere herein. In certain embodiments the infection healing cell is a wound repair cell, such as a fibroblast, also as described more fully elsewhere herein. In certain embodiments, the antimicrobial accumulates in an organelle of the cell, e.g., in lysosomes. In certain embodiments, the antimicrobial is an antibiotic, such as an antibiotic as described herein. In some cases, the linkage between the antimicrobial agent and the ligand is cleavable, and the method includes cleaving the linkage to release the antimicrobial agent. In some cases, the antimicrobial-linker bond can be broken releasing the antimicrobial while the linker-ligand bond remains intact. In some cases, the antimicrobial agent remains linked to the ligand; in such cases, generally the antimicrobial agent retains its normal activity or a substantial portion of its normal activity, such as at least 10, 20, 30, 40, 50, 60, 70, 80, 90, 95, 99, or 100% of normal activity. In some cases, the method further comprises releasing the antimicrobial agent into the extracellular environment, e.g., by lysing the cell or allowing the cell to be lysed, or other delivery method. In certain cases, the intracellular concentration of the antimicrobial agent in the cell increases so that it is at least 1.5, 2, 2.5, 3, 3.5, 4, 4.5, 5, 5.5, 6, 7, 8, 9, 10, 11, 12, 13, 14, 15, 16, 17, 18, 19, 20, 22, 25, 27, 30, 35, 40, 45, 50, 60, 70, 80, 90, or 100-fold the extracellular concentration of the antimicrobial agent, and/or no more than 2, 2.5, 3, 3.5, 4, 4.5, 5, 5.5, 6, 7, 8, 9, 10, 11, 12, 13, 14, 15, 16, 17, 18, 19, 20, 22, 25, 27, 30, 35, 40, 45, 50, 60, 70, 80, 90, 100, or 150-fold the extracellular concentration of the antimicrobial agent. In certain cases, the concentration of the antimicrobial agent in the cell is at least 1 pg, 10 pg, 100 pg, 1 ng, 10 ng, 100 ng, 1 ug, 10 ug, 100 ug, 1 mg, 10 mg, 100 mg and/or not more than 10 pg, 100 pg, 1 ng, 10 ng, 100 ng, 1 ug, 10 ug, 100 ug, 1 mg, 10 mg, 100 mg, or 1000 mg per milliliter.

In certain embodiments, provided herein is a method of accumulating an antimicrobial agent in a cell comprising (i) contacting the cell extracellularly with the antimicrobial agent linked to a ligand that interacts with a cell that participates in infection healing to concentrate the first ligand on or in the cell; (ii) allowing the antimicrobial agent linked to the ligand to accumulate in the cell. In some cases, the linkage between the antimicrobial agent and the ligand is cleavable, and the method includes cleaving the linkage to release the antimicrobial agent, either at the ligand-linkage site, the linkage-antimicrobial site, or both. In some cases, the antimicrobial-linker bond can be broken releasing the antimicrobial while the linker-ligand bond remains intact. In some cases, the antimicrobial agent remains linked to the ligand; in such cases, generally the antimicrobial agent retains its normal activity or a substantial portion of its normal activity, such as at least 10, 20, 30, 40, 50, 60, 70, 80, 90, 95, 99, or 100% of normal activity. In some cases, the method further comprises releasing the antimicrobial agent into the extracellular environment, e.g., by lysing the cell or allowing the cell to be lysed. In certain cases, the intracellular concentration of the antimicrobial agent in the cell increases so that it is at least 1.5, 2, 2.5, 3, 3.5, 4, 4.5, 5, 5.5, 6, 7, 8, 9, 10, 11, 12, 13, 14, 15, 16, 17, 18, 19, 20, 22, 25, 27, 30, 35, 40, 45, 50, 60, 70, 80, 90, or 100-fold the extracellular concentration of the antimicrobial agent, and/or no more than 2, 2.5, 3, 3.5, 4, 4.5, 5, 5.5, 6, 7, 8, 9, 10, 11, 12, 13, 14, 15, 16, 17, 18, 19, 20, 22, 25, 27, 30, 35, 40, 45, 50, 60, 70, 80, 90, 100, or 150-fold the extracellular concentration of the antimicrobial agent. In certain cases, the concentration of the antimicrobial agent in the cell is at least 1 pg, 10 pg, 100 pg, 1 ng, 10 ng, 100 ng, 1 ug, 10 ug, 100 ug, 1 mg, 10 mg, 100 mg and/or not more than 10 pg, 100 pg, 1 ng, 10 ng, 100 ng, 1 ug, 10 ug, 100 ug, 1 mg, 10 mg, 100 mg, or 1000 mg per milliliter.

In certain embodiments, provided herein is a method of delivering an antimicrobial agent to a site of an infection, mediated by one or more microbial agents, in an individual, comprising (i) administering to the individual a composition comprising an antimicrobial agent linked to a ligand that interacts with an infection healing cell, such as an immune cell or a tissue repair cell, to concentrate the antimicrobial agent at the infection healing cell, wherein the infection healing cell is a cell that is present at the site of infection or that preferentially travels to the site of infection; and (ii) allowing the antimicrobial agent to interact with the one or more microbial agents at the site of infection. In certain embodiments the infection healing cell is an immune cell, such as a phagocytic cell, e.g., a neutrophil. In certain embodiments the infection healing cell is a tissue repair cell, such as a fibroblast. In some cases, the concentration of the antimicrobial agent at the infection site increases so that the concentration is at least 1.5, 2, 2.5, 3, 3.5, 4, 4.5, 5, 5.5, 6, 7, 8, 9, 10, 11, 12, 13, 14, 15, 16, 17, 18, 19, 20, 22, 25, 27, 30, 35, 40, 45, 50, 60, 70, 80, 90, or 100-fold the concentration of the antimicrobial agent in the general circulation of the individual, and/or no more than 2, 2.5, 3, 3.5, 4, 4.5, 5, 5.5, 6, 7, 8, 9, 10, 11, 12, 13, 14, 15, 16, 17, 18, 19, 20, 22, 25, 27, 30, 35, 40, 45, 50, 60, 70, 80, 90, 100, or 150-fold the concentration of the antimicrobial agent in the general circulation of the individual. In certain cases, the method produces a concentration of the antimicrobial agent at the infection site is at least 1 pg, 10 pg, 100 pg, 1 ng, 10 ng, 100 ng, 1 ug, 10 ug, 100 ug and/or not more than 10 pg, 100 pg, 1 ng, 10 ng, 100 ng, 1 ug, 10 ug, 100 ug or 1000 ug, or 10 mg, 100 mg, or 1000 mg per milliliter. In certain embodiments at least one of the one or more microbial agents comprises an antibiotic-resistant bacterium.

In certain embodiments provided herein is a method of treating an infection caused by one or more microbial agents in an individual suffering from the infection comprising administering to the individual an effect amount of a composition comprising an antimicrobial agent effective against the one or more microbial agents linked to a ligand that interacts with an infection healing cell to concentrate the antimicrobial agent at the infection healing cell. In certain embodiments the infection healing cell is an immune cell, such as a phagocytic cell, e.g., a neutrophil. In certain embodiments the infection healing cell is a wound repair cell, such as a fibroblast. The infection may be a bacterial infection, a viral infection, a fungal infection, or a parasitic infection. In certain embodiments the infection is a bacterial infection and the antimicrobial agent is an antibiotic. The individual may be an animal; in certain embodiments, the individual is mammal, such as a human. Thus, individuals include mammals, such as humans and non-human primates, such as monkeys, as well as dogs, cats, horses, bovines, rabbits, rats, mice, goats, pigs, and other mammalian species. Subjects can also include avians. A patient can be an individual that is seeking treatment, monitoring, adjustment or modification of an existing therapeutic regimen, etc.

As used herein, the terms “effective amount,” “effective dose,” and “therapeutically effective amount,” include an amount of an agent, such as a composition as described herein, that is sufficient to generate a desired response, such as reduce or eliminate a sign or symptom of a condition or ameliorate a disorder. In some examples, an “effective amount” is one that treats (including prophylaxis) one or more symptoms and/or underlying causes of any of a disorder or disease and/or prevents progression of a disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control.

The terms “dose” and “dosage” are used interchangeably herein. A dose refers to the amount of active ingredient given to an individual at each administration. The dose will vary depending on a number of factors, including frequency of administration; size and tolerance of the individual; severity of the condition; risk of side effects; the route of administration. One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection. Dosage forms can be prepared for mucosal (e.g., nasal, sublingual, vaginal, buccal, or rectal), parenteral (e.g., subcutaneous, intravenous, intramuscular, or intraarterial injection, either bolus or infusion), oral, or transdermal administration to a patient. Examples of dosage forms include, but are not limited to: dispersions; suppositories; ointments; cataplasms (poultices); pastes; powders (e.g., powders for inhalation); dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient. In certain embodiments, the composition is given orally. The dose of composition to be administered is chosen in order to provide effective therapy for the patient and is in the range of less than 0.1 mg/kg body weight to about 25 mg/kg body weight or in the range 1 mg-2 g per patient. In some cases, the dose is in the range 1-100 mg/kg, or approximately 50 mg-8000 mg/patient. The dose may be repeated at an appropriate frequency which may be in the range once per day to once every three months, depending on the pharmacokinetics of the composition (e.g., half-life of the composition in the circulation) and the pharmacodynamic response (e.g., the duration of the therapeutic effect of the composition). In some embodiments, the in vivo half-life of between about 0.5 and about 25 days and composition dosing is repeated between once per every four hours and once every 3 months. Administration or use can be periodic. Depending on the route of administration, the dose can be administered, e.g., once every 0.25, 0.33, 0.5, 1, 2, 3, 4, 5, 6, 7, 10, 14, 21, or 28 days or longer (e.g., once every 2, 3, 4, or 6 months). In some cases, administration is more frequent, e.g., 2 or 3 times per day.

The patient can be monitored to adjust the dosage and frequency of administration depending on therapeutic progress and any adverse side effects, as will be recognized by one of skill in the art. Thus, in some embodiments, additional administration is dependent on patient progress, e.g., the patient is monitored between administrations. For example, after the first administration or round of administrations, the patient can be monitored for indications of infection, or general disease-related symptoms such as weakness, pain, nausea, etc. Often, a set course of administration is used regardless of clinical picture, except in the case of adverse effects. In therapeutic use for the treatment of an infection, a composition (e.g., including a therapeutic and/or diagnostic agent) can be administered at the initial dosage of about 0.001 mg/kg to about 1000 mg/kg daily and adjusted over time. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosage is varied depending upon the requirements of the patient, the severity of the condition being treated, and the targeted composition being employed. The dose administered to a patient, in the context of the present invention, should be sufficient to affect a beneficial therapeutic response in the patient over time. The size of the dose will also be determined by the existence, nature, and extent of any adverse side-effects that accompany the administration of a particular targeted composition in a particular patient, as will be recognized by the skilled practitioner.

The dosage of antimicrobial, e.g., antibiotic, contained in the composition to be administered is a suitable dosage; in some cases, the dosage is higher than the normal toxic dose for the antimicrobial, e.g., antibiotic, such as at least 1, 1.2, 1.5, 1.7, 2, 2.5, 3, 4, 5, 6, 7, 8, 9, or 10-fold the normal toxic dose. This can occur because, e.g. the antimicrobial, e.g., antibiotic, when bound in the composition, is not active or only partially active, and becomes completely active only when released at the site of the infection; thus, the systemic dose of active antibiotic is below toxic levels even the dosage in the composition as administered is above toxic levels. In certain embodiments, the concentration of active antimicrobial, e.g., antibiotic, achieved at the site of infection is at least 1.5, 2, 2.5, 3, 3.5, 4, 4.5, 5, 6, 7, 8, 9, 10, 12, 15, 20, 30, 40, 50, 70, or 100 times the concentration of active antibiotic in the blood and/or not more than 2, 2.5, 3, 3.5, 4, 4.5, 5, 6, 7, 8, 9, 10, 12, 15, 20, 30, 40, 50, 70, 100 or 200 times the concentration of active antimicrobial, e.g., antibiotic; in the blood.

In certain embodiments the infection is a bacterial infection. Any suitable bacterial infection may be treated using methods and compositions provided herein. Exemplary bacterial infections include gonorrhea, pneumonia, and food-borne diseases. In certain embodiments, the infection comprises an intracellular infection. In certain embodiments, the infection comprises an extracellular infection. In certain embodiments the bacterial infection is caused by one or more drug-resistant bacteria such as in Cystic-Fibrosis patients. One or more compositions as described herein that comprise at least one antibiotic moiety are administered to the individual at an effective dose, frequency, and duration. The composition may be administered after conventional antibiotic treatment has been tried, or may be administered without a conventional antibiotic treatment trial. The composition administered may be any suitable composition, such as the antibiotic compositions described herein. In some cases the composition administered comprises a fluoroquinolone antibiotic linked to a ligand that interacts with a target on one or more infection healing cells, e.g., immune cells or tissue repair cells, such as a macrolide antibiotic, e.g., azithromycin. In some cases the composition administered comprises a beta-lactam antibiotic linked to a ligand that interacts with a target on one or more infection healing cells, e.g., immune cells or tissue repair cells with little or no systemic toxicity, e.g., linked to a fluoroquinolone.

In certain embodiments, Gram-negative highly resistant bacteria are targeted, such as, Pseudomonas Aeruginosa, Acinetobacter baumannii, Enterobacteriaceae, Neisseria gonorrhoeae, Campylobacter spp., Salmonellae spp., Shigella spp. These can be targeted using any suitable antibiotic, e.g., using commercially available antibiotics with known mechanism of action (MOA). Targeted delivery of commercially available antibiotics results in higher efficacy, lower toxicity and an improved probability of success for development and registration. In certain embodiments, an antibiotic is used that is a broad spectrum antibiotic, such as fluoroquinolones and beta-lactams (e.g., cephalosporins, monobactams and carbapenem).

The mechanism of resistance for the fluoroquinolones and beta-lactams are known and well described in the literature. The approach of targeted delivery of commercially available antibiotics to the site of infection typically does not alter the MOA nor the mechanisms leading to resistance development. However, the approach reduces resistance because it increases the antibiotic concentration at the site of infection leading to a restoration of the susceptibility of the bacteria to the relevant antibiotic. This can apply to both intracellular and extracellular bacteria.

Thus, in certain embodiments, prodrugs of commercially available antibiotics with known safety and efficacy that target specific transporters on immune carrier cells to increase intracellular concentrations of antibiotics can be used. Antibiotic travels with the carrier cells and is present at the site of infection in active form; in some cases, the antibiotic is active even when linked to a target ligand, and in other cases the antibiotic is inactive or only partially active when linked to a target ligand and is released at the site of infection in its active form. This approach improves efficacy against both intracellular and extracellular pathogens. It can reduce systemic concentrations which may improve the overall safety profile of the therapeutic agent. Higher concentrations at the site of action eliminates highly resistant strains of bacteria and restore susceptibility of the bacteria to the antibiotic.

In certain embodiments, one of two classes of commercially available antibiotics that are considered the last resort to treat infections of highly resistant Gram-negative bacteria may be used. These include beta-lactams, in particular, carbapenem, and fluoroquinolones. In certain embodiments, the targeting moiety may be a macrolide antibiotic, e.g., in the case of a macrolide-fluoroquinolone composition.

In certain embodiments, provided herein is a method of transporting an antimicrobial agent into a cell comprising contacting the cell with an effective amount of a composition comprising a ligand for a transporter in the plasma membrane of the cell linked to the antimicrobial agent under conditions wherein the ligand binds to the transporter and is carried into the cell along with the antimicrobial agent.

Thus, in certain embodiments provided herein is a novel approach to combat antibacterial resistance utilizing endogenous body infection healing cells, such as immune cells and/or tissue repair cells (carrier cells) for targeted delivery of the antibiotics to the site of infection. Developing oral antibiotics for resistant bacteria, especially for Gram-negative infections, is globally recognized as an urgent unmet medical need. However, the technology can be applied to other infectious diseases including anti-viral and anti-fungal therapies. Targeted delivery combining the power of infection healing cells, e.g., immune cells and/or tissue repair cells, armed with commercially available antibiotics to deliver higher concentrations at the site of action can result in: improved efficacy for both intracellular and extracellular bacteria; improved efficacy in low perfusion tissues due to improved distribution and increased half-life; improved efficacy in certain diseases such as cystic fibrosis and diabetes; reversal of resistance by restoring the susceptibility of the bacteria; improved safety profile by reducing systemic exposure; increased likelihood of success as entities with known efficacy and safety are being used; shorter clinical development times; fast track status (QIDP designation); and/or additional 5-year of exclusivity (GAIN act)

An important issue is to select the combination of infection healing cells, e.g., immune and/or tissue repair cells, transporters and antibiotics. One approach is to follow both the CDC and WHO recommendations related the most unmet medical need. A candidate can meet the following criteria: prodrug has sufficient stability in plasma; prodrug accumulates at desired infection healing cells, e.g., immune cells or tissue healing cells; prodrug does not interfere or substantially interfere with infection healing cell, e.g., immune cell or tissue repair cell migration; antibiotic is active (e.g., released) at the site of infection.

A battery of in vitro and in situ studies can be identified and/or developed. These screening studies ensure that the prodrug meets certain criteria. For example, stability studies in blood/plasma can be used to screen stability. Accumulation in the infection healing cells, e.g., immune cells or tissue repair cells, can be achieved using in situ assay in, e.g., freshly isolated cells. In vivo PK/PD studies in animals can be used to confirm the hypothesis that the antibiotic is accumulating in the cells and is active, e.g., released to site of infection, and comparative studies in animal model of infectious disease can identify a safe and effective dose. Clinical Phase 1 PK/PD study can be utilized to confirm hypothesis in human and Phase 3 trial(s) can be used to provide the ultimate evidence of safety and efficacy in hard to treat infections.

Thus, in certain embodiments, provided herein are methods and compositions for selecting suitable targeted drug compositions. For example, a large library of novel prodrugs that utilizes the infection healing cells, e.g., immune cells or tissue repair cells, as a carrier in combination with multiple classes of suitable antibiotics, e.g., marketed antibiotics, may be synthesized. Once synthetized these prodrugs are screened, based on preset criteria, and the lead candidate(s) can be selected for ADMET (absorption, distribution, metabolism, elimination, and toxicity) and in vivo animal studies.

In certain embodiments, if commercially used antibiotics are used, they can selected for their broad-spectrum activity, known potency against hard to treat infections, e.g., caused primarily by Gram-negative bacteria with proven wide therapeutic range. Ciprofloxacin and meropenem have been used as a last resort for hard to treat bacteria. However, the incidence of resistance against these antibiotics is rising and threatens to limit the gains that have been made. The approach described herein combines the power of immune system and the commercially available antibiotics to increase potency by elevating the concentrations of the antibiotics at the site of action.

Provided herein are methods for treating an infection, using one or more of the agents described herein; such methods include delivering the agent to an individual suffering from an infection. Any suitable method of delivering an agent as provided herein may be used. In certain embodiments, intravenous (IV) and/or oral delivery is used. The IV can be used in ICU setting while the oral allows treatment continuation at home. This is merely exemplary and any suitable mode of delivery or combination of modes may be used, for example, inhalation for, e.g., treatment of pulmonary conditions.

Thus, in certain embodiments provided herein is the synthesis of prodrugs using commercially available antibiotics. These prodrugs target specific transporters of endogenous immune carrier cells to increase intracellular concentrations of antibiotics. As the infection healing cells, e.g., immune cells or tissue repair cells, are attracted to the site of infection, the antibiotics travel with them and are active, e.g., released at the site of infection in their active form. This approach improves efficacy against both intracellular and extracellular pathogens. Due to the targeted delivery the prodrug is removed from the blood stream resulting in lower drug exposure in the systemic circulation which in turn will improve upon side-effect profile of the therapeutic agent. Use of existing drugs with proven safety and efficacy shortens the development phase and increase the probability of success.

### Treatment of Pulmonary Bacterial Infection

In certain embodiments, provided herein is a method of treating a subject, such as a mammal, e.g., a human subject, suffering from a pulmonary bacterial infection by administering a therapeutically effective amount of an aerosolized composition, e.g., a liquid formulation such as an aqueous formulation, a dry powder formulation, or a liposomal formulation.

Administration is by inhalation, and any form of the composition that is suitable for administration by inhalation may be used. In certain embodiments, administration can be systemic, e.g., as a supplement to administration by inhalation; in such cases, the appropriate dosage form of the composition is used. The composition can be, e.g., a composition comprising (i) a first moiety comprising a ligand that interacts with a cell that participates in infection healing to concentrate the first moiety on or in the cell, linked to (ii) a second moiety comprising an antimicrobial agent, such as an antibiotic. In certain embodiments the subject is a human with pneumonia, a chronic obstructive pulmonary disease, chronic bronchitis, bronchiectasis, asthma, sinusitis, rhinosinusitis, orcystic fibrosis, or a human being mechanically ventilated. In certain embodiments the subject is a human with pneumonia. In certain embodiments the subject is a human with COPD. In certain embodiments the subject is a human with chronic bronchitis. In certain embodiments the subject is a human with bronchiectasis. In certain embodiments the subject is a human with asthma. In certain embodiments the subject is a human with sinusitis. In certain embodiments the subject is a human with rhinosinusitis. In certain embodiments the subject is a human with cystic fibrosis. In certain embodiments the subject is a human that is mechanically ventilated.

The infection can be any pulmonary infection suitable for treatment with aerosolized form of a composition as described herein. In certain embodiments the infection comprises one or more bacteria that can include Pseudomonas aeruginosa, Pseudomonas fluorescens, Pseudomonas acidovorans, Pseudomonas alcaligenes, Pseudomonas putida, Stenotrophomonas sp., e.g., Stenotrophomonas maltophilia, Aeromonas hydrophilia, Escherichia coli, Citrobacter freundii, Salmonella typhimurium, Salmonella typhi, Salmonella paratyphi, Salmonella enteritidis. Shigella dysenteriae. Shigella flexneri, Shigella sonnei, Enterobacter cloacae, Enterobacter aerogenes, Klebsiella pneumoniae, Klebsiella oxytoca, Serratia marcescens, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi. Pasteurella multocida, Pasteurella haemolytica, Helicobacter pylori, Campylobacter fetus, Campylobacter jejuni, Campylobacter coli, Borrelia burgdorferi, Vibrio cholera, Vibrio parahaemolyticus, Legionella pneumophila, Listeria monocytogenes, Neisseria gonorrhoeae, Neisseria meningitidis, Burkholderia sp., e.g., Burkholderia cepacia, Francisella tularensis, Kingella, Moraxella, or a combination of two or more of the above.

In certain embodiments, the pulmonary infection can include a gram-negative anaerobic bacteria. In certain embodiments, the pulmonary infection can include one or more of Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, and Bacteroides splanchnicus.

In certain embodiments, the pulmonary infection can include a gram-positive bacteria. In certain embodiments, the pulmonary infection can include one or more of Corynebacterium diphtheriae, Corynebacterium ulcerans, Streptococcus pneumoniae, Streptococcus agalactiae, Streptococcus pyogenes, Streptococcus milleri; Streptococcus (Group G); Streptococcus (Group C/F); Enterococcus faecalis, Enterococcus faecium, Staphylococcus aureus, Staphylococcus epidermidis, Staphylococcus saprophyticus, Staphylococcus intermedius. Staphylococcus hyicus subsp. hyicus, Staphylococcus haemolyticus, Staphylococcus hominis, and Staphylococcus saccharolyticus.

In some embodiments, the pulmonary infection can include a gram-positive anaerobic bacteria. In some embodiments, the pulmonary infection can include one or more of Clostridium difficile, Clostridium perfringens, Clostridium tetini, and Clostridium botulinum.

In certain embodiments, the pulmonary infection can include an acid-fast bacteria. In certain embodiments, the pulmonary infection can include one or more of Mycobacterium tuberculosis, Mycobacterium avium, Mycobacterium intracellulare, and Mycobacterium leprae.

In certain embodiments, the pulmonary infection can include an atypical bacteria. In certain embodiments, the pulmonary infection can include one or more of Chlamydia pneumoniae and Mycoplasma pneumoniae.

In certain embodiments, the pulmonary infection can comprise a non-fermenting gram-negative bacteria (NFGNB). Examples of NFGNB can include Burkholeria spp., Stenotrophomonas spp., Acinetobacter spp., Pseudomonas spp., and Achromobacter spp.

In certain embodiments, the bacterial infection is an antibiotic-resistant bacterial infection. In certain embodiments, the bacterial infection comprises Pseudomonas bacteria, such as is Pseudomonas aeruginosa, Pseudomonas fluorescens, Pseudomonas acidovorans, Pseudomonas alcaligenes, Pseudomonas putida, or a combination of two or more thereof. In certain embodiments, the infection is a Pseudomonas aeruginosa infection. In certain embodiments, the bacterial infection is a methicillin resistant Staphylococcus aureus (MRSA) infection. In certain embodiments, the infection is a Streptococcus pneumonia (Sp) infection. In certain embodiments, the infection comprises one or more Mycobacterium, such as one or more of Mycobacterium tuberculosis, Mycobacterium avium, Mycobacterium intracellulare, or Mycobacterium leprae, for example Mycobacterium avium or Mycobacterium intracellulare. In certain embodiments, the bacterial infection comprises Haemophilus influenzae. In certain embodiments the bacterial infection comprises Haemophilus parainfluenza. In certain embodiments the bacterial infection comprises Moraxella catarrhalis.

In certain embodiments, the composition is an aqueous composition. In certain embodiments, the composition is a dry powder formulation. In certain embodiments, the composition is a liposomal composition. In certain embodiments, the composition comprises a combination of formulations, e.g., aqueous solution and liposomal suspension; such a formulation can allow for both immediate effects, e.g., from the aqueous solution, and longer-term effects, e.g., from liposomes. Distribution of the different formulations may also be different, increasing effectiveness. The antimicrobial, e.g., antibiotic, in the aerosolized composition may be in any suitable form.

In certain embodiments, the composition is administered with a divalent or trivalent cation, or combination thereof, such as magnesium, calcium, zinc, copper, aluminum, or iron, or a combination thereof; in certain embodiments, the composition is administered with a divalent cation, such as magnesium or calcium; in certain embodiments, the composition is administered with a divalent cation, such as magnesium, for example, magnesium chloride. In liquid formulations, e.g., aqueous formulations, concentrations of the divalent or trivalent cation, or combination thereof, e.g., magnesium, such as magnesium chloride, may be any suitable concentration, such as 50-400 mM, e.g., where the concentration of the antimicrobial, e.g., antibiotic, is 5-80, 10-70, 20-60, 20-50, 20-40, 30-100, 40-100, 50-120, 60-120, or 50-200 mg/ml, or 100-300 mM, e.g., where the concentration of the antimicrobial, e.g., antibiotic, is 75-150 mg/ml, or 150-250 mM, or, e.g., where the concentration of the antimicrobial, e.g., antibiotic, is 5-80, 10-70, 20-60, 20-50, 20-40, 30-100, 40-100, 50-120, 60-120, or 90-125 mg/ml.

In certain embodiments, the composition is an aqueous composition. In these embodiments, the osmolarity of the composition may be any suitable osmolarity, for example 200-1250, 250-1050, 300-500, 350-750, or 350-425 mOsmol/kg. A permeant ion concentration may be any suitable concentration as described herein, for example 30-300 mM, such as 50-200 mM. In one such embodiment, one or more permeant ions in the composition are selected from the group consisting of chloride and bromide. In certain embodiments, the composition comprises a taste-masking agent, which can be any suitable taste-masking agent, such as a sugar, a divalent or trivalent cation or combination thereof that associates with the composition, optimized osmolality, and/or an optimized permeant ion concentration. pH can be any suitable pH, e.g., 5-8, 5-7.5, 5-7, 5-6.5, 5-6, 5.5-8, 5.5-7.5, 5.5-7, 5.5-6.5, 6-8, 6-7.5, 6-7, 6-6.5, 6.5-8, 6.5-7.5, or 6.5-7. In certain embodiments the pH is 5-8. In certain embodiments the pH is 5-6.5. In certain embodiments the pH is 5.5-6.5.

In certain embodiments the composition is an aqueous composition with a divalent cation, e.g., magnesium, at a concentration of 50-400 mM, a pH of 5-8, and an osmolarity of 200-1250 mOsmol/kg.

In certain embodiments, the composition is an aqueous composition comprising an antimicrobial, e.g., antibiotic, at a concentration between 5-80, 10-70, 20-60, 20-50, 20-40, 30-100, 40-100, 50-120, 60-120, or 50-200 mg/ml, such as 20-100 mg/ml, or 20-80 mg/ml, or 30-100 mg/ml, or 30-80 mg/ml, or 80-150 mg/ml, in some cases 90-110 mg/ml, a magnesium chloride concentration of 100-400 mM, such as 125-300 mM, in some cases 175 mM to about 225 mM, and a pH of 5-8, in some cases 5-7.5, such as 5-7; an osmolarity of 200-1250 mOsmol/kg, in some cases 250-1050 mOsmol/kg, for example 250-550 mOsmol/kg, in particular 300-500 mOsmol/kg, and, optionally, lacks lactose. In certain embodiments the composition is an aqueous composition with a concentration of antimicrobial, e.g., antibiotic, of 20-50 mg/ml or 90-110 mg/ml, a magnesium chloride concentration of 175-225 mM, a pH of 5-7; an osmolarity of 300-mOsmol/kg. In certain embodiments the composition lacks lactose.

In certain embodiments, the composition is a dry powder composition, such as any suitable dry powder composition, e.g. a dry powder composition with or without a blending agent such as lactose.

In certain embodiments, the composition is a liposomal composition, such as any suitable liposomal composition.

The composition is administered in any suitable manner, depending on the nature of the composition, e.g., by liquid nebulizer, dry powder inhaler, ventilator, or any other suitable method.

In certain embodiments the duration of a therapy, e.g., with an aerosolized composition comprising (i) a first moiety comprising a ligand that interacts with a cell that participates in infection healing to concentrate the first moiety on or in the cell, linked to (ii) a second moiety comprising an antimicrobial agent, can include at least about 1 day/month, at least about 2 days/month, at least about 3 days/month, at least about 4 days/month, at least about 5 days/month, at least about 6 days/month, at least about 7 days/month, at least about 8 days/month, at least about 9 days/month, at least about 10 days/month, at least about 11 days/month, at least about 12 days/month, at least about 13 days/month, at least about 14 days/month, at least about 15 days/month, at least about 16 days/month, at least about 17 days/month, at least about 18 days/month, at least about 19 days/month, at least about 20 days/month, at least about 21 days/month, at least about 22 days/month, at least about 23 days/month, at least about 24 days/month, at least about 25 days/month, at least about 26 days/month, at least about 27 days/month, at least about 28 days/month, at least about 29 days/month, at least about 30 days/month, and at least about 31 days/month.

An aerosolized composition, e.g., a composition comprising (i) a first moiety comprising a ligand that interacts with a cell that participates in infection healing to concentrate the first moiety on or in the cell, linked to (ii) a second moiety comprising an antimicrobial agent, can be administered with a frequency of about 1, 2, 3, 4, or more times daily, 1, 2, 3, 4, 5, 6, 7 or more times weekly, 1, 2, 3, 4, 5, 6, 7, 8, 9, 10 or more times monthly. In certain embodiments, the compositions are administered twice daily.

In certain embodiments, the aerosol composition, such as a composition comprising (i) a first moiety comprising a ligand that interacts with a cell that participates in infection healing to concentrate the first moiety on or in the cell, linked to (ii) a second moiety comprising an antimicrobial agent, can be administered once daily, twice daily, three times daily, or four times daily. In certain embodiments, the aerosol composition, such as a composition comprising (i) a first moiety comprising a ligand that interacts with a cell that participates in infection healing to concentrate the first moiety on or in the cell, linked to (ii) a second moiety comprising an antimicrobial agent, is administered once daily. In certain embodiments, the aerosol compostion is administered twice daily. In certain embodiments, the aerosolized composition is delivered more than twice daily. In certain embodiments, the aerosol composition can be administered for a period of at least 1, 2, 3, 4, 5, 6, 8, 10, 12, 14, 16, 18, 20, 22, 24, 26, 28, or 30 days. In certain embodiments, the aerosol composition can be administered for about 14 days. In particular embodiments the aerosol composition is administered daily for 14 days. In certain embodiments, composition treatment is cycled, for example a composition is delivered in a time period as above, then treatment is stopped for a suitable amount of time, e.g., at least 1, 2, 3, 4, 5, 6, 8, 10, 12, 14, 16, 18, 20, 22, 24, 26, 28, or 30 days, then treatment is resumed, e.g., for a period as described herein. In certain embodiments, e.g., treatment of CF, a composition is delivered in a 28 day on/28 day off cycle.

The daily dosage of the composition can depend on the subject and disease state being treated, the severity of the affliction, the manner and schedule of administration, and the judgment of the prescribing physician; for example, a likely dose range for aerosol administration of a composition would be about 20 to 800 mg per day, where the dosage is calculated based on the antimicrobial, e.g., antibiotic. A daily aerosol dose of a composition can be from about 0.1 to 10 mg/kg of body weight, for example about 0.20 to 8.0 mg/kg of body weight, such as 0.4 to 6.0 mg/kg of body weight. Thus, for administration to a 70 kg person, the dosage range would be 7.0 to 840.0 mg per day, such as 14.0 to 470.0 mg per day, such as 28.0 to 350 mg per day.

The dosage of a composition per administration can be any suitable dosage.

The amount of antimicrobial, e.g., antibiotic, that can be administered (as a respirable dose, nebulizer loaded dose, and/or deposited dose) can include at least about 5 mg, 10 mg, 20 mg, about 30 mg, about 40 mg, about 50 mg, about 60 mg, about 70 mg, about 80 mg, about 90 mg, about 100 mg, about 110 mg, about 120 mg, about 125 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or 800 mg and/or not more than about 10 mg, 20 mg, 30 mg, about 40 mg, about 50 mg, about 60 mg, about 70 mg, about 80 mg, about 90 mg, about 100 mg, about 110 mg, about 120 mg, about 125 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, or about 900 mg.

For an adult, the following dosages per administration may be used

In certain embodiments, the dosage of a composition per administration of the aerosolized composition, in terms of antimicrobial, e.g., antibiotic, delivered, is 1-100, 1-80, 1-70, 2-60, 5-50, 10-30, or 20 mg.

In certain embodiments, the dosage of a composition per administration of the aerosolized composition, in terms of antimicrobial, e.g., antibiotic, delivered, is 1-100, 1-90, 1-80, 2-70, 5-60, 20-40, or 30 mg.

In certain embodiments, the dosage of a composition per administration of the aerosolized composition, in terms of antimicrobial, e.g., antibiotic, delivered, is 1-110, 1-100, 1-90, 2-80, 5-70, 40-60, or 50 mg.

In certain embodiments, the dosage of a composition per administration of the aerosolized composition, in terms of antimicrobial, e.g., antibiotic, delivered, is 10-140, 20-130, 40-120, 50-110, 60-100, 70-90, or 80 mg.

In certain embodiments, the dosage of a composition per administration of the aerosolized composition, in terms of antimicrobial, e.g., antibiotic, delivered, is 40-160, 50-150, 60-140, 70-130, 80-120, 90-110, or 100 mg.

In certain embodiments, the dosage of a composition per administration of the aerosolized composition, in terms of antimicrobial, e.g., antibiotic, delivered, is 40-200, 60-180, 70-170, 80-160, 90-150, 100-140, 110-130, or 120 mg.

In certain embodiments, the dosage of a composition per administration of the aerosolized composition, in terms of antimicrobial, e.g., antibiotic, delivered, is 80-220, 90-210, 100-200, 110-190, 120-180, 130-170, 140-160, or 150 mg.

In certain embodiments, the dosage of a composition per administration of the aerosolized composition, in terms of antimicrobial, e.g., antibiotic, delivered, is 90-210, 100-220, 110-210, 120-200, 130-190, 140-180, 150-170, or 160 mg.

In certain embodiments, the dosage of a composition per administration of the aerosolized composition, in terms of antimicrobial, e.g., antibiotic, delivered, is 100-140, 110-130, 120-220, 130-210, 140-200, 150-190, 160-180, or 170 mg.

In certain embodiments, the dosage of a composition per administration of the aerosolized composition, in terms of antimicrobial, e.g., antibiotic, delivered, is 110-250, 120-240, 130-230, 140-220, 150-210, 160-200, 170-190, or 180 mg.

In certain embodiments, the dosage of a composition per administration of the aerosolized composition, in terms of antimicrobial, e.g., antibiotic, delivered, is 120-260, 130-250, 140-240, 150-230, 160-220, 170-210, 180-200, or 190 mg.

In certain embodiments, the dosage of a composition per administration of the aerosolized composition, in terms of antimicrobial, e.g., antibiotic, delivered, is 50-400, 100-300, 120-270, 150-250, 160-240, 170-230, 180-220, 190-210, or 200 mg.

In certain embodiments, the dosage of a composition per administration of the aerosolized composition, in terms of antimicrobial, e.g., antibiotic, delivered, is 40-390, 100-320, 120-300, 150-270, 170-250, 180-240, 190-230, 200-220, or 210 mg.

In certain embodiments, the dosage of a composition per administration of the aerosolized composition, in terms of antimicrobial, e.g., antibiotic, delivered, is 50-390, 100-340, 130-310, 160-280, 180-260, 190-250, 200-240, 210-230, 220 mg.

In certain embodiments, the dosage of a composition per administration of the aerosolized composition, in terms of antimicrobial, e.g., antibiotic, delivered, is 50-400, 100-350, 140-320, 170-290, 190-270, 200-260, 210-250, 220-240, or 230 mg.

In certain embodiments, the dosage of a composition per administration of the aerosolized composition, in terms of antimicrobial, e.g., antibiotic, delivered, is 50-430, 100-380, 130-350, 160-320, 180-300, 200-280, 210-270, 220-260, 230-250, or 240 mg.

In certain embodiments, the dosage of a composition per administration of the aerosolized composition, in terms of antimicrobial, e.g., antibiotic, delivered, is 100-400, 130-370, 160-340, 180-320, 200-300, 210-290, 220-280, 230-270, 240-260, or 250 mg.

In certain embodiments, the dosage of a composition per administration of the aerosolized composition, in terms of antimicrobial, e.g., antibiotic, delivered, per administration of the aerosolized composition, is 100-420, 120-400, 150-370, 170-350, 200-320, 220-300, 230-290, 240-280, 250-270, or 260 mg.

In certain embodiments, the dosage of a composition per administration of the aerosolized composition, in terms of antimicrobial, e.g., antibiotic, delivered, is 100-440, 150-390, 180-360, 210-330, 230-310, 240-300, 250-290, 260-280, or 270 mg.

In certain embodiments, the dosage of a composition per administration of the aerosolized composition, in terms of antimicrobial, e.g., antibiotic, delivered, is 110-450, 140-420, 170-390, 200-360, 220-340, 240-320, 250-310, 260-300, 270-290, or 280 mg.

In certain embodiments, the dosage of a composition per administration of the aerosolized composition, in terms of antimicrobial, e.g., antibiotic, delivered, is 120-460, 150-430, 180-400, 210-370, 230-350, 250-330, 260-320, 270-310, 280-300, or 290 mg.

In certain embodiments, the dosage of a composition per administration of the aerosolized composition, in terms of antimicrobial, e.g., antibiotic, delivered, is 70-430, 100-400, 120-380, 140-360, 160-340, 170-330, 180-320, 190-310, 190-310, 195-305, or 300 mg.

In certain embodiments, the dosage of a composition per administration of the aerosolized composition, in terms of antimicrobial, e.g., antibiotic, delivered, is 100-520, 170-450, 200-420, 230-390, 250-370, 270-350, 280-340, 290-330, 300-320, 310 mg.

In certain embodiments, the dosage of a composition per administration of the aerosolized composition, in terms of antimicrobial, e.g., antibiotic, delivered, is 190-450, 220-420, 250-390, 270-370, 290-350, 300-340, 310-330, or 320 mg.

In certain embodiments, the dosage of a composition per administration of the aerosolized composition, in terms of antimicrobial, e.g., antibiotic, delivered, is 200-460, 240-420, 270-390, 290-370, 300-360, 310-350, 320-340, or 330 mg.

In certain embodiments, the dosage of a composition per administration of the aerosolized composition, in terms of antimicrobial, e.g., antibiotic, delivered, is 200-490, 240-450, 270-410, 290-390, 310-370, 320-360, 330-350, or 340 mg.

In certain embodiments, the dosage a composition per administration of the aerosolized composition, in terms of antimicrobial, e.g., antibiotic, delivered, is 200-500, 240-460, 270-430, 300-400, 320-380, 330-370, 340-360, or 350 mg.

In certain embodiments, the dosage of a composition per administration of the aerosolized composition, in terms of antimicrobial, e.g., antibiotic, delivered, is 200-520, 230-490, 260-460, 290-430, 310-410, 330-390, 340-380, 350-370, or 360 mg.

In certain embodiments, the dosage a composition per administration of the aerosolized composition, in terms of antimicrobial, e.g., antibiotic, delivered, is 250-490, 290-450, 310-430, 330-410, 340-400, 350-390, 360-380, or 370 mg.

In certain embodiments, the dosage of a composition per administration of the aerosolized composition, in terms of antimicrobial, e.g., antibiotic, delivered, is 230-530, 270-490, 300-460, 320-440, 340-420, 350-410, 360-400, 370-390, or 380 mg.

In certain embodiments, the dosage of a composition per administration of the aerosolized composition, in terms of antimicrobial, e.g., antibiotic, delivered, is 250-530, 300-480, 330-450, 350-430, 360-420, 370-410, 380-400, or 390 mg.

In certain embodiments, the dosage of a composition per administration of the aerosolized composition, in terms of antimicrobial, e.g., antibiotic, delivered, is 260-540, 300-500, 330-470, 350-450, 370-430, 380-420, 390-410, or 400 mg.

In certain embodiments, the subject is an adult and the dosage per administration is 50-500, 100-450, 200-400, 250-350, 280-320, 290-310, or 300 mg; or 100-600, 200-500, 300-400, 320-380, 340-360, or 350 mg; or 100-700, 200-600, 300-500, 350-450, 380-420, 390-410, or 400 mg; or 50-600, 100-500, 200-400, 230-270, 240-260, or 250 mg; or 50-400, 100-300, 150-250, 180-220, 190-210, or 200 mg.

In certain embodiments, the subject is a pediatric patient and, as appropriate, the dosage may be reduced, e.g., to less than 90, 80, 70, 60, 50, 40, 30, or 20% of the adult dose.

In certain embodiments, a respirable drug dose (RDD) of at least 5, 10, 20, 100, 125, or 150 mg of antimicrobial, e.g., antibiotic, is administered to the lung. In certain embodiments, a loaded dose of at least 20, 40, 60, 80, 100, 200, 250, 300, 350, or 400 mg of antimicrobial, e.g., antibiotic, is aerosolized.

The aerosol can be administered to the lungs in less than 10 minutes, 5 minutes, 4 minutes, 3 minutes, 2 minutes, or 1 minute.

In certain embodiments, administration of the aerosolized composition achieves a maximum lung sputum concentration of the antimicrobial, e.g., antibiotic, (Cₘₐₓ) of at least 1200, 1700, 2000, 3000, or 4000 mg/L, for example at least 1200 mg/L and a lung sputum area under the curve (AUC) of at least 1500, 1700, 2000, 3000, or 4000 h·mg/L, for example at least 1500 h·mg/L. In certain of these embodiments, the composition comprises a divalent or trivalent cation, or a combination thereof, e.g., magnesium, calcium, zinc, copper, aluminum, or iron, or a combination thereof, such as magnesium, in some cases in the form of magnesium chloride, e.g., at a concentration of 50-400 mM. The antimicrobial, e.g., antibiotic, concentration in the composition can be 10-100, or 10-200, or 20-100, or 20-80, or 50-200 mg/mL. In certain embodiments, the composition comprises no lactose. In certain embodiments, the composition comprises a divalent or trivalent cation, or combination thereof, such as a divalent cation, e.g., magnesium, at a concentration of 50-400, 100-300, or 150-250 mM. In certain embodiments, the composition comprises an antimicrobial, e.g., an antibiotic, at a concentration of 10-100, 10-200, 20-100, or 20-80, 50-200, 75-150, or 90-125 mg/mL. In certain embodiments, the osmolarity of the composition is 200-800, 300-600, or 350-425 mOsmol/kg. In certain embodiments, the pH of the composition is 5-8, 5-7, 5-6.5, or 5.5-6.5. In certain embodiments the composition comprises a antimicrobial, e.g., antibiotic, concentration of 20-80 mg/ml, or 20-40 mg/ml, or 90-110 mg/ml, a magnesium chloride concentration of 175-225 mM, a pH of 5-7; an osmolarity of 300-500 mOsmol/kg, and, optionally, lacks lactose

In certain embodiments the method comprises administering a composition comprising (i) a first moiety comprising a ligand that interacts with a cell that participates in infection healing to concentrate the first moiety on or in the cell, linked to (ii) a second moiety comprising an antimicrobial agent, such as an antibiotic, to the subject, e.g., human, to achieve a concentration in a lung of the subject of at least 5, 10, 20, 25, 27, 32, 35, 40, 45, 50, 70, 100, 200, 500, 800, 1000, 1200, or 1500 μg/ml of the antimicrobial, e.g., antibiotic, wherein the composition is administered as a aerosol. In certain embodiments the aerosol comprises a divalent or trivalent cation or combination thereof. In certain embodiments, the aerosol comprises greater than 50 mg/ml of the antimicrobial, e.g., antibiotic, and, in certain embodiments, a divalent or trivalent cation, or combination thereof, e.g., magnesium, such as magnesium supplied by magnesium chloride, has a pH of 5-8, 5-7.5, 5-7, 5.5-8, 5.5-7.5, 5.5-7, or 5.5-6.5, and an osmolality of 100-1200, 200-1000, 300-900, or 350-750 mOsmol/kg

In certain embodiments the method comprises administering a composition comprising (i) a first moiety comprising a ligand that interacts with a cell that participates in infection healing to concentrate the first moiety on or in the cell, linked to (ii) a second moiety comprising an antimicrobial agent, such as an antibiotic, to a subject, e.g., human suffering from a bacterial infection caused by at least one type of bacteria, wherein the bacteria is exposed to at least 0.01, 0.05, 0.07, 0.1, 0.15, 0.2, 0.3, 0.4, 0.5, 0.6, 0.65, 0.7, 0.75, 0.8, 0.85, 0.9, 1, 1.2, 1.5, 1.7, 2, 2.5, 3, 4, 5, 7, or 10 mg/L of the antimicrobial, e.g., antibiotic, wherein the composition is administered as an aerosol. In certain embodiments the aerosol comprises a divalent or trivalent cation or combination thereof. In certain embodiments, the aerosol comprises greater than 50 mg/ml of the antimicrobial, e.g., antibiotic, and, in certain embodiments, a divalent or trivalent cation, or combination thereof, e.g., magnesium, such as magnesium supplied by magnesium chloride, has a pH of 5-8, 5-7.5, 5-7, 5.5-8, 5.5-7.5, 5.5-7, or 5.5-6.5, and an osmolality of 100-1200, 200-1000, 300-900, or 350-750 mOsmol/kg. In certain embodiments, no other antibiotics are administered by inhalation; in certain embodiments, no other antibiotics are administered. In certain embodiments, at least 5, 10, 20, 50, 70, 100, 120, 150, 170, 200, 220, 250, 270, or 300 mg of the antimicrobial, e.g., antibiotic, is administered.

In certain embodiments aerosolized composition is repeatedly administered to a subject, e.g., human, where repeated administration does not result in an incidence of arthralgia. In certain embodiments, administering is repeated at least once daily for 14 days, at least once daily for 28 days, and at least once daily for 35 days. In certain embodiments, administering is repeated at least twice daily for at least 14 days, at least twice daily for at least 28 days, and at least twice daily for at least 35 days.

In certain embodiments, the composition is in a unit dosage form, such as vial containing a liquid, solid to be suspended, dry powder, lyophilizate, or other composition. In these embodiments, the composition may contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like.

In certain embodiments, the particles of the aerosol containing a composition comprising (i) a first moiety comprising a ligand that interacts with a cell that participates in infection healing to concentrate the first moiety on or in the cell, linked to (ii) a second moiety comprising an antimicrobial agent, such as an antibiotic, have a mass median aerodynamic diameter of 2-5 with a geometric standard deviation less than or equal to about 2.5 microns.

In certain embodiments, the particles of the aerosol containing a composition comprising (i) a first moiety comprising a ligand that interacts with a cell that participates in infection healing to concentrate the first moiety on or in the cell, linked to (ii) a second moiety comprising an antimicrobial agent, such as an antibiotic, have a mass median aerodynamic diameter of 2.5-4.5 microns with a geometric standard deviation less than or equal to 1.8 microns.

In certain embodiments, the particles of the aerosol containing a composition comprising (i) a first moiety comprising a ligand that interacts with a cell that participates in infection healing to concentrate the first moiety on or in the cell, linked to (ii) a second moiety comprising an antimicrobial agent, such as an antibiotic, have a mass median aerodynamic diameter of 2.8-4.3 microns with a geometric standard deviation less than or equal to about 2 microns.

In certain embodiments, the method also includes producing the aerosol with a vibrating mesh nebulizer. In some such embodiments, the vibrating mesh nebulizer is a PARI E-FLOW™ nebulizer.

In certain embodiments, the amount of a composition comprising (i) a first moiety comprising a ligand that interacts with a cell that participates in infection healing to concentrate the first moiety on or in the cell, linked to (ii) a second moiety comprising an antimicrobial agent, such as an antibiotic, administered to the lung, in terms of antimicrobial, e.g., antibiotic, is at least about 5 mg, at least about 10 mg, at least about 15 mg, at least about 20 mg, at least about 100 mg, at least about 125 mg, and at least about 150 mg.

In certain embodiments, at least about 20, 40, 60, 80, or 100 mg of the antimicrobial, e.g., antibiotic, in the aerosol is administered to the lung in less than about 10 minutes, less than about 5 minutes, less than about 3 minutes, less than about 2 minutes.

In certain embodiments the treatment includes administering an additional active agent, for example one or more antibiotics, bronchodilators, anticholinergics, glucocorticoids, eicosanoid inhibitors, CFTR modulators, agents to restore airway surface liquid, anti-inflammatory agents, or combinations thereof. The coadministration can comprise inhalation of the agent. The agent may be administered as part of the aerosolized composition, separately, or a combination thereof. In certain embodiments, the antibiotic can include tobramycin, aztreonam, ciprofloxacin, azithromycin, tetracycline, quinupristin, linezolid, vancomycin, and chloramphenicol, colisitin or combinations thereof. In some embodiments, the bronchodilator can include salbutamol, levosalbuterol, terbutaline, fenoterol, terbutlaine, pirbuterol, procaterol, bitolterol, rimiterol, carbuterol, tulobuterol, reproterol, salmeterol, formoterol, arformoterol, bambuterol, clenbuterol, indacterol, theophylline, roflumilast, cilomilast, or combinations thereof. In certain embodiments, the anticholinergic can be ipratropium, tiotropium, and combinations thereof. In certain embodiments, the glucocorticoid can include prednisone, fluticasone, budesonide, mometasone, ciclesonide, beclomethasone, or combinations thereof. In certain embodiments, the eicosanoid inhibitor can include montelukast, pranlukast, zafirlukast, zileuton, ramatroban, seratrodast, or combinations thereof. In certain embodiments the CFTR modulator includes VX-770, atluren, VX-809, or combinations thereof. In certain embodiments, the agent to restore airway surface liquid includes denufosol, mannitol, GS-9411, SPI-8811, or combinations thereof. In certain embodiments, the anti-inflammatory agent includes ibuprofen, sildenafil, simavastatin, or combinations thereof. In certain embodiments, co-administering comprises inhaling the additional active agent. In certain embodiments, e.g., the treatment of CF, the additional active ingredient comprises mannitol.

In certain embodiments, the aerosol therapy with a composition comprising (i) a first moiety comprising a ligand that interacts with a cell that participates in infection healing to concentrate the first moiety on or in the cell, linked to (ii) a second moiety comprising an antimicrobial agent, such as an antibiotic may be administered as a treatment or prophylaxis in combination or alternating therapeutic sequence with other aerosol, oral or parenteral antibiotics. Any suitable antibiotic may be used, e.g., tobramycin and/or other aminoglycoside, aztreonam, carumonam and/or tigemonam and/or other beta or mono-bactam, ciprofloxacin and/or other fluoroquinolones, azithromycin and/or other macrolides or ketolides, tetracycline and/or other tetracyclines, quinupristin and/or other streptogramins, linezolid and/or other oxazolidinones, vancomycin and/or other glycopeptides, and/or chloramphenicol and/or other phenicols, and/or colisitin and/or other polymyxins. In certain embodiments, the antibiotic can include quinolones, tetracyclines, glycopeptides, aminoglycosides, beta-lactams, rifamycins, macrolides/ketolides, oxazolidinones, coumermycins, chloramphenicol, streptogramins, trimethoprim, sulfamethoxazole, or polymyxins. In some embodiments, any of the foregoing antibiotics can be administered by any acceptable method or route, for example, by aerosol, orally or parenterally.

### Beta-Lactam Antibiotics

Beta-lactam antibiotics suitable for administration by inhalation in a composition comprising (i) a first moiety comprising a ligand that interacts with a cell that participates in infection healing to concentrate the first moiety on or in the cell, linked to (ii) a second moiety comprising an antimicrobial agent, such as an antibiotic, include, but are not limited to, imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephaacetrile, cephalexin, cephaloglycin, cephaloridine, cephalothin, cephapirin, cephradine, cefmetazole, cefoxitin, cefotetan, azthreonam, carumonam, flomoxef, moxalactam, amidinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, and LY206763.

### Macrolides

Macrolides suitable for administration by inhalation in a composition comprising (i) a first moiety comprising a ligand that interacts with a cell that participates in infection healing to concentrate the first moiety on or in the cell, linked to (ii) a second moiety comprising an antimicrobial agent, such as an antibiotic, include, but are not limited to, azithromycin, clarithromycin, erythromycin, oleandomycin, rokitamycin, rosaramicin, roxithromycin, and troleandomycin.

### Ketolides

Ketolides suitable for administration by inhalation in a composition comprising (i) a first moiety comprising a ligand that interacts with a cell that participates in infection healing to concentrate the first moiety on or in the cell, linked to (ii) a second moiety comprising an antimicrobial agent, such as an antibiotic, include, but are not limited to, telithromycin and cethromycin.

### Quinolones

Quinolones suitable for administration by inhalation in a composition comprising (i) a first moiety comprising a ligand that interacts with a cell that participates in infection healing to concentrate the first moiety on or in the cell, linked to (ii) a second moiety comprising an antimicrobial agent, such as an antibiotic, include, but are not limited to, amifloxacin, cinoxacin, ciprofloxacin, enoxacin, fleroxacin, flumequine, lomefloxacin, nalidixic acid, norfloxacin, ofloxacin, levofloxacin, oxolinic acid, pefloxacin, rosoxacin, temafloxacin, tosufloxacin, sparfloxacin, clinafloxacin, moxifloxacin; gemifloxacin; garenofloxacin; PD131628, PD138312, PD140248, Q-35, AM-1155, NM394, T-3761, rufloxacin, OPC-17116, DU-6859a (see, e.g., Sato, K. et al., 1992, Antimicrob Agents Chemother. 37:1491-98), and DV-7751a (see, e.g., Tanaka, M. et al., 1992, Antimicrob. Agents Chemother. 37:2212-18).

### Tetracyclines, Glycylcyclines and Oxazolidinones

Tetracyclines, glycylcyclines, and oxazolidinones suitable for administration by inhalation in a composition comprising (i) a first moiety comprising a ligand that interacts with a cell that participates in infection healing to concentrate the first moiety on or in the cell, linked to (ii) a second moiety comprising an antimicrobial agent, such as an antibiotic, include, but are not limited to, chlortetracycline, demeclocycline, doxycycline, lymecycline, methacycline, minocycline, oxytetracycline, tetracycline, tigecycline, linezolide, and eperozolid.

### Aminoglycosides

Aminoglycosides suitable for administration by inhalation in a composition comprising (i) a first moiety comprising a ligand that interacts with a cell that participates in infection healing to concentrate the first moiety on or in the cell, linked to (ii) a second moiety comprising an antimicrobial agent, such as an antibiotic, include, but are not limited to amikacin, arbekacin, butirosin, dibekacin, fortimicins, gentamicin, kanamycin, meomycin, netilmicin, ribostamycin, sisomicin, spectinomycin, streptomycin, and tobramycin.

### Lincosamides

Lincosamides suitable for administration by inhalation in a composition comprising (i) a first moiety comprising a ligand that interacts with a cell that participates in infection healing to concentrate the first moiety on or in the cell, linked to (ii) a second moiety comprising an antimicrobial agent, such as an antibiotic, include, but are not limited to, clindamycin and lincomycin.

### Streptogramins

Streptogramins suitable for administration by inhalation in a composition comprising (i) a first moiety comprising a ligand that interacts with a cell that participates in infection healing to concentrate the first moiety on or in the cell, linked to (ii) a second moiety comprising an antimicrobial agent, such as an antibiotic, include, but are not limited to quinupristin.

### Glycopeptides

Glycopeptides suitable for administration by inhalation in a composition comprising (i) a first moiety comprising a ligand that interacts with a cell that participates in infection healing to concentrate the first moiety on or in the cell, linked to (ii) a second moiety comprising an antimicrobial agent, such as an antibiotic, include, but are not limited to vancomycin.

### Polymyxins

Polymyxins suitable for administration by inhalation in a composition comprising (i) a first moiety comprising a ligand that interacts with a cell that participates in infection healing to concentrate the first moiety on or in the cell, linked to (ii) a second moiety comprising an antimicrobial agent, such as an antibiotic, include but are not limited to colisitin.

Additional antibiotics suitable for administration by inhalation in a composition comprising (i) a first moiety comprising a ligand that interacts with a cell that participates in infection healing to concentrate the first moiety on or in the cell, linked to (ii) a second moiety comprising an antimicrobial agent, such as an antibiotic, include fosfomycin, penicillins, cephalosporins, carbapenems, penems, and carbacephems.

In certain embodiments, treating a subject, e.g., human, suffering from a pulmonary bacterial infection with aerosolized composition comprising (i) a first moiety comprising a ligand that interacts with a cell that participates in infection healing to concentrate the first moiety on or in the cell, linked to (ii) a second moiety comprising an antimicrobial agent, such as an antibiotic, can result in a clinically measurable response, such as a reduction in pulmonary infection, an improvement in a pulmonary function characteristic, such as an improvement in forced expiratory volume (FEV), FEV₁ (forced expiratory volume in 1 second), and FEF 25-75 (forced expiratory flow 25-75%), reducing the need for other inhaled or systemic antibiotics, decreasing frequency, severity, duration, and/or likelihood of exacerbations.

A reduction in a pulmonary infection can be measured using any suitable method. For example, in a pulmonary infection comprising one or more organisms, a reduction in the density of the organism can be measured. In some embodiments, treatment can achieve a reduction in the density of an organism by at least about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%. In some embodiments, treatment can achieve a reduction in the density of an organism by at least about 85%, about 86%, about 87%, about 88%, about 89%, about 90%, about 91%, about 92%, about 93%, about 94%, about 95%, about 96%, about 97%, about 98%, about 99%, and about 100%.

The density of an organism can be measured in a sample taken from a subject, for example, bronchial alveolar lavage, sputum, or serum. In certain embodiments the density of an organism can be reduced by at least about 0.1, about 0.2, about 0.3, about 0.4, about 0.5, about 0.6, about 0.7, about 0.8, about 0.8, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5 log₁₀ CFU/g sputum, or more.

Certain embodiments of the methods and compositions described herein can include achieving an improvement in a pulmonary function parameter. Examples of such parameters can include FEV (forced expiratory volume), FEV₁ (forced expiratory volume in 1 second), and/or FEF 25-75 (forced expiratory flow 25-75%). In certain embodiments, the FEV₁ of a subject can be increased using the methods and compositions described herein, by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, and more. In certain embodiments, the FEV₁ of a subject can be increased using the methods and compositions described herein, by at least about 0.01 L, 0.02 L, 0.03 L, 0.04 L, and 0.05 L, and by at least about 0.1 L, 0.2 L, 0.3 L, 0.4 L, 0.5 L, 0.6 L, 0.7 L, 0.8 L, 0.9 L, 1.0 L, and more.

In certain embodiments, the FEF 25-75 of a subject can be increased using the methods and compositions described herein, by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, and 25%. In certain embodiments, the FEF 25-75 of a subject can be increased using the methods and compositions described herein, by at least about 0.01 L, 0.02 L, 0.03 L, 0.04 L, and 0.05 L, and by at least about 0.1 L, 0.2 L, 0.3 L, 0.4 L, 0.5 L, 0.6 L, 0.7 L, 0.8 L, 0.9 L, 1.0 L, or more.

Certain embodiments of the methods and compositions described herein can include reducing the need for a subject to need other inhaled or systemic antibiotics, such as anti-pseudomonal antimicrobials. Such a reduction can be measured by any suitable method, for example, by the increase in time to need other inhaled or systemic antibiotics. A reduction in such a need can be measured by a variety of statistical means. For example, hazard ratios may be used in a survival analysis. In some embodiments, the hazard ratio is less than about 1.0, 0.9, 0.8, 0.7, 0.6, 0.5, 0.4, 0.3, 0.2, 0.1, and less.

Some embodiments of the methods and compositions described herein can include decreasing the frequency of exacerbations, the severity of exacerbations, the duration of exacerbations, and/or the likelihood that an exacerbation will occur. An exacerbation can be defined by any of several methods and criteria provided by such methods. In certain embodiments, a patient can concurrently meet at least 4 symptoms/signs of the Fuchs definition of an exacerbation (Fuchs H J, et al. Effect of aerosolized recombinant human DNase on exacerbations of respiratory symptoms and on pulmonary function in patients with cystic fibrosis. N Engl J Med 1994; 331:637-642). The symptoms/signs defined by the Fuchs criteria include: change in sputum; new or increased hemoptysis; increased cough; increased dyspnea; malaise, fatigue or lethargy; temperature above 38° C.; anorexia or weight loss; sinus pain or tenderness; change in sinus discharge; change in physical examination of the chest; decrease in pulmonary function by 10% or more from a previously recorded value; and radiographic changes indicative of pulmonary infection.

In certain embodiments, a patient with an improved exacerbation profile can have at least 1, at least 2, at least 3, and at least 4 of the following signs/symptoms, where changes can be relative to a patient's typical experience, for example daily experience, and weekly experience. (1) Change in sputum, e.g., for sputum production: patients have no change, a little less or much less amounts of sputum when coughing up, or for change in sputum appearance: for sputum thickness, patients have a little thinner or much thinner sputum; for sputum color, patients have a better color of sputum (better increases from brown→green→yellow→clear). (2) Hemoptysis, e.g., patients have a little decrease or a large decrease in the amount of blood coughed up. (3) Cough, e.g., for intensity of cough, patients have a little lighter, or much lighter coughs; for frequency of cough, patients cough a little less often or much less often. (4) Dyspnea, e.g., for dyspnea with exertion, patients breathe a little easier or much easier when performing daily activities. (5) Malaise, fatigue or lethargy, e.g., patients have a little more energy or much more energy, and/or patients perform daily activities, e.g., climbing stairs, a little easier, or much easier. (6) Temperature, e.g., patients have normal healthy temperature e.g., about 37° C., or patients have no recent history of fever. (7) Anorexia or weight loss, e.g., patients have no change in weight, or a little weight gain, and/or patients have a little increase in appetite (8) Sinus pain or tenderness, e.g., patient has no sinus pain or tenderness, or less sinus pain or tenderness. (9) Change in sinus discharge, e.g., patients have better sinus discharge (a decrease in thickness and/or better color). (10) Change in physical examination of the chest, e.g., patients have improved signs on examination of the chest and may report for example, a little decrease chest congestion, or a large decrease in chest congestion. (11) Pulmonary function by 10% or more from a previously recorded value, e.g., patients have improved pulmonary function in pulmonary function tests. (12) Radiographic changes indicative of pulmonary infection, e.g. patients show improved radiographic changes indicating reduced pulmonary infection.

In certain embodiments, exercise tolerance and/or absenteeism from scheduled events, e.g., school or work can be measured as signs/symptoms of exacerbations.

Summaries of such characteristics are known in the art; see, e.g., Table 1 of U.S. Pat. No. 10,792,289.

In certain embodiments, the treatment results in one, two, three, four, five, or six of an increase in a CFQ-R respiratory domain greater than 1; a reduction in the density of bacteria by at least 40%; an increase in FEV₁ of at least 2%; an increase in FEF 25-75 of at least 5%; a hazard ratio less than 1.0; a dose-normalized serum Cₘₐₓ of antimicrobial, e.g., antibiotic greater than 2 μg/L/mg; and/or a dose-normalized serum AUC of antimicrobial, e.g., antibiotic, of at least 20 (ng·h/L) mg.

Some embodiments of any of the above methods include administering a composition comprising (i) a first moiety comprising a ligand that interacts with a cell that participates in infection healing to concentrate the first moiety on or in the cell, linked to (ii) a second moiety comprising an antimicrobial agent, such as an antibiotic, in combination with a divalent or trivalent cation, or combination thereof, in a dosage amount, administration schedule, and/or method of administration sufficient to achieve the above recited outcomes.

Treatment of Pulmonary Infection in a Human with Cystic Fibrosis

In certain embodiments, provided herein is a method of treating a human subject with cystic fibrosis who is suffering from a bacterial infection by administering a therapeutically effective amount of an aerosolized composition, such as one of the compositions described herein, e.g., a liquid formulation such as an aqueous formulation, a dry powder formulation, or a liposomal formulation. In certain embodiments, the administration is prophylactic. The composition can be, e.g., a composition comprising (i) a first moiety comprising a ligand that interacts with a cell that participates in infection healing to concentrate the first moiety on or in the cell, linked to (ii) a second moiety comprising an antimicrobial agent. Bacteria to be treated, specific compositions, frequency of dosing, dosage amounts, duration of aerosol administration, concentrations or effects to be achieved, particle size of the aerosols, additional treatment modalities, indications of effective treatment, and the like, may be any suitable embodiment, as described above, “Treatment of pulmonary bacterial infections.”

In certain embodiments, the bacterial infection can be any bacterial infection susceptible to treatment with the antimicrobial agent. In certain embodiments, the bacterial infection is an antibiotic-resistant bacterial infection. In certain embodiments, the bacterial infection comprises Pseudomonas bacteria, such as is Pseudomonas aeruginosa, Pseudomonas fluorescens, Pseudomonas acidovorans, Pseudomonas alcaligenes, Pseudomonas putida, or a combination of two or more thereof. In certain embodiments, the infection is a Pseudomonas aeruginosa infection. In certain embodiments, the bacterial infection is a methicillin resistant Staphylococcus aureus (MRSA) infection. In certain embodiments, the infection is a Streptocossus pneumonia (Sp) infection. In certain embodiments, the infection comprises one or more Mycobacterium, such as one or more of Mycobacterium tuberculosis, Mycobacterium avium, Mycobacterium intracellulare, or Mycobacterium leprae, for example Mycobacterium avium or Mycobacterium intracellulare. In certain embodiments, the bacterial infection comprises Haemophilus influenzae. In certain embodiments the bacterial infection comprises Haemophilus parainfluenza. In certain embodiments the bacterial infection comprises Moraxella catarrhalis. Other bacteria that can be treated include those described in Treatment of pulmonary bacterial infections”.

In certain embodiments, the aerosol composition can be administered daily, or twice daily. In certain embodiments, the aerosol composition can be administered for a period of at least 1 day, 3 days, 5 days, 10 days, 15 days, 20 days, or 30 days. In certain embodiments, the aerosol composition can be administered for about 14 days. In particular embodiments the aerosol composition is administered daily for 14 days. In certain embodiments, the aerosol composition is delivered for a period of 28 days on, 28 days off. In certain embodiments, the subject is an adult. In certain embodiments, the subject is a pediatric patient. In certain embodiments, the subject has an age less than about 18 years, less than about 17 years, less than about 16 years, less than about 15 years, less than about 14 years, less than about 13 years, less than about 12 years, less than about 11 years, less than about 10 years, less than about 9 years, less than about 8 years, less than about 7 years, less than about 6 years, less than about 5 years, less than about 4 years, less than about 3 years, less than about 2 years, and less than about 1 year. Dosage will generally depend on the age and/or weight of the subject. In certain embodiments, the subject is an adult and the dosage per administration is 10-100, 10-200, 20-100, 20-80, 50-500, 100-450, 200-400, 250-350, 280-320, 290-310, or 300 mg; or 100-600, 200-500, 300-400, 320-380, 340-360, or 350 mg; or 100-700, 200-600, 300-500, 350-450, 380-420, 390-410, or 400 mg; or 50-600, 100-500, 200-400, 230-270, 240-260, or 250 mg; or 50-400, 100-300, 150-250, 180-220, 190-210, or 200 mg. Other suitable dosages are as described in “Treatment of pulmonary bacterial infections” In certain embodiments, the subject is a pediatric patient and the dosage is reduced appropriately, e.g., to less than 90, 80, 70, 60, 50, 40, 30, or 20% of the adult dose.

In certain embodiments, provided is a method for treating a pulmonary infection in a human having cystic fibrosis, wherein the pulmonary infection comprises one or more Mycobacterium, comprising administering via inhalation 50-1000, 75-800, 100-500, 100-400, 200-500, 200-400, 250-350, or 300 mg of composition twice daily for 28 days to the human having cystic fibrosis to treat the Mycobacterium pulmonary infection. The composition can in an aerosol of a solution comprising composition at a concentration from about 10, 20, 30, 40, 50, 60, 70, 80, or 90 mg/ml to about 20, 30, 40, 50, 60, 70, 80, 90, 100 or 110 mg/ml.

In certain embodiments, provided is a method for treating a pulmonary infection due to Pseudomonas, e.g., Pseudomonas aeruginosa in a subject, e.g., human, with cystic fibrosis in need thereof, the method comprising administering to the lungs of the subject with cystic fibrosis an aerosol of a solution comprising 10-500, 20-400, 20-100, 30-300, 30-100, 40-200, 50-200, 70-200, 50-150, 90-110, or 100 mg/ml of composition to treat the chronic pulmonary infection due to Pseudomonas, e.g., Pseudomonas aeruginosa.

### VII. EXAMPLES

### Example 1

In this Example, various prodrugs are developed and evaluated.

In Period 1, non-GLP chemical synthesis is used to prepare a small library of lead candidates at the sub-gram level that enter screening toward proof of concept. After screening a few promising candidate NCEs move forward to lab scale up. Preparation of gram quantities is needed to conduct the requisite non-clinical studies leading to proof of concept. In Period 2, this process is repeated for other classes of antibiotics. Lead candidates coming from Period 1 work are then be progressed forward into formulation development, scale up and GLP/GMP API manufacturing to support pre-clinical IND enabling work. Formulation work is initiated along with GMP manufacturing of the drug product. Stability, characterization, and specification development work continue through this work period to support an IND submission and ultimately clinical development.

The first step in the process is identifying acceptable transporters on the immune carrier cells and confirm the specificity of the target. Rigorous literature searches allow identification of transporters that are expressed in activated infection healing cells, e.g., immune cells or tissue repair cells, and that are suitable for a prodrug approach. Pilot experiments confirm transporter expression and prodrug strategies. The antibiotics payloads are selected from a class of antibiotics with broad spectrum of activity, preferably for Gram-negative bacteria.

Medicinal chemistry strategies (i.e., SAR) include the generation of expanded library of potential prodrugs of existing anti-infective drugs, that will increase cell distribution through the targeted transporter(s). In vitro assay in cell lines conditionally expressing the transporter of interest is used to screen the prodrugs to determine their transporter uptake. In addition, in situ experiments with freshly isolated cell carrier are used to confirm the target specificity in situ. The chemistry is optimized based on the structure activity relationship observed in the in vitro and in situ studies and the most promising lead compounds are selected for in vivo testing.

The lead compound(s) are tested in vitro using readily available screening batteries to determine ADME characteristics. This is followed by in vivo studies in animals for safety and pharmacokinetic assessment. The most promising compound(s) are tested in a battery of animal models of infectious disease to confirm the in vivo proof of concept (POC 1).

This process is applied to additional classes of transporters and antibiotics to investigate in vivo (POC 2). The most promising lead candidate is selected for further development and carried forward, e.g., to an IND.

In this Example, the proposed product is intended to improve the efficacy of currently marketed antibiotics against resistant microorganisms through a unique targeted delivery of the antibiotics into the site of infections. The development timeline is shorter as these are prodrugs of approved antibiotics. Patients with hard to treat infections such as lung infection (especially in cystic fibrosis), pneumonia and gonorrhea are able to recover faster therefore reducing mortality and reducing health care cost.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising
(i) a first moiety comprising a ligand that interacts with a cell that participates in infection healing to concentrate the first moiety on or in the cell, linked to
(ii) a second moiety comprising an antimicrobial agent.

2. The composition of claim 1 wherein the ligand comprises a structure that is concentrated in the cell by passive diffusion.

3. The composition of claim 1 wherein the ligand comprises a ligand that interacts with a target structure of the cell.

4. The composition of claim 1 wherein the cell that participates in infection healing comprises an immune cell.

5. The composition of claim 4 wherein the immune cell comprises a lymphocyte, neutrophil, or monocyte/macrophage.

6. The composition of claim 5 wherein the immune cell comprises a lymphocyte comprising a T cell, a B cell, or a natural killer (NK) cell.

7. The composition of claim 5 wherein the immune cell comprises a neutrophil or a monocyte/macrophage.

8. The composition of claim 5 wherein the immune cell comprises a neutrophil.

9. The composition of claim 1 wherein the cell that participates in infection healing comprises a tissue repair cell.

10. The composition of claim 1 wherein the tissue repair cell comprises a fibroblast.

11. The composition of claim 3 wherein the target structure is a structure on the extracellular surface of a plasma membrane of the cell.

12. The composition of claim 2 wherein the target structure is a transmembrane moiety.

13. The composition of claim 11 wherein the transmembrane moiety is a transporter.

14. The composition of claim 13 wherein the transporter is a nutrient transporter.

15. The composition of claim 13 wherein the transporter comprises an amino acid transporter, a nucleic acid transporter, a carbohydrate transporter, an organic cation transporter, a fatty acid transporter, an antioxidant transporter, or a vitamin transporter.

16. The composition of claim 15 wherein the transporter is a carbohydrate transporter comprising a glucose transporter.

17. The composition of claim 16 wherein the glucose transporter comprises a GLUT1 (SLC2A1) or a GLUT3 (SLC2A3) transporter.

18. The composition of claim 15 wherein the transporter is an amino acid transporter.

19. The composition of claim 18 wherein the amino acid transporter comprises ATB^{0,+} (SLC6A14), b^{0,+}AT (SLC7A9), or xCT (SLC7A11).

20. The composition of claim 15 wherein the transporter is an organic cation transporter.

21. The composition of claim 20 wherein the organic cation transporter is OCNT1 (SLC22A4) or OCTN2 (SLC22A5).

22. The composition of claim 15 wherein the transporter is an antioxidant transporter or a vitamin transporter.

23. The composition of claim 22 wherein the transporter is an ascorbic acid transporter.

24. The composition of claim 23 wherein the ascorbic acid transporter comprises SVCT1, SVCT2 (SLC23A2), GLUT1 or GLUT3.

25. The composition of claim 23 wherein the ligand that interacts with the target moiety comprises ascorbic acid or an ascorbic acid derivative.

26. The composition of claim 3 wherein the target structure is increased in expression in response to infection.

27. The composition of claim 1 wherein the antimicrobial agent comprises an antibacterial agent, an antiviral agent, an antifungal agent, or an antiparasitic agent.

28. The composition of claim 1 wherein the antimicrobial agent has received regulatory approval.

29. The composition of claim 27 wherein the antimicrobial agent comprises an antibacterial agent.

30. The composition of claim 29 wherein the antibacterial agent comprises a fluoroquinolone, or a beta-lactam.

31. The composition of claim 29 wherein the quinolone comprises a fluoroquinolone.

32. The composition of claim 31 wherein the fluoroquinolone comprises ciprofloxacin, sitafloxacin, dalofloxacin, antofloxacin, levonadifloxacin, gemifloxacin, acorafloxacin, amifloxacin, avarofloxacin, balofloxacin, benofloxacin, besifloxacin, cadroflocacin, clinafloxacin, danofloxacin, ecenofloxacin, enoxacin, enrofloxacin, esafloxacin, finafloxacin, fleroxacin, gatifloxacin, grepafloxacin, irloxacin, lemefloxacin, levofloxacin, lomefloxacin, marbofloxacin, merafloxacin, motifloxacin, nadifloxacin, orbifloxacin, pazufloxacin, pefloxacin, pradofloxacin, premafloxacin, rosoxacin, rufloxacin, sarafloxacin, temafloxacin, trovafloxacin, ulifloxacin, vebufloxacin.

33. The composition of claim 29 wherein the antibiotic comprises a beta-lactam.

34. The composition of claim 33 wherein the beta-lactam comprises a carbapenem.

35. The composition of claim 30 wherein the first moiety comprises ascorbic acid or an ascorbic acid derivative.

36. The composition of claim 30 wherein the antibacterial agent comprises a beta-lactam.

37. The composition of claim 36 wherein the beta-lactam comprises a carbapenem.

38. The composition of claim 37 wherein the carbapenem comprises imipenem, meropenem, panipenem, biapenem, ertapenem, or tebipenem.

39. The composition of claim 36 wherein the first moiety comprises ascorbic acid or an ascorbic acid derivative.

40. The composition of claim 27 wherein the antimicrobial agent comprises an antiviral agent.

41. The composition of claim 40 wherein the antiviral agent comprises an adamantane antiviral, e.g., amantadine, rimantadine; an antiviral interferon, e.g., peginterferon alfa-2b, peginterferon alfa-2s, peginterferon alfa-2b; a chemokine receptor antagonist, e.g. maraviroc; an integrase strand transfer inhibitor, e.g. raltegravir, dolutegravir, elvitegravir; a neuraminidase inhibitor, e.g., zanamivir, oseltamivir, peramivir; a non-nucleoside reverse transcriptase inhibitor (NNRTI), e.g., etravirine, efavirenz, nevirapine, rilpivirine, doravirine, delavirdine; a non-structural protein 5A (Ns5A) inhibitor, e.g., daclatasivir; a nucleoside reverse transcriptase inhibitor (NRTI), e.g., kentecavir, lamivudine, adefovir, didanosine, tenofovir alafenamide, tenofovir, zidovudine, stavudine, emtricitabine, zalcitabine, telbivudine; a protease inhibitor, e.g., boceprevir, simeprevir, fosamprenavir, lopinavir, ritonavir, darunavir, telaprevir, tipranavir, atazanavir, nelfinavir, amprenavir, indinavir, saquinavir; a purine nucleoside, e.g., ribavirin, valacyclovir, acyclovir, famiciclovir, valganciclovir, ganciclovir, cidofovir. An antiviral booster is used in certain embodiments, e.g., ritonavir, cobicistat

42. The composition of claim 27 wherein the antimicrobial agent comprises an antifungal agent.

43. The composition of claim 42 wherein the antifungal agent comprises amphotericin B; an azole derivative, e.g., ketoconazole, fluconazole, itraconazole, posaconazole, voriconazole; an echinocandin, e.g., anidulafungin, caspofungin, micafungin; flucytosine.

44. The composition of claim 27 wherein the antimicrobial agent comprises an antiparasitic agent.

45. The composition of claim 44 wherein the antiparasitic agent comprises an antimalarial agent.

46. The composition of claim 1 wherein the first and second moieties are linked covalently.

47. The composition of claim 46 wherein the covalent linkage comprises an ester, carbonate, amide, imine, acetal or ether linkage or a combination thereof.

48. The composition of claim 46 wherein the covalent linkage between the first and second moieties is a direct covalent linkage.

49. The composition of claim 46 wherein the covalent linkage between the first and second moieties is via a linkage moiety.

50. The composition of claim 46 wherein the covalent linkage is configured to be broken after the composition interacts with the cell that participates in infection healing.

51. The composition of claim 50 wherein the covalent linkage is configured to be broken in the presence of reactive oxygen species (ROS), in a low pH environment, or both.

52. The composition of claim 50 wherein the covalent linkage is hydrolytically stable.

53. The composition of claim 51 wherein the linkage comprises acetal-boronate.

54. The composition of claim 1 wherein the first and second moieties are linked noncovalently.

55. The composition of claim 1 wherein the first moiety comprises a first antimicrobial agent and the second moiety comprises a second antimicrobial agent, wherein the first and second antimicrobial agent are different.

56. The composition of claim 55 wherein the first antimicrobial agent comprises a fluoroquinolone, a tetracycline, or a macrolide.

57. The composition of claim 1 wherein the first moiety and the second moiety comprise areas of an antimicrobial agent.

58. A composition comprising an infection healing cell comprising an antimicrobial agent.

59. The infection healing cell of claim 58 wherein the antimicrobial agent comprises an antibacterial, an antiviral, an antifungal, or an antiparasitic agent.

60. The infection healing cell of claim 58 wherein the antimicrobial is present at a concentration of at least 1 ng/ml.

61. The infection healing cell of claim 58 wherein the infection healing cell is in an aqueous environment, and wherein the antimicrobial agent is present in the intracellular environment of the infection healing cell at a first concentration and in the extracellular aqueous environment at a second concentration, and wherein the ratio of first to second concentration is at least 2, 3, 4, 5, 6, 7, 8, 9, 10, 12, 15, 17, 20, 22, 25, 27, 30, 35, 40, 50, 60, 70, 80, 100.

62. The infection healing cell of claim 58 wherein the antimicrobial agent comprises an antibacterial agent.

63. The infection healing cell of claim 62 wherein the antibacterial agent comprises a fluoroquinolone or a beta-lactam.

64. The infection healing cell of claim 63 wherein the antimicrobial comprises a beta-lactam, a cephalosporin.

65. The infection healing cell of claim 58 wherein the antimicrobial is associated with the surface of the immune cell.

66. The infection healing cell of claim 58 wherein the antimicrobial is intracellular.

67. The infection healing cell of claim 66 wherein at least 50% of the antimicrobial is located in the cytosol.

68. The infection healing cell of claim 68 wherein the infection healing cell is capable of normal or substantially normal function.

69. The immune cell of claim 58 wherein the antimicrobial is linked to a moiety that interacts with a moiety of the infection healing cell.

70. A composition for treating a site of a drug-resistant bacterial infection comprising
(i) an antibiotic specific for the drug-resistant bacteria linked to
(ii) a ligand that targets infection healing cells at the site of infection or drawn to the site of infection.

71. A composition comprising
(i) a first antimicrobial agent that is preferentially accumulated by one or more types of infection healing cells, linked to
(ii) a second antimicrobial agent.

72. The composition of claim 71 where in the first and second antimicrobial agents are different.

73. The composition of claim 71 wherein the first and second antimicrobial agents are the same type of antimicrobial agent.

74. The composition of claim 71 wherein the infection healing cell comprises an immune cell.

75. The composition of claim 74 wherein the immune cell comprises a phagocyte.

76. The composition of claim 71 wherein the infection healing cell comprises a wound repair cell.

77. The composition of claim 76 wherein the wound repair cell comprises a fibroblast.

78. The composition of claim 71 wherein the first antimicrobial agent comprises a macrolide.

79. The composition of claim 71 wherein the first antimicrobial agent comprises a fluoroquinolone.

80. The composition of claim 78 wherein the macrolide comprises azithromycin.

81. The composition of claim 71 wherein the second antimicrobial agent comprises a fluoroquinolone.

82. The composition of claim 79 wherein the second antimicrobial agent comprises a beta-lactam.

83. A method of accumulating an antimicrobial agent in a cell comprising
(i) contacting the cell extracellularly with the antimicrobial agent linked to a ligand that interacts with a cell that participates in infection healing to concentrate the first ligand on or in the cell; (ii) allowing the antimicrobial agent linked to the ligand to accumulate in the cell.

84. The method of claim 83 further comprising
(iii) cleaving the linkage between the ligand and the antimicrobial agent to release the agent in active form.

85. A method of delivering an antimicrobial agent to a site of an infection, mediated by one or more microbial agents, in an individual comprising
(i) administering to the individual a composition comprising an antimicrobial agent linked to a ligand that interacts with an infection healing cell to concentrate the antimicrobial agent at the infection healing cell, wherein the infection healing cell is a cell that is present at the site of infection or that preferentially travels to the site of infection; and
(ii) causing the antimicrobial agent to interact with the one or more microbial agents at the site of infection.

86. The method of claim 85 wherein step (iii) comprises lysis of the infection healing cell.

87. The method of claim 85 wherein at least one of the one or more microbial agents comprises an antibiotic-resistant bacterium.

88. A composition comprising
(i) a ligand that interacts with a moiety associated with an infection healing cell;
(ii) a linker covalently linked to the ligand; and
(iii) an antibiotic covalently linked to the ligand.

89. A method of treating an infection caused by one or more microbial agents in an individual suffering from the infection comprising administering to the individual an effective amount of a composition comprising an antimicrobial agent effective against the one or more microbial agents linked to a ligand that interacts with an infection healing cell to concentrate the antimicrobial agent at the infection healing cell.

90. A pharmaceutical composition comprising a composition comprising an antimicrobial agent effective against one or more microbial agents linked to a ligand that interacts with a cell that participates in infection healing to concentrate the antimicrobial agent at the cell, and a pharmaceutically acceptable excipient.

91. A composition comprising
(i) ascorbic acid or an ascorbic acid derivative, linked to
(ii) an antimicrobial agent.

92. The composition of claim 91 wherein the ascorbic acid or ascorbic acid derivative and the antimicrobial agent are linked noncovalently.

93. The composition of claim 91 wherein the ascorbic acid or ascorbic acid derivative and the antimicrobial agent are linked covalently.

94. The composition of claim 91 wherein the antimicrobial agent comprises an antibiotic.

95. The composition of claim 94 wherein the antibiotic is a fluoroquinolone or a beta-lactam.

96. The composition of claim 94 wherein the antibiotic comprises carbapenem.

97. The composition of claim 91 wherein the linker is hydrolytically stable but cleaved by reactive oxygen species (ROS).

98. The composition of claim 97 wherein the linker comprises acetal-boronate.

99. A composition comprising
(i) a first antimicrobial agent that interacts with an infection healing cell in such a way as to increase the concentration of the antimicrobial agent at the infection healing cell cell, linked to
(ii) a second antimicrobial agent.

100. The composition of claim 99 wherein the first and second antimicrobial agents are two of the same agent.

101. The composition of claim 99 wherein the first and second antimicrobial agents are different.

102. A composition comprising
(i) a ligand targeting a target moiety associated with a natural killer (NK) or a T cell linked to
(ii) a moiety comprising an antiviral agent.

103. A composition comprising
(i) a ligand targeting a target moiety associated with a monocyte/macrophage linked to
(ii) a moiety comprising an antifungal agent.

104. A composition comprising
(i) a first moiety linked to
(ii) a second moiety; wherein the first and second moieties are linked via a linker comprising acetal-boronate.

105. A method of transporting an antimicrobial agent into a cell comprising contacting the cell with an effective amount of a composition comprising a ligand for a transporter in the plasma membrane of the cell linked to the antimicrobial agent under conditions wherein the ligand binds to the transporter and is carried into the cell along with the antimicrobial agent.

106. A composition comprising
(i) an infection healing cell comprising a membrane transporter for transporting a ligand across a cell membrane of the infection healing cell;
(ii) a ligand or a derivative of the ligand, linked to an antimicrobial agent, wherein the ligand or ligand derivative is attached to the transporter, or is inside the infection healing cell.
